(19) United States
(12) Patent Application Publication
(10) Pub. No.: US 2025/0125050 A1
(43) Pub. Date: Apr. 17, 2025

(54) SYSTEMS AND METHODS FOR MOLECULAR RESIDUAL DISEASE LIQUID BIOPSY ASSAY

(71) Applicant: Tempus AI, Inc., Chicago, IL (US)

(72) Inventors: Ana Sofia de Olazarra, Chicago, IL (US); Terri M. Driessen, Chicago, IL (US); Jon Foox, New York, NY (US); Ailin Jin, San Jose, CA (US); Christine Y. Lee, Menlo Park, CA (US); Daniel Sherman Neems, Evanston, IL (US); Anne Franklin Sonnenschein, Chicago, IL (US); Robert Tell, Chicago, IL (US); Jason Yamada-Hanff, Los Angeles, CA (US)

(21) Appl. No.: 18/913,883

(22) Filed: Oct. 11, 2024

Related U.S. Application Data
(60) Provisional application No. 63/590,386, filed on Oct. 13, 2023.
(60) Provisional application No. 63/654,665, filed on May 31, 2024.

(51) Int. Cl. G16H 50/20; G16B 20/10; G16B 20/20; G16H 20/10

(52) U.S. Cl. CPC G16H 50/20; G16B 20/10; G16B 20/20; G16H 20/10

(57) ABSTRACT

Systems and methods for molecular residual disease (MRD) status determination for a subject's cancer condition subject uses sequence reads from methylation sequencing of cell-free DNA fragments from the subject's liquid biopsy sample to determine their methylation patterns. From this, corresponding numbers of circulating-tumor DNA (ctDNA) fragments mapping to each of a plurality of regions of the genome of the subject's species are determined. Corresponding expected numbers of noise fragments in each region are determined based on corresponding distributions using observed sequencing depths and learned background emission rates for each region. An excess fragments per million (FPM) value, corrected by an observed CHG methylation level, is determined from the observed number of ctDNA fragments in excess of the expected number of noise fragments in each region. MRD is called when the CHG corrected excess FPM value satisfies a threshold value, and a call against MRD is made when it does not.

### CROSS-REFERENCE TO RELATED PATENT APPLICATIONS

This application claims priority to U.S. Provisional Patent Application No. 63/590,386, entitled “Systems and Methods for Molecular Residual Disease Liquid Biopsy Assay,” filed Oct. 13, 2023, which is hereby incorporated by reference. This application also claims priority to U.S. Provisional Patent Application No. 63/654,665, entitled “Systems and Methods for Molecular Residual Disease Liquid Biopsy Assay,” filed May 31, 2024, which is hereby incorporated by reference.

### TECHNICAL FIELD

Disclosed are technologies generally relating to screening for molecular residual disease in subjects treated for cancer conditions using liquid biopsy samples.

### BACKGROUND

MRD, or molecular residual disease, represents a small number of cancer cells left in the body after treatment that are not detectable by imaging or traditional blood tests. These cells have the potential to come back and cause cancer relapse in cancer patients. MRD is detected by assaying for circulating tumor DNA (ctDNA) in treated patients.

A major challenge for detecting ctDNA MRD after cancer treatment remains the low levels of ctDNA in plasma, often at levels below 0.1% (as a percentage of total cfDNA). For example, see Chin et al., 2019, “Detection of solid tumor molecular residual disease (MRD) using circulating tumor DNA (ctDNA),” Mol Diagn Ther 23, pp. 311-331. Additionally, clonal hematopoiesis of indeterminate potential (CHIP) is present at low levels in plasma and can be a confounder of ctDNA detection specificity. For example, see Pellini et al., 2020, “Liquid biopsies using circulating tumor DNA in non-small cell lung cancer,” Thorac Surg Clin 30:165-177, Chin, Id., Swanton et al., 2018, “Prevalence of clonal hematopoiesis of indeterminate potential (CHIP) measured by an ultra-sensitive sequencing assay: Exploratory analysis of the Circulating Cancer Genome Atlas (CCGA) study,” J Clin Oncol 36, 2018. (suppl 15; abstr 12003); and Hu et al., 2018, “False-positive plasma genotyping due to clonal hematopoiesis, Clin Cancer Res 24:4437-4443. Background error rates can further compromise the performance of NGS-based ctDNA assays. See Kurtz et al., 2021, “Enhanced detection of minimal residual disease by targeted sequencing of phased variants in circulating tumor DNA,” Nat Biotechnol 39:1537-1547.

Given the above background, systems and methods for improved molecular residual disease detection are needed in the art. Such assays need to have exceptional sensitivity and specificity to detect tumoral genomic alterations that are often present at ultralow levels in plasma. Such assays also need to not falsely call CHIP mutations and not adversely be affected by background error rates.

### SUMMARY

The present disclosure addresses the above-identified shortcomings by providing systems and methods for determining whether a subject has a positive or negative molecular residual disease status for a cancer condition.

In some embodiments, the cancer condition is a particular type of cancer.

In some embodiments, the cancer condition is a particular stage of a particular type of cancer.

A corresponding nucleic acid sequence of each cell-free DNA fragment in a first plurality of cell-free DNA fragments is obtained from a first plurality of sequence reads of a first sequencing reaction. The first sequencing reaction is a methylation sequencing of the first plurality of cell-free DNA fragments from a first liquid biopsy sample of the test subject. Each respective nucleic acid sequence in the first plurality of nucleic acid sequences comprises a methylation pattern for a corresponding cell-free DNA fragment in the first plurality of cell-free DNA fragments.

In some embodiments, the first liquid biopsy sample is blood. In some embodiments, the first liquid biopsy sample comprises blood, whole blood, peripheral blood, plasma, serum, or lymph of the test subject. In some embodiments, the volume of the first liquid biopsy sample is less than 30 mL

In some embodiments, the first sequencing reaction is a whole genome methylation sequencing. In some embodiments, the first sequencing reaction is a whole genome sequencing.

In some embodiments, the first plurality of sequence reads comprises at least 50,000 sequence reads or at least 250,000 sequence reads.

A corresponding number of circulating-tumor fragments mapping to each respective region in a plurality of regions of one or more first reference sequences of the species of the test subject is determined using a methylation pattern of each nucleic acid sequence in the first plurality of nucleic acid sequences.

Further, a corresponding expected number of noise fragments in each respective region of the plurality of regions is determined based on a corresponding distribution for the respective region using an observed sequencing depth from the first sequence reaction and a learned background emission rate for the respective region.

An excess fragments per million value for the first liquid biopsy sample is determined from the corresponding number of ctDNA fragments in each respective region of the plurality of regions in excess of each corresponding expected number of noise fragments in each respective region.

In some embodiments, the excess fragments per million value is corrected for the first liquid biopsy sample by an observed CHG methylation level to obtain a corrected excess fragments per million value.

A first threshold is applied to the corrected excess fragments per million value to provide a first call for molecular residual disease when the corrected excess fragments per million value satisfies the first threshold or a first call against molecular residual disease when the corrected excess fragments per million value fails to satisfy the first threshold.

In some embodiments, additional information is used to check for molecular residual disease. In such embodiments, a second sequencing reaction is performed in which a corresponding sequence of each cell-free DNA fragment in a second plurality of cell-free DNA fragments in a second liquid biopsy sample of the test subject is sequenced, thereby obtaining a second plurality of sequence reads.

In some embodiments, the first liquid biopsy sample and the second liquid biopsy sample are the same liquid biopsy sample. In some embodiments, the first liquid biopsy sample and the second liquid biopsy sample are different liquid biopsy samples.

In some embodiments, the second sequencing reaction is a panel-based sequencing reaction of a plurality of loci. In some such embodiments, the plurality of loci is sequenced at an average sequence depth of at least 250× by the second sequencing reaction. In some such embodiments, the plurality of loci is sequenced at an average sequence depth of at least 1000× by the second sequencing reaction.

In some embodiments, the second plurality of sequence reads comprises at least 50,000 sequence reads or at least 250,000 sequence reads.

In some embodiments, the one or more first reference sequences is a human reference genome, the one or more second reference sequences is also the human reference genome, the plurality of regions comprises 1000 or more regions cumulatively mapping to between four megabases and ten megabases of the human reference genome, the second sequencing reaction is a panel-based sequencing reaction of a plurality of loci, and the plurality of loci comprises 50 or more loci cumulatively mapping to between 0.1 megabase and 1 megabase of the human reference genome.

In some embodiments, the second plurality of sequence reads is used to identify a respective variant allele fraction for each respective variant in a set of candidate somatic variants. Each candidate somatic variant in the set of candidate somatic variants is a single nucleic acid variant (SNV).

In some such embodiments, each respective candidate somatic variant in the set of candidate somatic variants is identified by applying a variant caller to the second plurality of sequence reads with a restriction that the variant caller determines that each respective candidate somatic variant in the set of candidate somatic variants has a variant allele frequency of at least 0.1 in the second plurality of sequence reads and that at least one cell-free DNA fragment in the second plurality of cell-free DNA fragments exhibits the respective candidate somatic variant.

In some embodiments, the set of candidate somatic variants is filtered by a procedure.

In some embodiments, the procedure comprises removing from the set of candidate somatic variants each respective candidate somatic variant in the set of candidate somatic variants that is present in a repository of known germline variants.

In some embodiments, the procedure comprises removing from the set of candidate somatic variants each respective candidate somatic variant in the set of candidate somatic variants that maps to a respective variant interval identified as having pre-test odds of a positive variant call that is less than a pre-test odds threshold value based upon a prevalence of a corresponding one or more training variants, which are each above a limit of detection and map to the respective variant interval, in a plurality of tumor samples obtained from a first cohort of tumor-normal matched training subjects having the cancer condition with the proviso that no variant in a second cohort of healthy samples maps to the respective variant interval.

In some such embodiments, the procedure comprises removing from the set of candidate somatic variants each respective candidate somatic variant in the set of candidate somatic variants that is identified as an artifactual variant or that is identified as being observed in a cohort of healthy subjects.

In some embodiments, the procedure comprises removing from the set of candidate somatic variants each respective candidate somatic variant in the set of candidate somatic variants in which the second sequencing reaction produced a coverage depth of less than a threshold amount for the respective locus in one or more second reference sequences of the species of the subject that the candidate somatic variant maps to.

In some embodiments, the procedure comprises removing from the set of candidate somatic variants each respective candidate somatic variant in the set of candidate somatic variants that fails to be represented by at least one cell-free DNA fragment in the second plurality of cell-free DNA fragments in which both strands of the at least one cell-free DNA fragment are identified in one or more sequence reads of the second plurality of sequence reads.

In some embodiments, the procedure comprises removing from the set of candidate variants each respective candidate somatic variant in the set of candidate somatic variants that (a) maps to a repeat region in the one or more second reference sequences of the species and (b) is not annotated as a known somatic mutation in a database of known somatic mutations for the species of the subject.

In some embodiments, the procedure comprises removing from the set of candidate somatic variants each respective candidate somatic variant in the set of candidate somatic variants that maps to a region of clonal hematopoiesis of indeterminate potential (CHIP). In some embodiments the region of CHIP is SXL1, BCOR, BCORL1, CBL, CREBBP, CUX1, DNMT3A, GNB1, JAK2, PPMID, PRPF8, SETDB1, SF3B1, SRSF2, TET2, U2AF1, or any combination thereof. In some embodiments the region of CHIP is TET2, DNMT3A, ASXL1, SF3B1, or any combination thereof. In some embodiments, the region of CHIP is TET2, DNMT3A, ASXL1, SF3B1, CBL, U2AF1, IDH2,2,3, MYD88,13, EP300, CDKN2C, HNF1A, or any combination thereof.

In some embodiments, a second call for molecular residual disease is provided when there remains a candidate variant in the set of candidate variants after application of the procedure, or a second call against molecular residual disease is provided when no candidate variant remains in the set of candidate variants after application of the procedure.

In some embodiments, (i) an indication that the subject has positive molecular residual disease status for the cancer condition is provided when the first call for molecular residual disease has been made or (ii) an indication that the subject has negative molecular residual disease status for the cancer condition when the first call against molecular residual disease has been made.

In some embodiments, the indication that the subject has positive molecular residual disease status for the cancer condition is provided when the first call for molecular residual disease is made or the second call for molecular residual disease is made.

In some embodiments, the indication that the subject has negative molecular residual disease status for the cancer condition is provided when the first call against molecular residual disease and the second call against molecular residual disease is made.

In some embodiments, a report is generated for the subject comprising the identity of candidate variants remaining in the set of candidate variants after running the procedure. In some embodiments, the report further comprises a therapeutic match for the subject based on an identity of one or more of the candidate variants remaining in the set of candidate variants after running the procedure.

Another aspect of the present disclosure provides a system comprising a processor and a memory storing instructions, which when executed by the processor, cause the processor to perform steps comprising any of the methods described in the present disclosure.

Still another aspect of the present disclosure provides a non-transitory computer-readable medium storing computer code comprising instructions, when executed by one or more processors, causing the processors to perform any of the methods described in the present disclosure.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 illustrates an exemplary system for determining whether a subject has a positive or negative molecular residual disease status for a cancer condition, in accordance with some embodiments of the present disclosure.

FIGS. 2A, 2B, 2C, 2D, 2E, 2F, and 2G provide an example flowchart depicting an example process for determining whether a subject has a positive or negative molecular residual disease status for a cancer condition, in which optional elements are denoted by dashed boxes, in accordance with some embodiments of the present disclosure.

FIG. 3 illustrates an example workflow for determining whether a subject has a positive or negative molecular residual disease status for a cancer condition, in accordance with some embodiments of the present disclosure.

FIGS. 4A, 4B, 4C, 4D, 4E, 4F, 4G, and 4H provide another example flowchart depicting an example process for determining whether a subject has a positive or negative molecular residual disease status for a cancer condition, in which optional elements are denoted by dashed boxes, in accordance with some embodiments of the present disclosure.

FIG. 5 illustrates example model performance for a cohort of 70 early stage colorectal cancer subjects where the data for the model is prepared in accordance with FIG. 2, in accordance with an embodiment of the present disclosure.

FIG. 6 illustrates the model performance of FIG. 5 broken down by site of cancer recurrence, in accordance with an embodiment of the present disclosure.

FIG. 7 illustrates disease free survival (DFS) by landmark 1-month post-surgery MRD status for the subjects summarized in FIG. 5 with greater than 1 year follow-up, in accordance with an embodiment of the present disclosure.

FIG. 8 illustrates example model performance for a cohort of early stage colorectal cancer subjects where the data for the model is prepared in accordance with FIG. 4, in accordance with an embodiment of the present disclosure.

FIG. 9 illustrates distribution of lead time (time from first MRD+ call to date of recurrence or death) for true positive subjects of the study summarized in FIG. 8, in accordance with an embodiment of the present disclosure.

FIG. 10 illustrates clinical landmark performance (top) and clinical longitudinal performance (bottom) of the study summarized in FIG. 8, in accordance with an embodiment of the present disclosure.

FIGS. 11A and 11B illustrate adjusted hazard ratio (HR) was nearly 5-fold higher compared to carcinoembryonic antigen (CEA) testing at 12 weeks post-surgery, where adjusted HR* was the hazard ratio adjusted by anticipated true recurrence rate (24%), and that the adjusted median disease free survival (DFS) time for MRD+ is 25.1 weeks (6.3 months) versus not reached within 72 weeks (18 months) for MRD-, for the study summarized in FIG. 8, in accordance with an embodiment of the present disclosure.

FIG. 12 illustrates probes that include all four potential sequences at a given loci: methylated, unmethylated, sense and antisense in accordance with the prior art as disclosed on the Internet at twistbioscience.com/blog/science/tools-improve-methyl-seq-efficiency-better-resolution-epigenetic-studies, last accessed Oct. 11, 2024

FIG. 13 illustrates different ratios of an APOBEC mix (enzyme, buffer, and bovine serum albumin (BSA)) used in a sequencing reaction in accordance with some embodiments of the present disclosure.

### DETAILED DESCRIPTION

The present disclosure addresses the challenges in the art described in the above background by providing systems and methods for determining molecular residual disease (MRD) status for a subject's cancer condition subject using sequence reads from methylation sequencing of cell-free DNA fragments from the subject's liquid biopsy sample to determine their methylation patterns. Such patterns are used to map a corresponding number of circulating-tumor DNA (ctDNA) fragments to each region in a plurality of regions of one or more reference sequences (e.g., a genome) of the subject's species. Corresponding expected numbers of noise fragments in each region are determined based on corresponding distributions using observed sequencing depths and learned background emission rates for each region. An excess fragments per million (FPM) value, corrected by an observed CHG methylation level, is determined from the observed number of ctDNA fragments in excess of the expected number of noise fragments in each region. A call for MRD is made when the CHG corrected excess FPM value satisfies a threshold value and a call against MRD is made when it does not. In some embodiments this call for MRD is supplemented by an additional cell-free DNA fragment workflow that looks for alterations in high confidence variant intervals.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

Plural instances may be provided for components, operations or structures described herein as a single instance. Finally, boundaries between various components, operations, and data stores are somewhat arbitrary, and particular operations are illustrated in the context of specific illustrative configurations. Other forms of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms “first,” “second,” etc. may be used herein to describe various elements, these elements should not be limited by these terms. These terms are only used to distinguish one element from another. For example, a first dataset could be termed a second dataset, and, similarly, a second dataset could be termed a first dataset, without departing from the scope of the present invention. The first dataset and the second dataset are both datasets, but they are not the same dataset.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined (that a stated condition precedent is true)” or “if (a stated condition precedent is true)” or “when (a stated condition precedent is true)” may be construed to mean “upon determining” or “in response to determining” or “in accordance with a determination” or “upon detecting” or “in response to detecting” that the stated condition precedent is true, depending on the context.

Furthermore, when a reference number is given an “i^{th}” denotation, the reference number refers to a generic component, set, or embodiment. For instance, a cellular-component termed “cellular-component i” refers to the i^{th} cellular-component in a plurality of cellular-components.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention.

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

### Definitions

Unless defined otherwise, all technical and scientific terms used herein have the meaning commonly understood by one of ordinary skill in the art to which the invention pertains.

As used herein, the terms “abundance,” “abundance level,” or “expression level” refers to an amount of a cellular constituent (e.g., a gene product such as an RNA species, e.g., mRNA or miRNA, or a protein molecule) present in one or more cells, or an average amount of a cellular constituent present across multiple cells. When referring to mRNA or protein expression, the term generally refers to the amount of any RNA or protein species corresponding to a particular genomic locus, e.g., a particular gene. However, in some embodiments, an abundance can refer to the amount of a particular isoform of an mRNA or protein corresponding to a particular gene that gives rise to multiple mRNA or protein isoforms. The genomic locus can be identified using a gene name, a chromosomal location, or any other genetic mapping metric.

As used herein, the term “allele” refers to a particular sequence of one or more nucleotides at a chromosomal locus. In a haploid organism, the subject has one allele at every chromosomal locus. In a diploid organism, the subject has two alleles at every chromosomal locus.

As used herein the term “cancer,” “cancerous tissue,” or “tumor” refers to an abnormal mass of tissue in which the growth of the mass surpasses, and is not coordinated with, the growth of normal tissue, including both solid masses (e.g., as in a solid tumor) or fluid masses (e.g., as in a hematological cancer). A cancer or tumor can be defined as “benign” or “malignant” depending on the following characteristics: degree of cellular differentiation including morphology and functionality, rate of growth, local invasion and metastasis. A “benign” tumor can be well differentiated, have characteristically slower growth than a malignant tumor and remain localized to the site of origin. In addition, in some cases a benign tumor does not have the capacity to infiltrate, invade or metastasize to distant sites. A “malignant” tumor can be poorly differentiated (anaplasia), have characteristically rapid growth accompanied by progressive infiltration, invasion, and destruction of the surrounding tissue. Furthermore, a malignant tumor can have the capacity to metastasize to distant sites. Accordingly, a cancer cell is a cell found within the abnormal mass of tissue whose growth is not coordinated with the growth of normal tissue. Thus, a “tumor sample” refers to a biological sample obtained or derived from a tumor of a subject, as described herein.

As used herein, the term “cell-free DNA” and “cfDNA” interchangeably refer to DNA fragments that circulate in a subject's body (e.g., bloodstream) and originate from one or more healthy cells and/or from one or more cancer cells. These DNA molecules are found outside cells, in bodily fluids such as blood, whole blood, plasma, serum, urine, cerebrospinal fluid, fecal, saliva, sweat, sweat, tears, pleural fluid, pericardial fluid, or peritoneal fluid of a subject, and are believed to be fragments of genomic DNA expelled from healthy and/or cancerous cells, e.g., upon apoptosis and lysis of the cellular envelope.

As used herein, the terms “genomic alteration,” “mutation,” and “variant” interchangeably refer to a detectable change in the genetic material of one or more cells. A genomic alteration, mutation, or variant can refer to various type of changes in the genetic material of a cell, including changes in the primary genome sequence at single or multiple nucleotide positions, e.g., a single nucleotide variant (SNV), a multi-nucleotide variant (MNV), an indel (e.g., an insertion or deletion of nucleotides), a DNA rearrangement (e.g., an inversion or translocation of a portion of a chromosome or chromosomes), a variation in the copy number of a locus (e.g., an exon, gene, or a large span of a chromosome) (CNV), a partial or complete change in the ploidy of the cell, as well as in changes in the epigenetic information of a genome, such as altered DNA methylation patterns. In some embodiments, a mutation is a change in the genetic information of the cell relative to a particular reference genome, or one or more ‘normal’ alleles found in the population of the species of the subject. For instance, mutations can be found in both germline cells (e.g., non-cancerous, ‘normal’ cells) of a subject and in abnormal cells (e.g., pre-cancerous or cancerous cells) of the subject. As such, a mutation in a germline of the subject (e.g., which is found in substantially all ‘normal cells’ in the subject) is identified relative to a reference genome for the species of the subject. However, many loci of a reference genome of a species are associated with several variant alleles that are significantly represented in the population of the subject and are not associated with a diseased state, e.g., such that they would not be considered ‘mutations.’ By contrast, in some embodiments, a mutation in a cancerous cell of a subject can be identified relative to either a reference genome of the subject or to the subject's own germline genome. In certain instances, identification of both types of variants can be informative. For instance, in some instances, a mutation that is present in both the cancer genome of the subject and the germline of the subject is informative for precision oncology when the mutation is a so-called ‘driver mutation,’ which contributes to the initiation and/or development of a cancer. However, in other instances, a mutation that is present in both the cancer genome of the subject and the germline of the subject is not informative for precision oncology, e.g., when the mutation is a so-called ‘passenger mutation,’ which does not contribute to the initiation and/or development of the cancer. Likewise, in some instances, a mutation that is present in the cancer genome of the subject but not the germline of the subject is informative for precision oncology, e.g., where the mutation is a driver mutation and/or the mutation facilitates a therapeutic approach, e.g., by differentiating cancer cells from normal cells in a therapeutically actionable way. However, in some instances, a mutation that is present in the cancer genome but not the germline of a subject is not informative for precision oncology, e.g., where the mutation is a passenger mutation and/or where the mutation fails to differentiate the cancer cell from a germline cell in a therapeutically actionable way.

As used herein, the term “germline variants” refers to genetic variants inherited from maternal and paternal DNA. In some embodiments, germline variants are determined through a matched tumor-normal calling pipeline.

As used herein, the term “mapping” refers to assigning a sequence read to a larger sequence, e.g., a reference sequence such as a genome. In some embodiments, mapping is performed by alignment. For instance, the mapping of a sequence read to a reference genome determines the locus in the reference genome that best matches the sequence of the sequence read.

As used herein, the term “measure of central tendency” refers to a central or representative value for a distribution of values. Non-limiting examples of measures of central tendency include an arithmetic mean, weighted mean, midrange, midhinge, trimean, geometric mean, geometric median, Winsorized mean, median, and mode of the distribution of values.

As used herein, “messenger RNA” or “mRNA” are RNA molecules comprising a sequence that encodes a polypeptide or protein. In general, RNA can be transcribed from DNA. In some cases, precursor mRNA containing non-protein coding regions in the sequence can be transcribed from DNA and then processed to remove all or a portion of the non-coding regions (introns) to produce mature mRNA. As used herein, the term “pre-mRNA” can refer to the RNA molecule transcribed from DNA before undergoing processing to remove the non-protein coding regions.

As used herein, unless otherwise dictated by context “nucleotide” or “nt” refers to ribonucleotide.

As used herein, the terms “patient” and “subject” are used interchangeably, and may be taken to mean any living or non-living organism including, but not limited to, a human (e.g., a male human, female human, fetus, pregnant female, child, or the like), a non-human mammal, or a non-human animal. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any age (e.g., a man, a woman, or a child).

As used herein, the term “reference allele” refers to the sequence of one or more nucleotides at a chromosomal locus that is either the predominant allele represented at that chromosomal locus within the population of the species (e.g., the “wild-type” sequence), or an allele that is predefined within a reference genome for the species.

As disclosed herein, the term “reference genome” or “genome” refers to any known, sequenced, or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

As used herein, the term “sensitivity,” “recall,” or “true positive rate” (TPR) refers to the number of true positives divided by the sum of the number of true positives and false negatives. Sensitivity can characterize the ability of an assay or method to correctly identify a proportion of the population that truly has a condition. For example, sensitivity can characterize the ability of a method to correctly identify the number of subjects within a population having cancer. In another example, sensitivity can characterize the ability of a method to correctly identify the one or more markers indicative of cancer.

The terms “sequence reads” or “reads,” used interchangeably herein, refer to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130 bp, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp or more. Nanopore sequencing, for example, can provide sequence reads that vary in size from tens to hundreds to thousands of base pairs. Illumina parallel sequencing can provide sequence reads that vary to a lesser extent (e.g., where most sequence reads are of a length of about 200 bp or less). A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes (e.g., in hybridization arrays or capture probes) or amplification techniques, such as the polymerase chain reaction (PCR) or linear amplification using a single primer or isothermal amplification.

As disclosed herein, the terms “sequencing,” “sequence determination,” and the like refer generally to any and all biochemical processes that may be used to determine the order of biological macromolecules such as nucleic acids or proteins. For example, sequencing data can include all or a portion of the nucleotide bases in a nucleic acid molecule such as a DNA fragment.

As used herein, the term “read-depth,” “sequencing depth,” or “depth” can refer to a total number of unique nucleic acid fragments encompassing a particular locus or region of the genome of a subject that are sequenced in a particular sequencing reaction. Sequencing depth can be expressed as “Yx”, e.g., 50×, 100×, etc., where “Y” refers to the number of unique nucleic acid fragments encompassing a particular locus that are sequenced in a sequencing reaction. In such a case, Y is necessarily an integer, because it represents the actual sequencing depth for a particular locus. Alternatively, read-depth, sequencing depth, or depth can refer to a measure of central tendency of the number of unique nucleic acid fragments that encompass one of a plurality of loci or regions of the genome of a subject that are sequenced in a particular sequencing reaction. For example, in some embodiments, sequencing depth refers to the average depth of every locus across an arm of a chromosome, a targeted sequencing panel, an exome, or an entire genome. In such cases, Y may be expressed as a fraction or a decimal, because it refers to an average coverage across a plurality of loci. When a mean depth is recited, the actual depth for any particular locus may be different than the overall recited depth. Metrics can be determined that provide a range of sequencing depths in which a defined percentage of the total number of locus fall. For instance, a range of sequencing depths within which 90% or 95%, or 99% of the loci fall. As understood by the skilled artisan, different sequencing technologies provide different sequencing depths. For instance, low-pass whole genome sequencing can refer to technologies that provide a sequencing depth of less than 5×, less than 4×, less than 3×, or less than 2×, e.g., from about 0.5× to about 3×.

As used herein, the term “duplex sequencing depth” refers to a total number of unique nucleic acid fragments encompassing a particular locus or region of the genome of a subject that are sequenced in a particular sequencing reaction in accordance with the definition for “read-depth” given above. However, for a nucleic acid fragment to be considered sequenced and thus contribute to the duplex sequencing depth, both strands of the nucleic acid fragment need to be sequenced in a duplex sequencing reaction. See Gydush et al., 2022, “Massively-parallel enrichment of minor alleles for mutational testing via low-depth duplex sequencing,” Nat. Biomed Eng, 6(3), pp. 257-266, which is hereby incorporated by reference.

As used herein, the term “somatic variants” refers to variants arising as a result of dysregulated cellular processes associated with neoplastic cells, e.g., a mutation. Somatic variants may be detected via subtraction from a matched normal sample.

As used herein, the term “specificity” or “true negative rate” (TNR) refers to the number of true negatives divided by the sum of the number of true negatives and false positives. Specificity can characterize the ability of an assay or method to correctly identify a proportion of the population that truly does not have a condition. For example, specificity can characterize the ability of a method to correctly identify the number of subjects within a population not having cancer. In another example, specificity characterizes the ability of a method to correctly identify one or more markers indicative of cancer

As used herein, the term “targeted panel” or “targeted gene panel” refers to a combination of probes for sequencing (e.g., by next-generation sequencing) nucleic acids present in a biological sample from a subject (e.g., a tumor sample, liquid biopsy sample, germline tissue sample, white blood cell sample, or tumor or tissue organoid sample), selected to map to one or more loci of interest on one or more chromosomes. In some embodiments, in addition to loci that are informative for precision oncology, a targeted panel includes one or more probes for sequencing one or more of a loci associated with a different medical condition, a loci used for internal control purposes, or a loci from a pathogenic organism (e.g., an oncogenic pathogen).

As used herein, the term “tumor fraction” refers to the fraction of nucleic acid molecules in a sample that originates from a cancerous tissue of the subject, rather than from a noncancerous tissue (e.g., a germline or hematopoietic tissue).

As used herein, the terms “variant” or “mutation” refer to a detectable change in the genetic material of one or more cells. A variant or mutation can refer to various type of changes in the genetic material of a cell, including changes in the primary genome sequence at single or multiple nucleotide positions, e.g., a single nucleotide variant (SNV), a multi-nucleotide variant (MNV), an indel (e.g., an insertion or deletion of nucleotides), a DNA rearrangement (e.g., an inversion or translocation of a portion of a chromosome or chromosomes), a variation in the copy number of a locus (e.g., an exon, gene or a large span of a chromosome) (CNV), a partial or complete change in the ploidy of the cell, and/or changes in the epigenetic information of a genome, such as altered DNA methylation patterns. For example, a single nucleotide variant or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.” In some embodiments, a variant is a change in the genetic information of the cell relative to a particular reference genome or one or more “normal” or “reference” alleles found in the population of the species of the subject. In some embodiments, a variant is a change in the genetic information of the cell relative to a reference cell or tissue, such as a “normal” or “healthy” tissue in the subject. In some embodiments, a variant is a germline mutation or a somatic mutation.

As used herein, the term “variant allele” refers to a sequence of one or more nucleotides at a chromosomal locus that is either not the predominant allele represented at that chromosomal locus within the population of the species (e.g., not the “wild-type” sequence), or not an allele that is predefined within a reference genome for the species.

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are used to implement a methodology in accordance with the features described herein.

### I. EXEMPLARY SYSTEM EMBODIMENTS FOR DETERMINING WHETHER A SUBJECT HAS A POSITIVE OR NEGATIVE MOLECULAR RESIDUAL DISEASE STATUS FOR A CANCER CONDITION

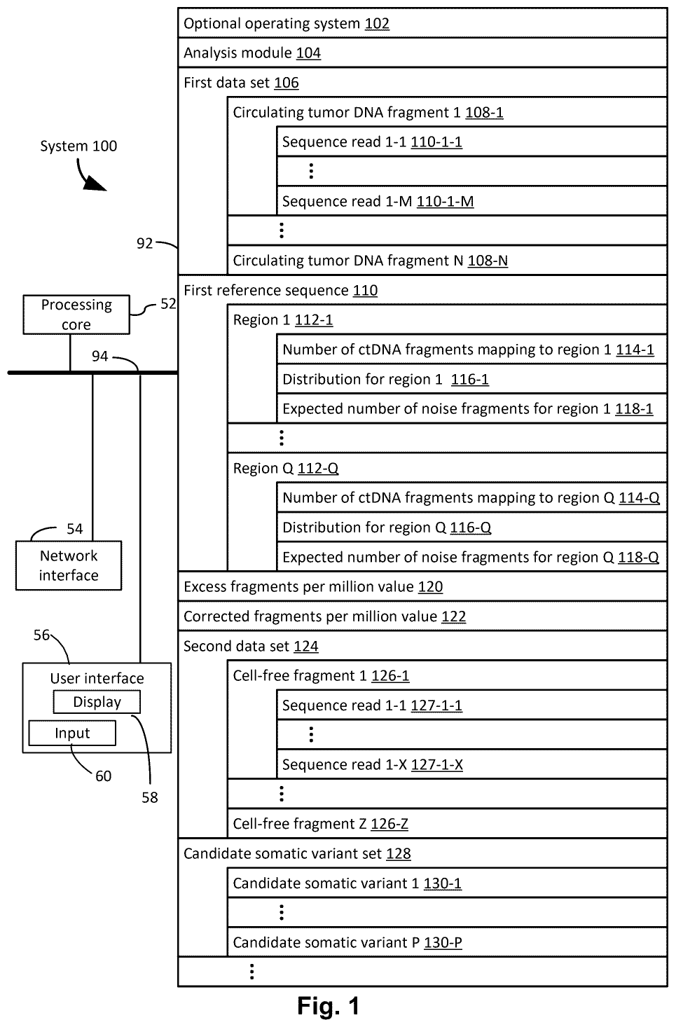

Now that an overview of some aspects of the present disclosure and some definitions used in the present disclosure have been provided, details of an exemplary system are described in conjunction with FIG. 1.

FIG. 1 illustrates a computer system 100 for determining whether a subject has a positive or negative molecular residual disease status for a cancer condition. In typical embodiments, computer system 100 comprises one or more computers. For purposes of illustration in FIG. 1, the computer system 100 is represented as a single computer that includes all of the functionality of the disclosed computer system 100. However, the present disclosure is not so limited. The functionality of the computer system 100 may be spread across any number of networked computers and/or reside on each of several networked computers and/or virtual machines. One of skill in the art will appreciate that a wide array of different computer topologies is possible for the computer system 100 and all such topologies are within the scope of the present disclosure.

Turning to FIG. 1 with the foregoing in mind, the computer system 100 comprises one or more processing units (CPUs) 52, a network or other communications interface 54, a user interface 56 (e.g., including an optional display 58 and optional input 60 (e.g. keyboard or other form of input device)), a memory 92 (e.g., random access memory, persistent memory, or combination thereof), and one or more communication busses 94 for interconnecting the aforementioned components. To the extent that components of memory 92 are not persistent, data in memory 92 can be seamlessly shared with non-volatile memory (not shown) or portions of memory 92 that are non-volatile/persistent using known computing techniques such as caching. Memory 92 can include mass storage that is remotely located with respect to the central processing unit(s) 52. In other words, some data stored in memory 92 may in fact be hosted on computers that are external to computer system 100 but that can be electronically accessed by the computer system 100 over an Internet, intranet, or other form of network or electronic cable using network interface 54. In some embodiments, the computer system 100 makes use of models that are run from the memory associated with one or more graphical processing units in order to improve the speed and performance of the system. In some alternative embodiments, the computer system 100 makes use of models that are run from memory 92 rather than memory associated with a graphical processing unit.

The memory 92 of the computer system 100 stores:
an optional operating system 102 that includes procedures for handling various basic system services;
an analysis module 104 for determining whether a subject has a positive or negative molecular residual disease status for a cancer condition;
a first training dataset 106 that comprises:
   an electronic representation of a plurality of circulating tumor fragments (e.g., 108-1, 108-N; where N is a positive integer of 2 or greater), and
   for each respective circulating tumor DNA (ctDNA) fragment 108 in a plurality of ctDNA fragments, one or more sequence reads (e.g., 110-1-1, . . . , 110-1-M, where M is a positive integer) that were obtained from sequencing the respective ctDNA fragment 108 in a first sequencing reaction;
a first reference sequence 110 and
   for each respective reference region 112 in a plurality of reference regions (112-1, . . . , 112-Q, where Q is a positive integer of 2 or greater) of the first reference sequence, a number of circulating-tumor DNA (ctDNA) fragments 114 (e.g., 114-1, . . . , 114-Q) mapping to the respective reference region, a distribution 116 (e.g., 116-1, . . . , 116-Q) for the respective reference region, and an expected number of noise fragments 118 (e.g., 118-1, . . . , 118-Q) for the respective reference region;
an excess fragments per million value 120;
a corrected fragments per million value 122;
a second data set 124 that comprises:
   a plurality of cell-free fragments 126 (e.g., 126-1, . . . , 126-Z, where Z is a positive integer), and for each respective cell-free fragment 126, a corresponding one or more sequence reads 127 (e.g., 127-1-1, . . . , 127-1-X, where X is a positive integer) that were obtained from sequencing the respective cell-free fragment 126 in a second sequencing reaction; and
a candidate somatic variant set 128 comprising a plurality of candidate somatic variants (e.g., 130-1, . . . , 130-P, where P is a positive integer).

In some embodiments, one or more of the above identified data elements or modules of the computer system 100 are stored in one or more of the previously mentioned memory devices, and correspond to a set of instructions for performing a function described above. The above identified data, modules or programs (e.g., sets of instructions) need not be implemented as separate software programs, procedures or modules, and thus various subsets of these modules may be combined or otherwise re-arranged in various implementations. In some implementations, the memory 92 optionally stores a subset of the modules and data structures identified above. Furthermore, in some embodiments the memory 92 stores additional modules and data structures not described above.

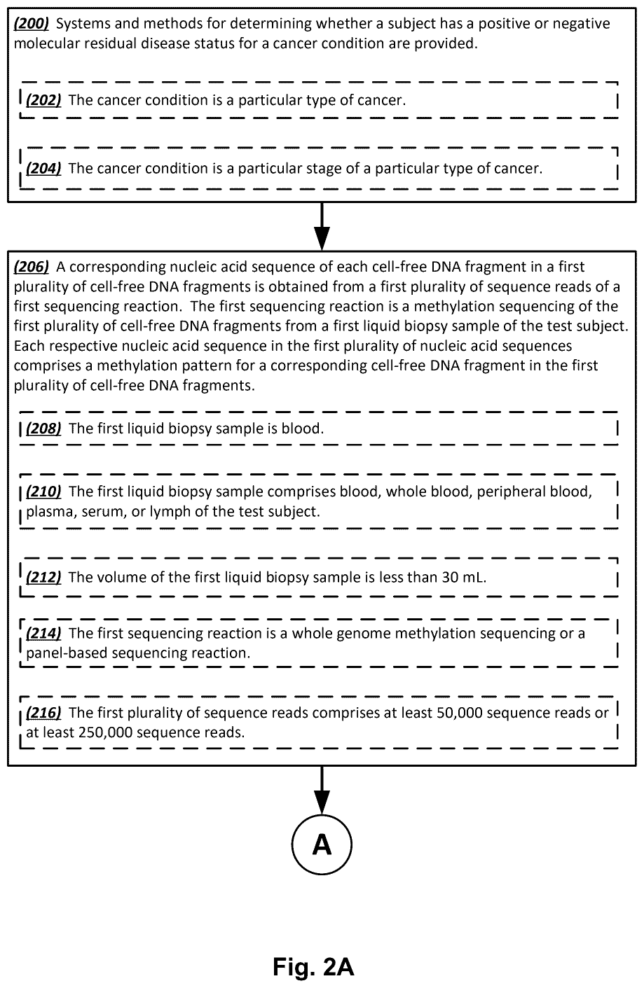

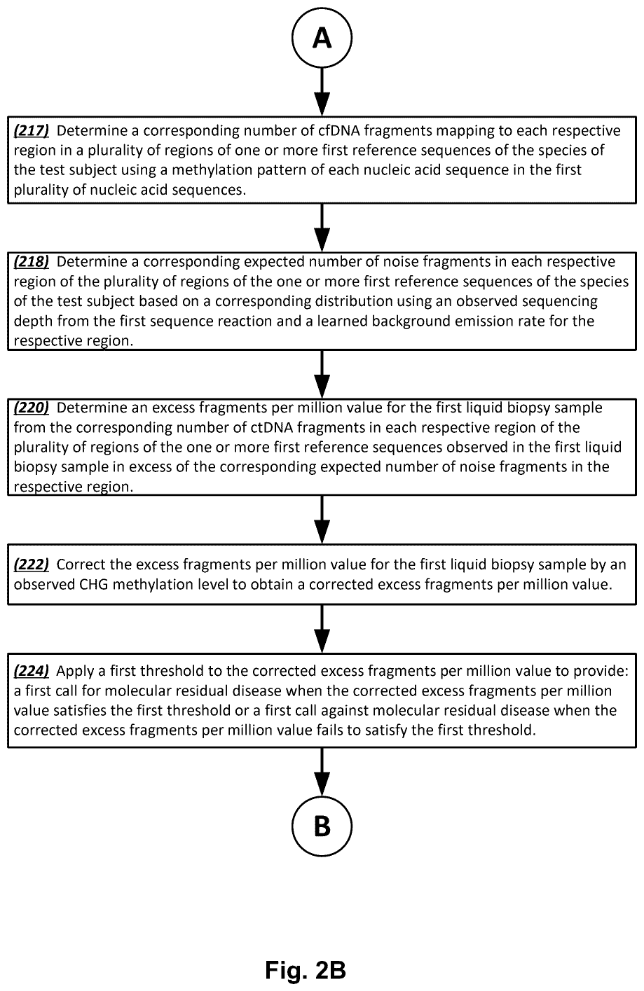

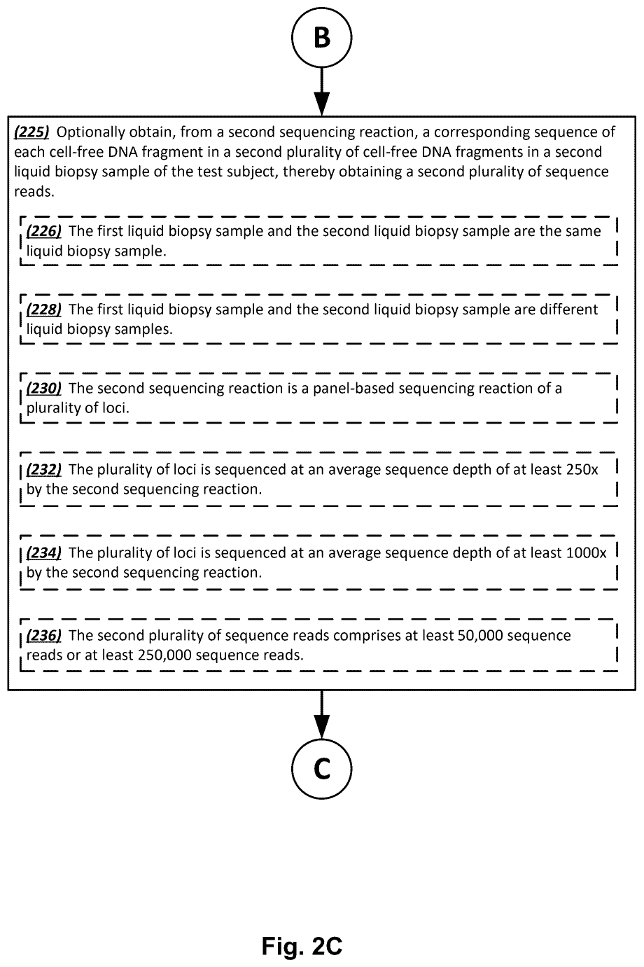

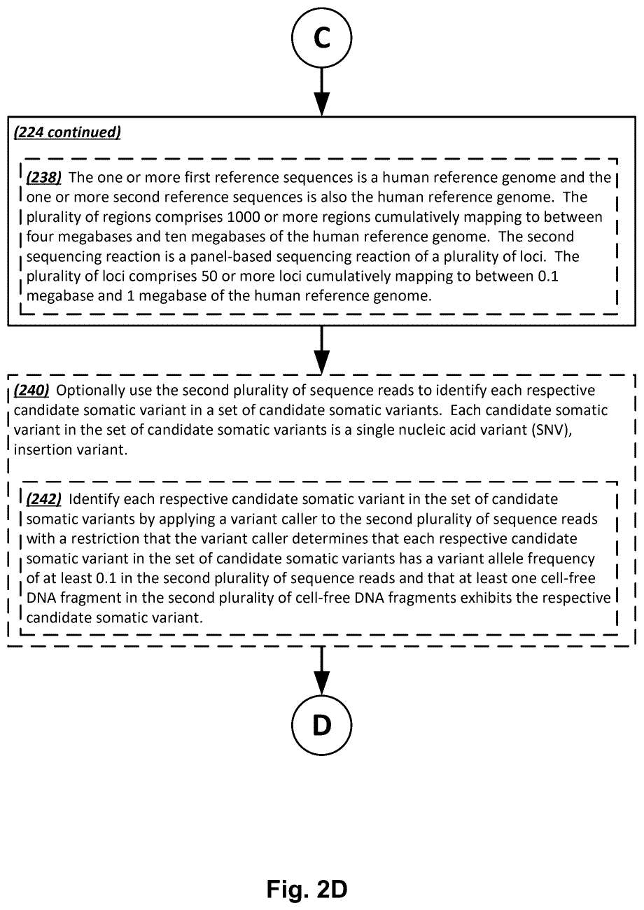

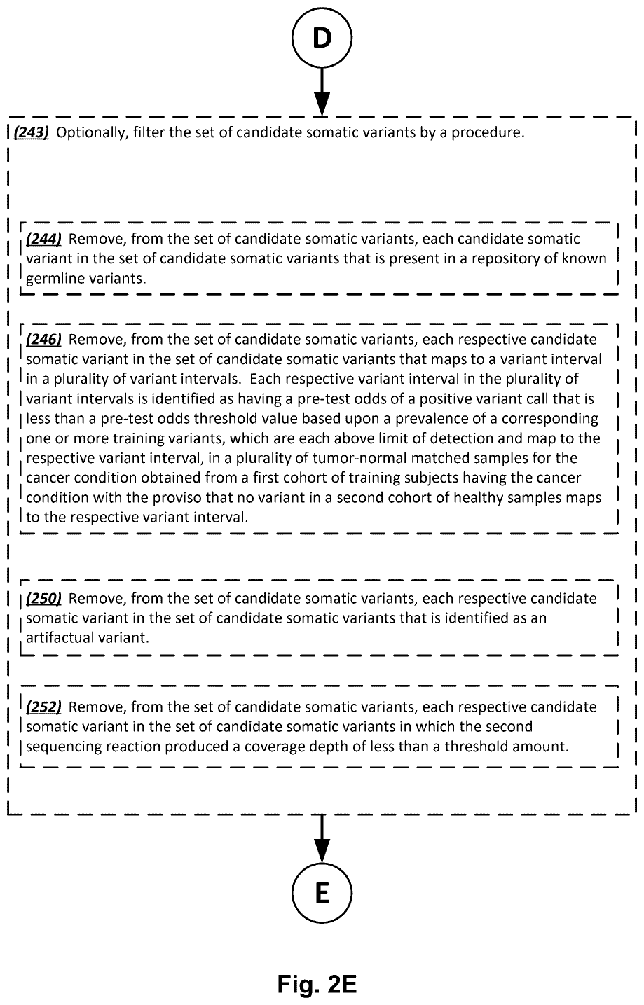

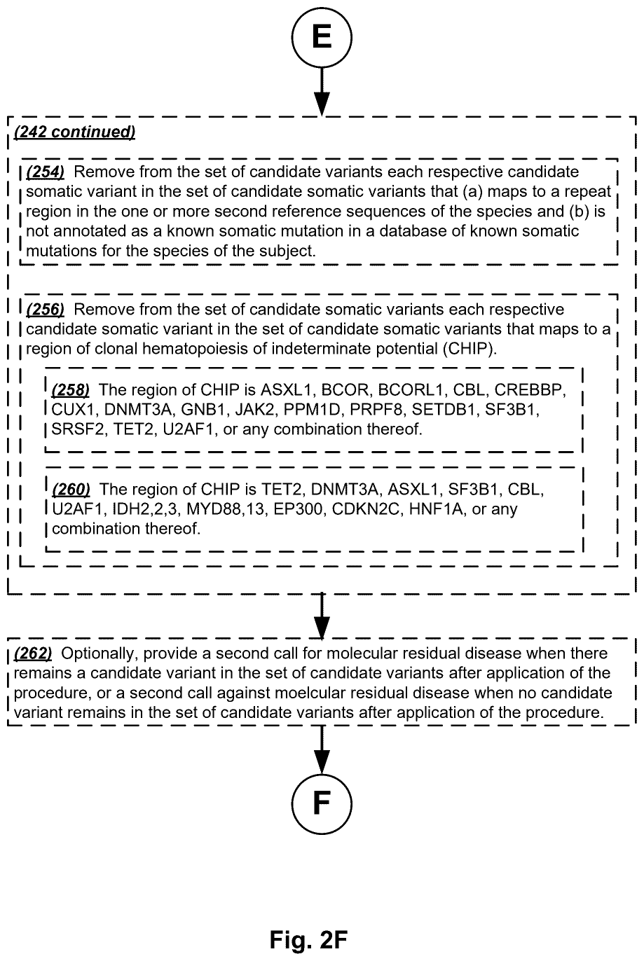

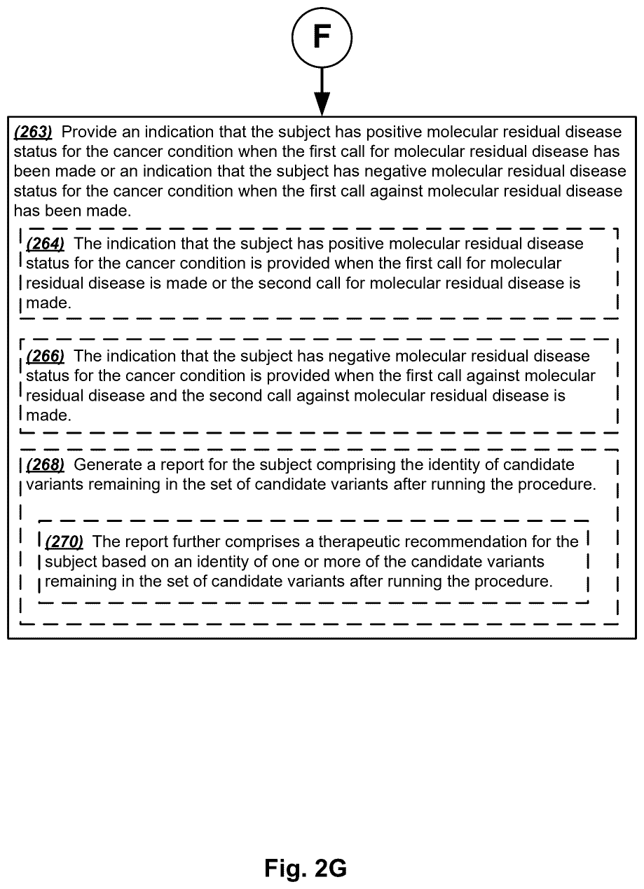

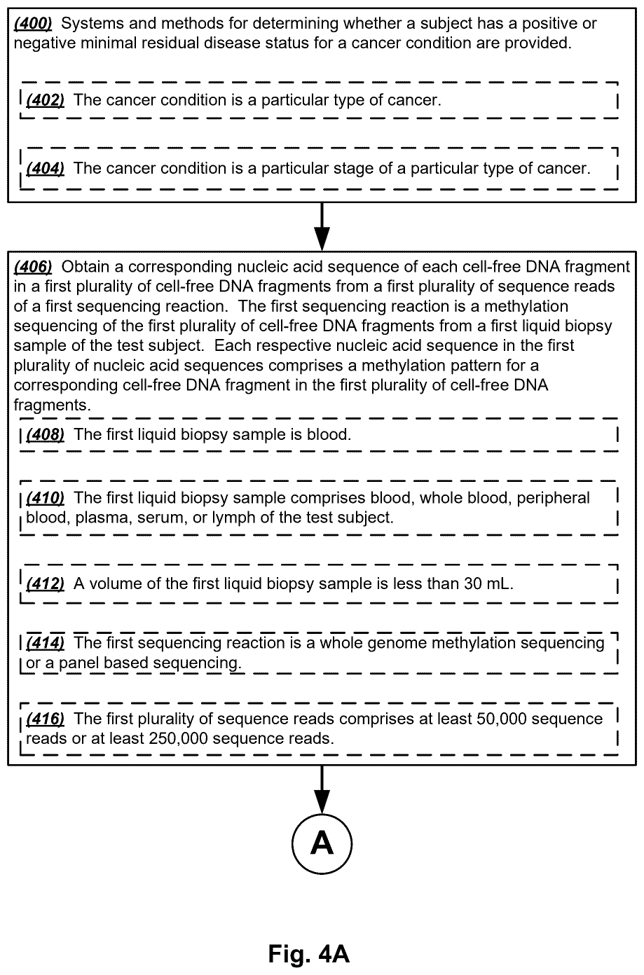

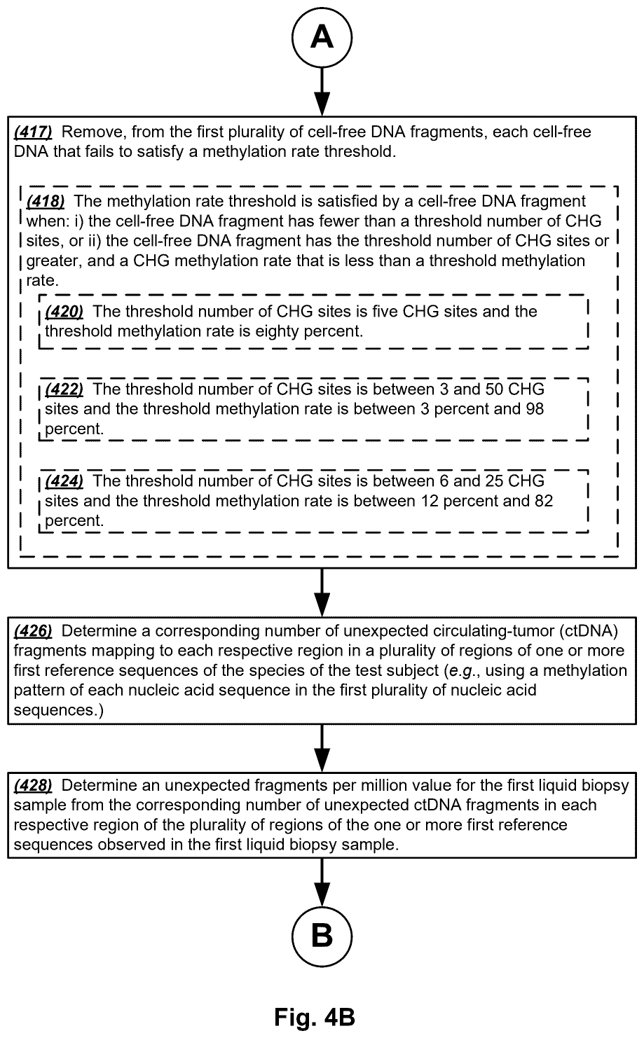

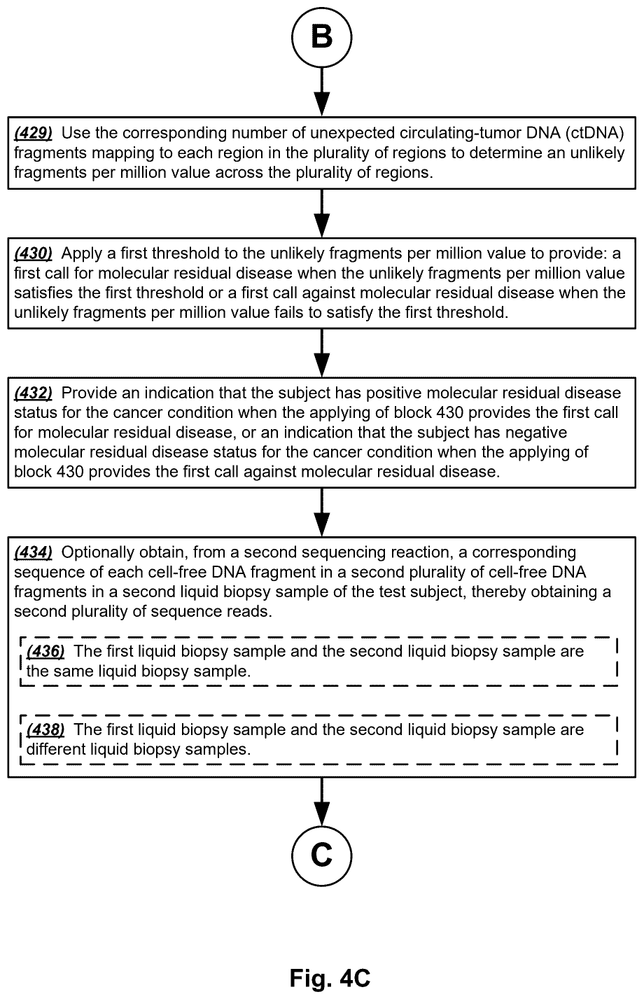

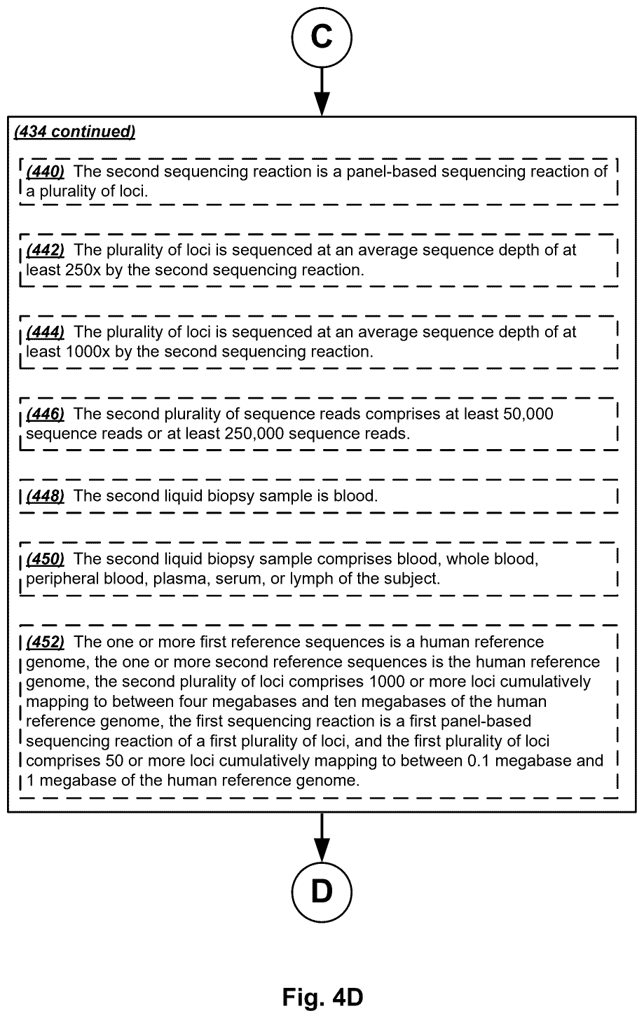

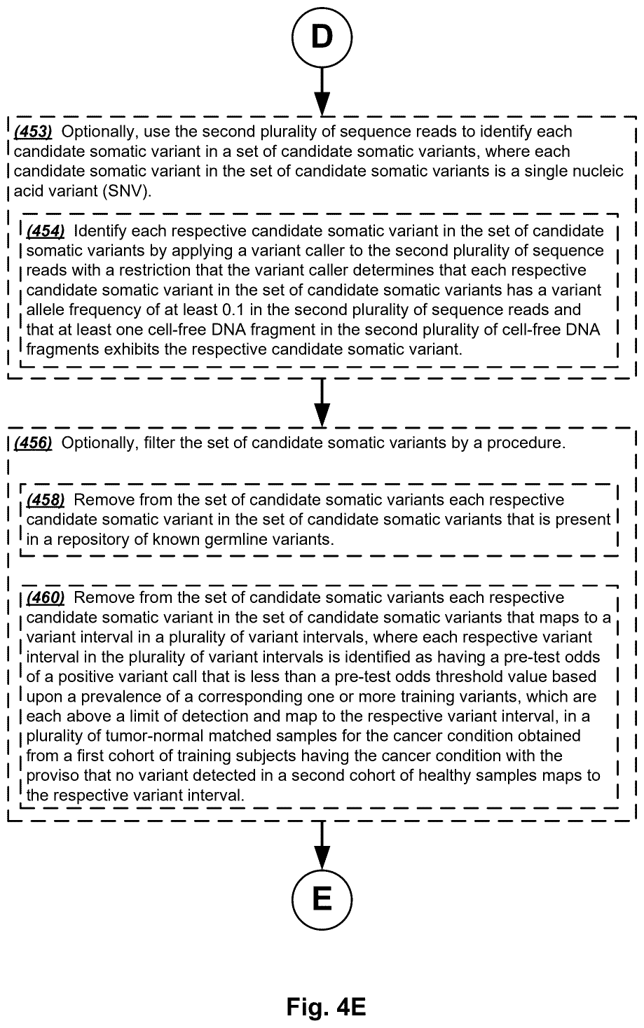

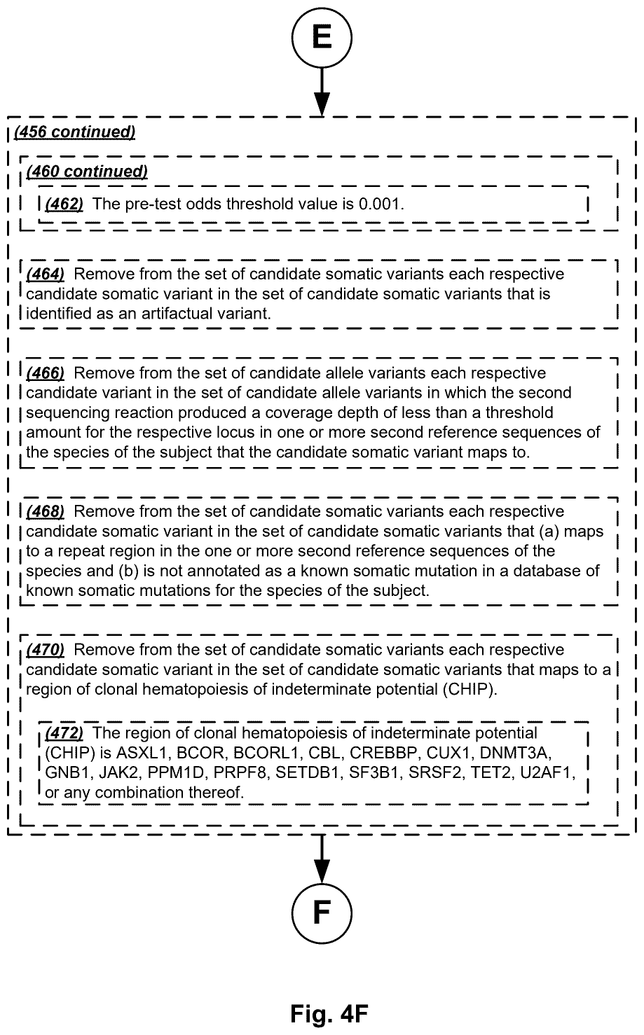

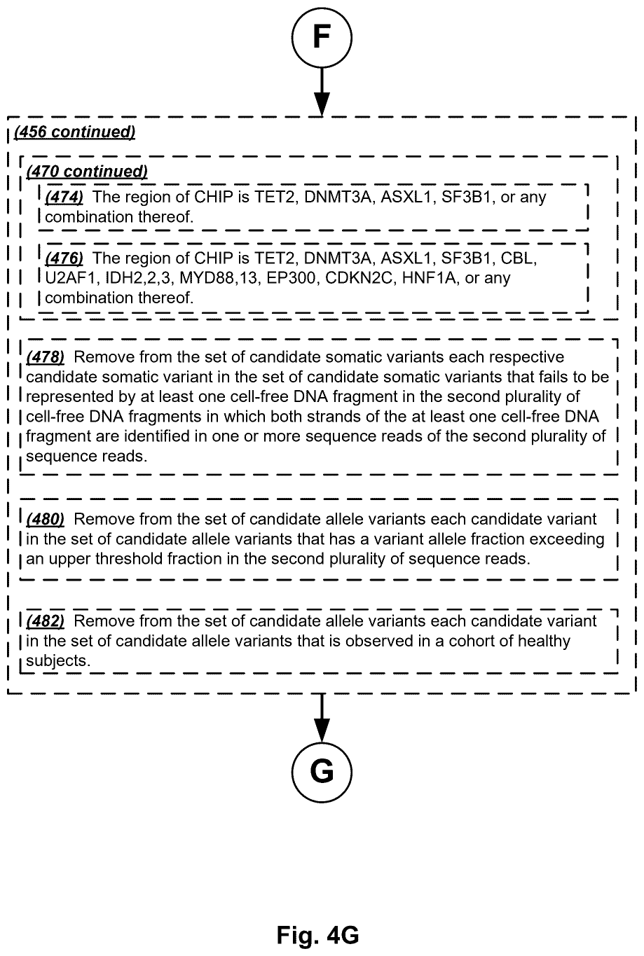

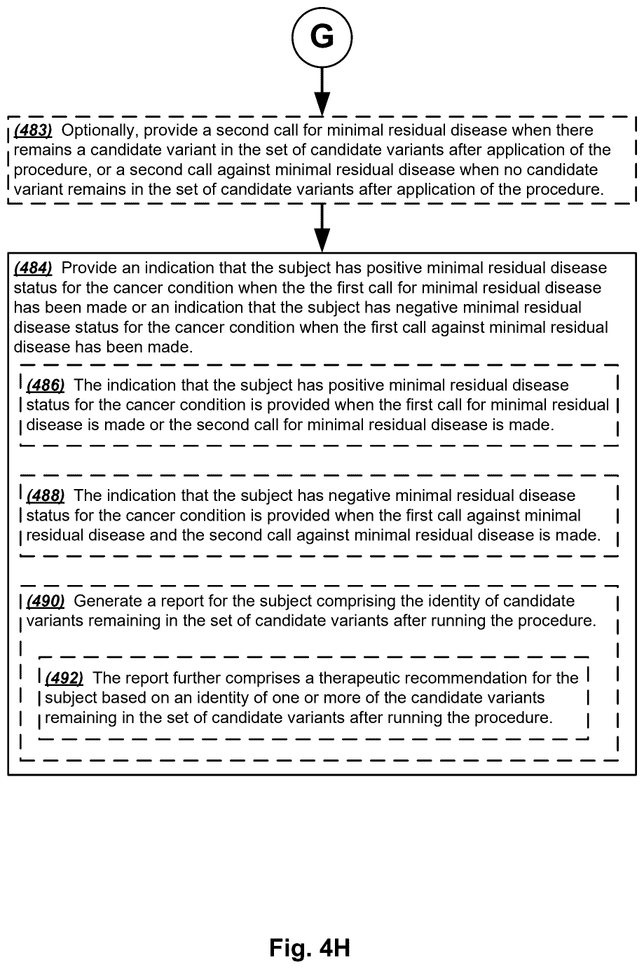

Now that a system for determining whether a subject has a positive or negative molecular residual disease status for a cancer condition has been disclosed, methods for performing such determinations are detailed with reference to FIG. 2 and FIG. 4 as discussed below.

### II. FIRST EMBODIMENT OF METHODS FOR DETERMINING WHETHER A SUBJECT HAS A POSITIVE OR NEGATIVE MOLECULAR RESIDUAL DISEASE STATUS FOR A CANCER CONDITION

Referring to block 200 of FIG. 2A systems and methods for determining whether a subject has a positive or negative molecular residual disease (MRD) status for a cancer condition is provided.

A cancer condition refers to a characteristic of a cancer patient's condition, e.g., a diagnostic status, a type of cancer, a location of cancer, a primary origin of a cancer, a cancer stage, a cancer prognosis, and/or one or more additional characteristics of a cancer (e.g., tumor characteristics such as morphology, heterogencity, size, etc.). In some embodiments, one or more additional personal characteristics of the subject are used to further describe the cancer state or cancer condition of the subject, e.g., age, gender, weight, race, personal habits (e.g., smoking, drinking, diet), other pertinent medical conditions (e.g., high blood pressure, dry skin, other diseases, etc.), current medications, allergies, pertinent medical history, current side effects of cancer treatments and other medications, etc.

Referring to block 202, in some embodiments, the cancer condition is a particular type of cancer. In some embodiments, the cancer condition is lung cancer (e.g., non-small-cell lung cancer). In some embodiments, the cancer condition is breast cancer. Additional non-limiting examples of cancer conditions include ovarian cancer, cervical cancer, uveal melanoma, colorectal cancer, chromophobe renal cell carcinoma, liver cancer, endocrine tumor, oropharyngeal cancer, retinoblastoma, biliary cancer, adrenal cancer, neural cancer, neuroblastoma, basal cell carcinoma, brain cancer, non-clear cell renal cell carcinoma, glioblastoma, glioma, kidney cancer, gastrointestinal stromal tumor, medulloblastoma, bladder cancer, gastric cancer, bone cancer, thymoma, prostate cancer, clear cell renal cell carcinoma, skin cancer, thyroid cancer, sarcoma, testicular cancer, head and neck cancer (e.g., head and neck squamous cell carcinoma), meningioma, peritoneal cancer, endometrial cancer, pancreatic cancer, mesothelioma, esophageal cancer, small cell lung cancer, HER2 negative breast cancer, ovarian serous carcinoma, HR+ breast cancer, uterine serous carcinoma, uterine corpus endometrial carcinoma, gastroesophageal junction adenocarcinoma, gallbladder cancer, chordoma, and papillary renal cell carcinoma.

Referring to block 204, in some embodiments, the cancer condition is a particular stage of a particular type of cancer. In some such embodiments, the stage of the particular type of cancer is a stage of cancer the subject was diagnosed with prior to treatment. Cancer is typically staged to determine the extent of its spread and to guide treatment decisions. The stage of cancer refers to the extent to which it has grown and spread from its original location. Each cancer has its own criteria for determine stage but generally relies on a determination of the size of the primary tumor (T) and whether it has invaded nearby tissues, evaluation of lymph node involvement (N) to find indications of whether cancer has spread to nearby lymph nodes, and assessment of distant metastasis (M), which indicates whether the cancer has spread to distant organs or tissues. Metastasis means that cancer has spread from the primary site to other parts of the body. In some embodiments the staging system used is the cancer TNM system, which combines the T, N, and M information to assign a stage. In some embodiments the stages are denoted using Roman numerals (I, II, III, IV) and may have subcategories (e.g., stage IIA, stage IIB) to provide more precise information. In a brief overview of these stages, in stage 0, the cancer is in situ, meaning it is confined to the layer of cells where it began and has not invaded nearby tissues, in stage I: the cancer is localized and small in size, in stage II, the cancer may be larger and/or have spread to nearby lymph nodes, but it is still relatively localized, in stage III, the cancer has typically spread further into nearby tissues and may involve more lymph nodes, in stage IV, the cancer has spread to distant organs or tissues, indicating metastasis. This is often the most advanced stage. The specific criteria for each stage can vary depending on the type of cancer. See, for example, details of TNM staging for breast cancer in Part et al., 2011, “Clinical relevance of TNM staging system according to breast cancer subtypes,” Annals of Oncology 22 (7), pp. 1554-1560, which is hereby incorporated by reference. Additionally, some cancers have their own staging systems tailored to their characteristics. See the Internet at cancer.gov/about-cancer/diagnosis-staging/staging.

Referring to block 206, a corresponding nucleic acid sequence of each cell-free DNA fragment 108 in a first plurality of cell-free DNA fragments is obtained (e.g., in the form of a first data set 106) from a first plurality of sequence reads 110 of a first sequencing reaction. The first sequencing reaction is a methylation sequencing of the first plurality of cell-free DNA fragments from a first liquid biopsy sample of the test subject. Each respective nucleic acid sequence in the first plurality of nucleic acid sequences is accompanied by a methylation pattern for a corresponding cell-free DNA fragment in the first plurality of cell-free DNA fragments.

In some embodiments, the cell-free DNA is fragmented for the first sequencing reaction. In some such embodiments, the first sequencing reaction of block 206 uses paired-end sequencing so that there are two corresponding sequence reads in the first plurality of sequence reads that encapsulate the sequencing data for each respective cell-free DNA fragment in the first plurality of cell-free DNA fragments.

In some embodiments, personal data corresponding to the second and a record of the one or more biological samples obtained (e.g., patient identifiers, patient clinical data, sample type, sample identifiers, cancer conditions, etc.) are also obtained.

Referring to block 208, in some embodiments, the first liquid biopsy sample is blood. Referring to block 210, in some embodiments, the first liquid biopsy sample comprises blood, whole blood, peripheral blood, plasma, serum, or lymph of the test subject. In some embodiments, one or more of the biological samples obtained from the patient are a biological liquid sample, also referred to as a liquid biopsy sample. In some embodiments, one or more of the biological samples obtained from the patient are selected from blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. In some embodiments, the liquid biopsy sample includes blood and/or saliva. In some embodiments, the liquid biopsy sample is peripheral blood. In some embodiments, one or more blood samples are collected from a subject in commercial blood collection containers. In some embodiments, saliva samples are collected from patients in commercial saliva collection containers.

Referring to block 212, in some embodiments, the volume of the first liquid biopsy sample is less than 30 mL. In some embodiments, the volume of the liquid biopsy sample is from 1 mL to 50 mL, from 2 mL to 40 mL, from 3 mL to 35 mL, or from 5 mL to 31 mL. For example, in some embodiments, the liquid biopsy sample has a volume of about 1 mL, about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, or greater.

Liquid biopsy samples include cell free nucleic acids, including cell-free DNA (cfDNA). As described above, cfDNA isolated from cancer patients includes DNA originating from cancerous cells, also referred to as circulating tumor DNA (ctDNA), cfDNA originating from germline (e.g., healthy or non-cancerous) cells, and cfDNA originating from hematopoietic cells (e.g., white blood cells). The relative proportions of cancerous and non-cancerous cfDNA present in a liquid biopsy sample varies depending on the characteristics (e.g., the type, stage, lineage, genomic profile, etc.) of the patient's cancer.

In some embodiments cell-free DNA is isolated from the liquid biological sample using commercially available reagents, including digestion with proteinase K. In some embodiments, the selective binding properties of a silica membrane are used to extract cell-free DNA from the first liquid biological sample using circulating nucleic acid kits. In some such embodiments, the liquid biological sample is lysed in an optimized buffer and adjusted to binding conditions. Then, the liquid biological sample is loaded directly onto a spin column. In this step, cell-free DNA is bound to the silica membrane, and contaminants are removed in wash steps. Finally, pure cell-free DNA is eluted in small volumes of a low-salt buffer for downstream applications. See, Hai et al., 2022, “Whole-genome circulating tumor DNA methylation landscape reveals sensitive biomarkers of breast cancer,” MedComm (2020) September 3 (3):e134, which is hereby incorporated by reference.

In some embodiments, adapters such as unique dual index (UDI) adapters are ligated onto the cell-free DNA fragments. In some embodiments, adapters with unique molecular indices (UMI), which are short nucleic acid sequences (e.g., 4-10 base pairs), are ligated onto the cell-free DNA fragments. In some embodiments, the UDI adapters include UMIs. In some embodiments, UMIs are degenerate base pairs that serve as a unique tag that can be used to identify sequence reads originating from a specific DNA fragment. In some embodiments, the adapters are protected against methyl conversion and will not be affected by a methyl conversion step in a wet lab protocol. In some embodiments, e.g., when multiplex sequencing will be used to sequence cell-free DNA fragments from a plurality of samples (e.g., from the same or different subjects) in a single sequencing reaction, a patient-specific index is also added to the nucleic acid molecules. In some embodiments, the sample specific index is a short nucleic acid sequence (e.g., 3-20 nucleotides) that are added to ends of cell-free DNA fragments during library construction, that serve as a unique tag that can be used to identify sequence reads originating from a specific patient sample.

In some embodiments, an adapter includes a PCR primer landing site, designed for efficient binding of a PCR or second-strand synthesis primer used during the sequencing reaction. In some embodiments, an adapter includes an anchor binding site, to facilitate binding of the cell-free DNA fragment to anchor oligonucleotide molecules on a sequencer flow cell, serving as a seed for the sequencing process by providing a starting point for the sequencing reaction. During PCR amplification following adapter ligation, the UMIs, patient indexes, and binding sites are replicated along with the attached cell-free DNA fragment. This provides a way to identify sequence reads that came from the same original cell-free DNA fragment in downstream analysis.

In some embodiments the sequence reads in the first plurality of sequence reads are trimmed to remove sequencing adapters, amplification primers, and low-quality bases in read ends.

Referring to block 214, in some embodiments, the first sequencing reaction is a whole genome methylation sequencing or targeted panel sequencing. Regardless of whether a targeted, probe based methylation sequencing or a whole genome methylation sequencing is performed, in some embodiments nucleic acids isolated from the biological sample (e.g., cfDNA) are treated to convert unmethylated cytosines to uracils.

In some embodiments, when the nucleic acids are sequenced, cytosines called in the sequencing reaction are methylated, since the unmethylated cytosines are converted to uracils and accordingly would be called as thymidines, rather than cytosines, in the sequencing reaction. In some embodiments commercial kits are used for bisulfite-mediated conversion of methylated cytosines to uracils in the first sequencing reaction. In some embodiments commercial kits are used for enzymatic conversion of methylated cytosines to uracils in the first sequencing reaction.

Some embodiments of the first sequencing reaction of block 206 employ the NEB protocol of the NEBNExt enzymatic methyl-seq kit (New England Biolabs, Ipswich, Massachusetts). In some such embodiments, an enzymatic methyl conversion process that oxidizes 5-methylcytosines (5mC) and 5-hydroxymethylcytosines (5hmC) is performed in the first sequencing reaction of block 206. This reaction protects modified cytosines from downstream deamination. TET2 enzymatically oxidizes 5mC and 5hmC through a cascade reaction into 5-carboxycytosine (5-methylcytosine (5mC)=>5-hydroxymethylcytosine (5hmC)=>5-formylcytosine (5fC)=>5-carboxycytosine (5caC)).

In some alternative embodiments, 5hmC is protected from deamination by glucosylation to form 5ghmc using the oxidation enhancer. In some embodiments of the first sequencing reaction of block 206, APOBEC is used to enzymatically deaminate unmodified cytosines to uracils, but does not convert 5caC and 5gmhC. During amplification, uracils are converted to thymines.

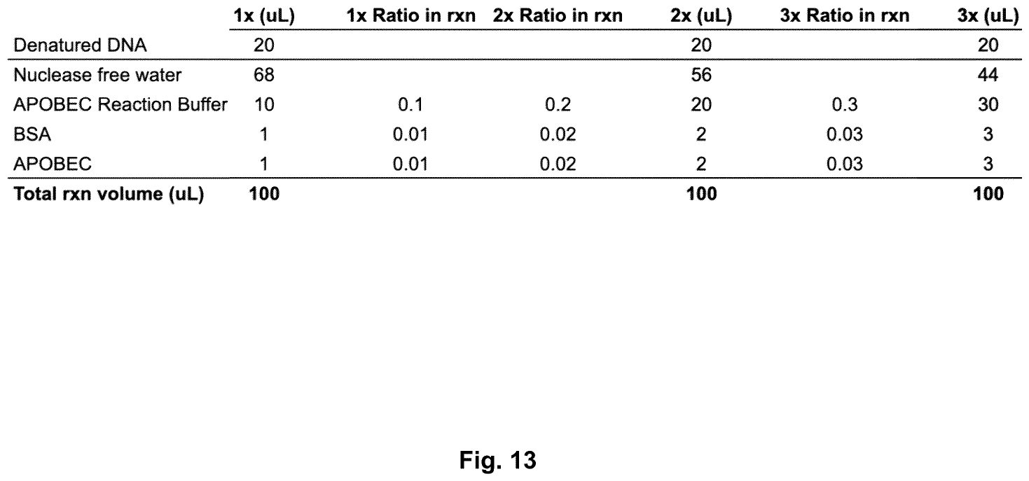

In some embodiments, an increased enzymatic ratio in the reaction and/or incubation time, relative to the NEB protocol discussed above, is used to improve the percent of deaminated cytosines in the first sequencing reaction of block 206. For instance, in some embodiments, different ratios of the APOBEC mix (enzyme, buffer & BSA) are used in the first sequencing reaction of block 206. With the published NEB protocol termed 1×, in some embodiments 2× or 3× ratios are used instead as outlined in FIG. 13. In some embodiments a 1.5× or 3.5× ratios is used. In some embodiments an A.Bx ratio is used where A is a real number selected from the range of between 1 and 5 and B is a real number selected from the range between 1 and 25 with the proviso that B is greater than A.

In some embodiments, the cell-free DNA fragments are amplified and purified using commercial reagents. In some such embodiments, the concentration and/or quantity of the cell-free DNA fragments are quantified using a fluorescent dye and a fluorescence microplate reader, standard spectrofluorometer, or filter fluorometer. In some embodiments, library amplification is performed on a device (e.g., an Illumina C-Bot2) and the resulting flow cell containing amplified cell-free DNA fragments is sequenced. In some embodiments, the deamination reaction is incubated at 37° C. for three hours followed by a hold at 4° C. In some embodiments this incubation time is doubled in order to improve methyl conversion efficiency. In some embodiments, all enzymatic ratios listed in FIG. 13 include a 4-hour, 5-hour, 6-hour, 7-hour, 8-hour, or 9-hour incubation.

In some embodiments sequencing is performed on a next generation sequencer (e.g., an Illumina HiSeq 4000, Illumina NovaSeq 6000, Oxford Nanopore, Biomodal) to a unique on-target depth selected by the user. In some embodiments, sequencing is performed using sequencing-by-synthesis technology (Illumina), pyrosequencing (454 Life Sciences), ion semiconductor technology (Ion Torrent sequencing), single-molecule real-time sequencing (Pacific Biosciences), or sequencing by ligation (SOLID sequencing).

In some embodiments the first sequencing reaction of block 206 is a panel based sequencing reaction.

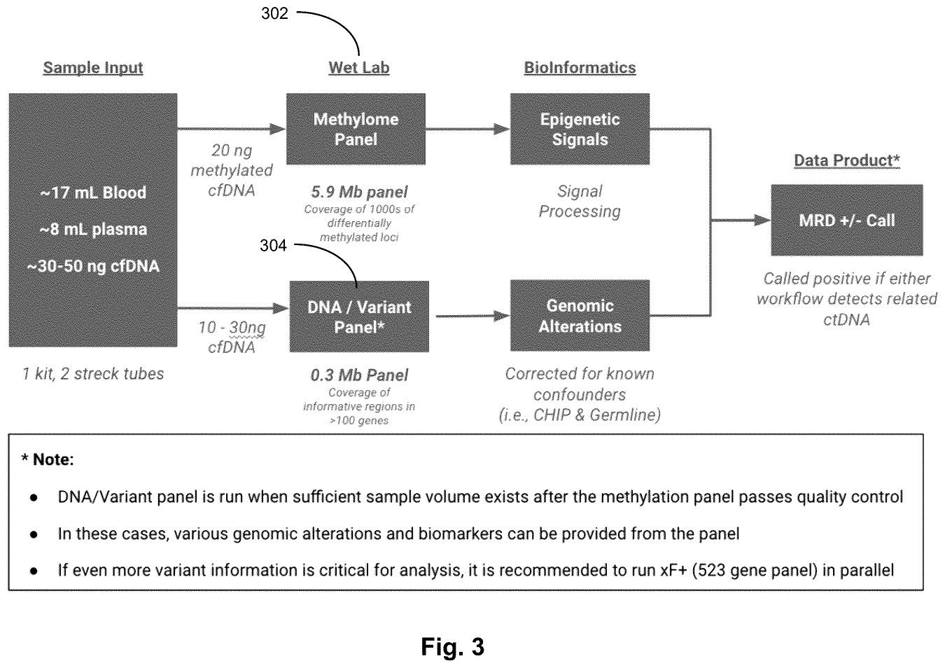

In some embodiments the first sequencing reaction of block 206 is the panel based sequencing reaction illustrated as sequencing reaction 302 in FIG. 3. In some embodiments the first sequencing reaction of block 206 is a panel based sequencing reaction that makes use of a panel described in Example 6.

In some embodiments the first sequencing reaction of block 206 includes probes for 100 or more, 200 or more, 500 or more, 1000 or more, 1500 or more 2000 or more, 2500 or more, 3000 or more, 3500 or more, 4000 or more, 5000 or more or 10,000 or more differentially methylated loci.

In some embodiments the first sequencing reaction of block 206 includes a plurality of probes. In some embodiments, the plurality of probes is one described in Example 6. In some embodiments, the plurality of loci is one described in Example 6. In some embodiments the plurality of probes includes all four potential sequences at a given loci: methylated, unmethylated, sense and antisense. In some embodiments the plurality of probes includes a first subset that capture methylated sequences for a plurality of loci and a second subset that capture unmethylated sequences for the same plurality of loci. In some embodiments the number of probes in the first subset is equal to the number of probes in the second subset. In alternative embodiments, while the first and second subset of probes each collectively map to the same plurality of loci, the ratio of the number of probes in the first subset to the number of probes in the second subset is other than 1:1. That is, they are mixed at different ratios with emphasis on probes capturing methylated sequences. For instance, in some embodiments there are more probes in the first subset (probes that capture methylated sequences). In some embodiments, the ratio of probes in the first subset to probes in the second subset is 1.25 to 1.00, 1.50 to 1.00, 1.75 to 1.00, 2.00 to 1.00, or 3.00 to 1.00. In some embodiments, the ratio of probes in the first subset to probes in the second subset is X to Y, where X and Y are real positive numbers and X is greater than Y. Without intending to be limited by any particular theory, increasing the concentration of probes in such embodiments enhances methylation detection, reduces the noise from unmethylated normal sequences, and improves assay sensitivity.

Referring to block 216 of FIG. 2A, in some embodiments, the first plurality of sequence reads comprises at least 50,000 sequence reads or at least 250,000 sequence reads. In some embodiments the first plurality of sequence reads comprises at least 1000, at least 2000, at least 3000, at least 4000, at least 5000, at least 6000, at least 7000, at least 8000, at least 9000, at least 10,000, at least 50,000, at least 100,000, at least 500,000, at least 1 million, at least 2 million, at least 3 million, at least 4 million, at least 5 million, at least 6 million, at least 7 million, at least 8 million, at least 9 million, or more sequence reads. In some embodiments, the first plurality of sequence reads consists of between 50,000 sequence reads and 10 million sequence reads. In some embodiments, the first plurality of sequence reads consists of between 100,000 sequence reads and 8 million sequence reads. In some embodiments, the first plurality of sequence reads consists of between 200,000 sequence reads and 6 million sequence reads.

In some embodiments, the quality of the sequence reads is evaluated (e.g., by interrogating quality metrics like Phred score, base-calling error probabilities, Quality (Q) scores, and the like) and/or removing sequence reads that do not satisfy a threshold quality (e.g., an inferred base call accuracy of at least 80%, at least 90%, at least 95%, at least 99%, at least 99.5%, at least 99.9%, or higher). In some embodiments, the first plurality of sequence reads is filtered based on one or more properties, e.g., removing sequence reads from the first plurality of sequence reads that fail to satisfy a lower or upper size threshold or removing duplicate sequence reads.

In some embodiments the first plurality of cell-free DNA fragments is optionally filtered to remove each cell-free DNA fragment that fails a CHG context threshold and/or fails to satisfy a methylation rate threshold.

CHG methylation refers to a methylated cytosine that occurs within a CHG trinucleotide context, where H is the IUPAC symbol representing any non-guanine nucleotide (i.e., A, C, or T). In humans, cytosine methylation almost exclusively occurs within CpG contexts, and not within CHG contexts. However, methylated cytosines are still observed within CHG contexts in human sequencing data due to enzymatic failures, such as during NEBNext Enzymatic Methyl-Seq (EM-Seq) library preparation processes. For example, in some instances where the APOBEC enzyme is used for methylation sequencing, the APOBEC enzyme fails to properly deaminate an unmethylated cytosine into a uracil, that cytosine nucleotide is read as a “C” during sequencing, which causes it to be erroneously classified as a methylated cytosine. Though this failure mode is easiest to identify among CHG contexts, which are not naturally methylated in humans, APOBEC failure can occur in any genomic context, including CpGs.

While methylation sequencing errors, such as APOBEC failure, at any grain is considered undesirable, it may especially confound the models of the present disclosure when they lead to fragment level failures. Fragment-level APOBEC failure represents the case in which cytosines of any context on a fragment appear methylated due to complete or near-complete failure of the APOBEC enzyme during the enzymatic conversion step for that fragment. This is particularly confounding because, in one embodiment, the signal used in the present disclosure is derived from fragments that exhibit high degrees of CpG methylation among the differentially methylated regions (DMRs) specified by the model. Consequently, a fragment that presents with many methylated CpGs is equally likely to be classified as tumor-derived whether the observed hypermethylation occurred as a product of legitimate biological processes or fragment-level methylation sequencing failure.

To mitigate the risk of artifactually methylated fragments contributing to the signal used in the present disclosure, some embodiments include a pre-processing filter that specifically targets and removes from the first plurality of cell-free DNA fragments “unconverted fragments,” that is fragments that have experienced widespread unmethylation cytosine deamination failure. Because CpG methylation can be both biologically or artifactually-derived in humans, the rate at which CpG methylation is observed (methylation rate) on a given fragment may not say anything decisive about the presence or absence of unmethylation cytosine deamination failure. The term “methylation rate” in the context of CHG methylation refers to the frequency or proportion of cytosines in the CHG context that are methylated in a given DNA fragment. Given that CHG methylation does not occur naturally in humans, in most circumstance, and can hence be confidently deemed an artifact, unconverted fragment identification relies entirely on the rate at which CHG methylation is observed on a DNA fragment (methylation rate).

In some embodiments, fragments that contain five or more CHG contexts and exhibit CHG methylation rates in excess of 80% are deemed “unconverted,” or likely to have been seriously affected by methylation sequencing failure, such as APOBEC failure. Accordingly, in some embodiments these cell-free DNA fragments are removed from the first plurality of cell-free DNA fragments prior to further processing. In some embodiments this both reduces confounding factors presented to the models of the present disclosure and/or mitigates the risk of artifactual signal generation.

In some embodiments, the threshold number of CHG sites is five CHG sites and the threshold methylation rate is eighty percent. In some embodiments, the threshold number of CHG sites is between 3 and 50 CHG sites and the threshold methylation rate is between 3 percent and 98 percent. In some embodiments, the threshold number of CHG sites is between 6 and 25 CHG sites and the threshold methylation rate is between 12 percent and 82 percent.

In some embodiments the threshold methylation rate is between 12 percent and 82 percent. In some embodiments the threshold methylation rate is between 5 percent and 95 percent. In some embodiments the threshold methylation rate is between 5 percent and 10 percent, between 10 percent and 15 percent, between 15 percent and 20 percent, between 20 percent and 25 percent, between 25 percent and 30 percent, between 30 percent and 35 percent, between 35 percent and 40 percent, between 40 percent and 45 percent, between 45 percent and 50 percent, between 50 percent and 55 percent, between 55 percent and 60 percent, between 60 percent and 65 percent, between 65 percent and 70 percent, between 70 percent and 75 percent, between 75 percent and 80 percent, between 80 percent and 85 percent, between 85 percent and 90 percent, or between 90 percent and 95 percent. In some embodiments, the threshold methylation rate is 5, 10, 15, 20, 25, 30, 35, 40, 45, 50, 55, 60, 65, 70, 75, 80, 85, 90, or 95 percent.

In some embodiments cell-free DNA fragment are removed from the first plurality of cell-free DNA fragments prior to execution of blocks subsequent to block 216 in FIG. 2. For example, in some embodiments, cell-free DNA fragments that fail to satisfy a methylation rate threshold specified below in block 417, block 418, block 420, block 422, and/or block 422, are removed from the first plurality of cell-free DNA fragments. Nonlimiting examples of methylation rate thresholds for these blocks are illustrated in FIG. 4B. In some embodiments, cell-free DNA fragments that contain five or more CHG contexts and exhibit CHG methylation rates in excess of 80% are deemed “unconverted,” or likely to have been seriously affected by methylation sequencing failure, such as APOBEC failure. Accordingly, in some embodiments these cell-free DNA fragments are removed from the first plurality of cell-free DNA fragments prior to further processing to both reduce confounding factors presented to the models of the present disclosure and to mitigate the risk of artifactual signal generation. In some embodiments, the methylation rate threshold is satisfied by a cell-free DNA fragment when the cell-free DNA fragment has at least one CHG site that is methylated.

Referring to block 217, in some embodiments, a determination of a corresponding number of circulating tumor DNA fragments 108 mapping to each respective region 112 in a plurality of regions of one or more first reference sequences 110 of the species of the test subject is made using a methylation pattern of each nucleic acid sequence in the first plurality of nucleic acid sequences.

Defining the plurality of regions. To perform block 217, the plurality of regions of the one or more first reference sequences is defined. In some embodiments, the one or more first reference sequences is a single reference sequence, such as a reference human genome, and each region in the plurality of regions is a particular locus in the reference human genome. A nonlimiting example of a reference human genome is the human reference genome (hg19). In some embodiments, the one or more first reference sequences is a single reference sequence, such as a reference human genome, and each region in the plurality of regions is a particular locus in the reference human genome that is known to exhibit differential CpG methylation between a particular cancer condition versus a cancer free condition.

In some embodiments the one or more first reference sequences of the species of the test subject refers to any sequenced or otherwise characterized genome, whether partial or complete, of any organism or pathogen that may be used to reference identified sequences from a subject. Typically, a reference genome is derived from a subject of the same species as the subject whose sequences are being evaluated. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or pathogen, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38). For a haploid genome, there can be only one nucleotide at each locus. For a diploid genome, heterozygous loci can be identified; each heterozygous locus can have two alleles, where either allele can allow a match for alignment to the locus.

In some embodiments, the plurality of regions for block 217 comprises regions of the genome whose differential methylation state is associated with cancer. Example 6 provides one such example. More generally, Skvortsova, “The DNA methylation landscape in cancer,” Essays Biochem 63 (6), pp. 797-811, which is hereby incorporated by reference, discloses approaches for identifying such genomic regions and exemplary regions in humans. See also Biswas et al., 2017, “Epigenetics in cancer: Fundamentals and beyond,” Pharmacol. Ther. 173, pp. 118-134, which is hereby incorporated by reference.

In some embodiments the plurality of regions is determined in accordance with Example 2. In one instance in accordance with Example 2, the top 100 regions for differential signal were selected from the plurality of regions, and the remaining regions in the plurality of regions were not used to compute the excess fragments per million value in accordance with block 220 and the corrected excess fragments per million value in accordance with block 222. In another instance in accordance with Example 2, the top 250 regions for differential signal were selected from the plurality of regions and the remaining regions in the plurality of regions were not used to compute the excess fragments per million value in accordance with block 220 and the corrected excess fragments per million value in accordance with block 222. In other embodiments, the top N regions for differential signal are selected from the plurality of regions to compute the excess fragments per million value in accordance with block 220 and the corrected excess fragments per million value in accordance with block 222, while the remaining regions not selected in the plurality of regions were not used to compute the excess fragments per million value in accordance with block 220 and the corrected excess fragments per million value in accordance with block 222. In some such embodiments, N is 15, 25, 50, 100, 150, 250, 300, 350, or 400. In some such embodiments, N is any integer between 15 and 2000.

In some embodiments the plurality of regions consists of between 10 and 250 different regions of the one or more reference sequences. In some embodiments the plurality of regions consists of between 20 and 300 different regions of the one or more reference sequences. In some embodiments the plurality of regions comprises 10 or more regions, 30 or more regions, 40 or more regions, 60 or more regions, 80 or more regions, or 100 or more regions of the one or more reference sequences.

In some embodiments the plurality of regions consists of between 100 and 2500 different regions of the one or more reference sequences. In some embodiments the plurality of regions consists of between 200 and 3000 different regions of the one or more reference sequences. In some embodiments the plurality of regions comprises 100 or more regions, 300 or more regions, 400 or more regions, 600 or more regions, 800 or more regions or 10,000 or more regions of the one or more reference sequences.

In some embodiments the plurality of regions consists of between 1000 and 25,000 different regions of the one or more reference sequences. In some embodiments the plurality of regions consists of between 2000 and 30,000 different regions of the one or more reference sequences. In some embodiments the plurality of regions comprises 1000 or more regions, 3000 or more regions, 4000 or more regions, 6000 or more regions, 8000 or more regions or 10,000 or more regions of the one or more reference sequences.

Mapping cell-free DNA fragments to the plurality of regions. To perform block 217, each of the cell-free DNA fragments in the first plurality of cell-free DNA fragments are mapped onto the one or more reference sequences in order to ascertain which regions in the plurality of regions they overlap. In some embodiments in accordance with block 217, the cell-free nucleic acid sequences are mapped to the one or more first reference sequences of the species of the test subject using a mapping program.

In some embodiments in accordance with block 217, one or more alignment algorithms map the first plurality of cell-free DNA fragments to one or more first reference sequences, e.g., a reference genome, exome, or targeted-panel construct. Additional algorithms for mapping nucleic acid sequence to one or more first reference sequences are known in the art, for example, Burrows-Wheeler Alignment (BWA), Blat, SHRIMP, LastZ, and MAQ. In some embodiments cell-free DNA fragments are mapped to the one or more first reference sequences by mapping the sequence reads associated with such cell-free DNA fragments from the first sequencing reaction. In some embodiments cell-free DNA fragments are mapped to the one or more first reference sequences by mapping the sequences of the cell-free DNA fragments directly.

In some embodiments in accordance with block 217, the sequences of the first plurality of cell-free DNA fragments are mapped to the plurality of regions based on genomic coordinates.

In some embodiments, any cell-free fragment that overlaps any portion of any region 112 is mapped to that region. In such embodiments, the mapping occurs at the fragment context and not the site context.

In some embodiments, if regions are proximal to each other on the one or more first reference sequences and a single cell-free DNA fragment overlaps with multiple regions, that cell-free DNA fragment is mapped to each of the multiple regions.

Determining which of the mapped cell-free DNA fragments are circulating tumor fragments. To perform block 217, once each respective cell-free DNA fragment in the first plurality of cell-free DNA fragments has been mapped to a particular one or more regions in the plurality of regions, a model that makes use of the methylation pattern of the respective cell-free DNA fragment is used to predict whether the cell-free DNA fragment is a circulating tumor fragment or not. In other words, once the first plurality of cell-free DNA fragments has been mapped to the plurality of regions, the methylation pattern of each respective cell-free DNA fragment is used to assign a probability that the respective cell-free DNA fragment is ctDNA.

In some embodiments, the methylation pattern of a respective cell-free DNA fragment in the first plurality of cell-free DNA fragments that has been mapped to a given region in the plurality of regions is used to determine a methylation configuration of the respective cell-free DNA fragment. In some embodiments, this is done by counting (i) how many CpG sites are on the portion of the respective cell-free DNA fragment mapping to the given region (Y_{total_epg_sites}) and, of these, (ii) how many are methylated (X_{cpg_sites_methylated}). It will be appreciated that Y_{total_epg_sites} will be a subset of the CpG sites on the respective cell-free DNA fragment (if the respective cell-free DNA fragment is partially overlapping a region) or all the CpG sites on the fragment (if the cell-free DNA fragment is wholly contained within the given region).

In some embodiments, the methylation configuration (Y_{total_epg_sites}, X_{cpg_sites_methylated}) of the respective cell-free DNA fragment together with the identity of the region the fragment maps to is used to determine whether the cell-free DNA fragment should be designated a circulating-tumor DNA fragment or not. In some embodiments, this is done by using the methylation configuration and the identity of the region it maps to look up the probability that the cell-free DNA fragment is derived from the tumor cells. In some embodiments, such probabilities are available for several different methylation configurations for each region in the plurality of regions. That is, for each methylation configuration in the plurality of methylation configurations for a respective region, a probability between 0 and 1 is provided. Example 1 provides a non-limiting example of the construction of such probabilities for several different methylation configurations for each region in a plurality of regions.

In some embodiments, a cut-off is applied to the probability returned for a respective cell-free DNA fragment based on its methylation configuration and region 110 mapping identity. In some embodiments the cut-off is 0.9. For example, if the look up for a respective cell-free DNA fragment based on its methylation configuration and region 110 returns a probability of 0.89 that the DNA fragment is derived from tumor, and the cutoff is 0.9, the respective cell-free DNA fragment is not labeled a circulating-tumor fragment. As another example, if the look up for a respective cell-free DNA fragment based on its methylation configuration and region 110 returns a probability of 0.91 that the DNA fragment is derived from tumor, and the cutoff is ninety percent, the respective cell-free DNA fragment is labeled a circulating-tumor fragment. In some embodiments, a different threshold is applied for making this cut off decision, such as a value between 0.7 percent and 0.99, a value between −0.75 and 0.90, or a value between 0.80 and 0.95. In some embodiments, the cut off value is 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, or 0.99.

Through the mapping of each respective cell-free DNA fragment in the first plurality of cell-free DNA fragments to respective region in the plurality of regions and the labeling of such mapped fragments based on their mapping and their methylation profiles a corresponding number of circulating tumor DNA fragments 108 mapping to each respective region 112 in the plurality of regions of one or more first reference sequences 110 of the species of the test subject is made using the methylation patterns of the cell-free DNA fragment.

Referring to block 218, in some embodiments, a corresponding expected number of noise fragments 118 is determined in each respective region 112 of the plurality of regions of the one or more first reference sequences 110 of the species of the test subject is made based on a corresponding distribution using an observed sequencing depth from the first sequence reaction and a learned background emission rate for the respective region.

In some embodiments a Bayesian background subtraction process estimates the background emission rate for each region 112. In some embodiments, the background emission is estimated as a distribution, such as a Beta distribution, for each respective region. In some embodiments, for each respective region, a corresponding Beta distribution is learned using a Beta-Binomial model for the respective region trained on the number of unlikely fragments (fragments that satisfied the cutoff value described above) in a plurality of normal plasma samples mapping to the respective regions. For instance, the alpha and beta shape parameters of the Beta distribution are refined until the Beta distribution agrees with the learning set of normal plasma samples. Once the shape parameters of the Beta distribution have been learned using the training set of normal plasma samples for a given region 112, the Beta distribution represents the learned Beta background emission rate for that region.

Referring to block 220, in some embodiments, an excess fragments per million (EFPM) value 120 is determined for the first liquid biopsy sample from the corresponding number of ctDNA fragments in each respective region of the plurality of regions of the one or more first reference sequences observed in the first liquid biopsy sample in excess of the corresponding expected number of noise fragments in the respective region.

In some embodiments, the expected number of noise fragments 118 for a given region (noise) is determined by sampling a Binomial distribution with the observed depth and the learned Beta background emission rate for that region from block 218. Excess fragments for a particular region 112 is then defined as the mean of the differences between the observed fragments and the expected noise:

$$excess {fragments}_{region} = \frac{1}{n} {\sum }_{i}^{n} observed - {noise}_{i}$$

where,
i is an integer index 1, . . . , n, with each i^{th} value representing an i^{th} sampling of the Binomial distribution,
n is the total number of times the Binomial distribution is sampled, and
noiseᵢ, is the expected noise (number of cell-free DNA fragments that are not categorized as from tumor from the i^{th} sampling of the Binomial distribution of block 218 for the given region).

In some embodiments, the Binomial distribution for a particular region in the plurality of regions is sampled five or more times, ten or more times, 15 or more times, 100 or more times, 200 or more times, or 1000 or more times. In some embodiments, rather than a Binomial distributions, a Poisson distribution, Gaussian distribution, Geometric distribution, negative Binomial distribution, hypergeometric distribution, or multinomial distribution is used.

In some embodiments, the EFPM value 120 is the sum of excess fragment measurements (excess fragments_{region}) over all regions 112 in the plurality of regions normalized by the total number of cell-free DNA fragments in the first plurality of cell-free DNA fragments that (i) were not categorized as circulating tumor DNA fragments in block 217 and (ii) that mapped to at least one region in the plurality of regions.

Referring to block 222, in some embodiments, the EFPM value 120 is corrected for the first liquid biopsy sample by an observed CHG methylation level to obtain a corrected EFPM value 122. In such embodiments, the corrected EFPM value 122 is the quantitative estimate for the circulating tumor fraction in the subject. As used herein, CHG methylation is the presence of a methyl (CH₃) group to a cytosine (C) residue in which the cytosine is followed by an adenine, thymine, or cytosine, but not a guanine, then a guanine. CHG methylation is not naturally found within the human genome and is only present in samples as a readout of incomplete reactions during the laboratory process for CpG calling. The process that leads to CHG methylation within the sample will also generate artifactual CpG methylation. However, there is also genuine CpG methylation in the sample, unlike CHG, so the artificially generated CpG signal cannot be observed directly. The CHG methylation rate is a proxy for the artificial CpG methylation signal that can be directly observed and hence a motivation for CHG correction in some embodiments.

In some embodiments to determine the CHG correction value for the EFPM value, the EFPM value for each liquid biological sample from each subject in a plurality of subjects that are known to be free of the cancer condition is determined using the same methods described above. Further, a mean CHG Methylation value is determined for the liquid biological sample of each of these subjects. The mean CHG Methylation value is the average amount of methylation observed at all CHG sites in the genome:

$$\frac{Number of time CHG sites are methylated}{Number of time CHG sites are read}$$

One program for computing mean CHG methylation is MethylDackel, which is open source software tool for extracting and quantifying methylation on reads of DNA sequences. See the Internet at github.com/dpryan79/MethylDackel.

In some embodiments, using the normal plasma EFPM values and the mean CHG methylation for each subject in the plurality of subjects free of the cancer condition, a univariate regression model is fitted to predict the EFPM values from the mean CHG methylation. The predicted value (from the regression) represents the increase in the EFPM value that can be attributed to the observed ChG methylation rate, and thus can be subtracted from the EFPM value in accordance with block 222 to get the corrected EFPM value.

In some embodiments, the corrector itself is a mean and variance centered standard scaling (z-scaling) transformation followed by ridge regression with an intercept that is fit and an alpha value of 1 (constant that multiplies the L2 term, controlling regularization strength), with batch level sample class weighting. The pipeline is then fitted with the single feature mean CHG methylation and the target of EFPM.

Referring to block 224, in some embodiments, a first threshold is applied to the corrected EFPM value 122 to provide a first call for molecular residual disease when the EFPM value 122 satisfies the first threshold or a first call against molecular residual disease when the EFPM value 122 fails to satisfy the first threshold. As noted above, the corrected EFPM value 122 is the quantitative estimate for the circulating tumor fraction in the subject The first threshold is associated with the maximal amount of circulating tumor fraction that can be detected and tolerated when calling a sample negative for the cancer condition. In this instance, it can be referred to as a limit of blank threshold. In some embodiments, the first threshold value will vary from batch to batch of subjects. In some embodiments the first threshold is determined by any approved or standardized clinical and laboratory procedures and guidelines, for example a nonparametric method.

In some embodiments the first threshold for the corrected EFPM value 122 is calculated as a limit of blank (LOB) or a limit of detection (LOD) for circulating tumor fraction in an analysis of biological samples from subjects that are free of the cancer condition measured according to the Clinical and Laboratory Standards Institute guidelines EP17.2 and relevant reports. See, for example, Perry et al., Evaluation of Detection Capability for Clinical Laboratory Measurement Procedures; Approved Guideline—Second Edition Volume 32 Number 8; and Wang et al., 2019, “KRAS Mutant Allele Fraction in Circulating Cell-Free DNA Correlates with Clinical Stage in Pancreatic Cancer Patients,” Front. Oncol (9), 1295, each of which is hereby incorporated by reference.

Because the first threshold is based on evaluation of subjects free of the cancer condition, a precise fixed value for the threshold is dependent on the subjects analyzed to determine the threshold. However, by way of a non-limiting example, consider the case where the threshold is 0.05 and the corrected EFPM value 122 is 0.07. In this example, since the corrected EFPM value 122 of 0.07 exceeds the first threshold value of 0.05, the first threshold is satisfied and a first call for molecular residual disease is made. In another non-limiting example, consider the case where the first threshold is 0.05 and the corrected EFPM value 122 is 0.04. In this example, since the corrected EFPM value 122 of 0.04 is less than the first threshold value of 0.05, the first threshold is not satisfied and a first call against molecular residual disease is made. In more typical instances, EFPM values are on the order of tens or one hundreds.

Referring to block 225, in some embodiments, optionally there is obtained, from a second sequencing reaction, a corresponding sequence of each cell-free DNA fragment 126 in a second plurality of cell-free DNA fragments in a second liquid biopsy sample of the test subject, thereby obtaining a second plurality of sequence reads 127.

In some embodiments, the second sequencing reaction is a duplex sequencing reaction. See Gydush et al., 2022, “Massively-parallel enrichment of minor alleles for mutational testing via low-depth duplex sequencing,” Nat. Biomed Eng, 6(3), pp. 257-266, which is hereby incorporated by reference for disclosure on duplex sequencing reactions.

In some embodiments the second plurality of sequence reads is acquired by any methodology known in the art. For example, next generation sequencing (NGS) techniques such as sequencing-by-synthesis technology (Illumina), pyrosequencing (454 Life Sciences), ion semiconductor technology (Ion Torrent sequencing), single-molecule real-time sequencing (Pacific Biosciences), sequencing by ligation (SOLID sequencing), or nanopore sequencing (Oxford Nanopore Technologies) is performed. In some embodiments sequencing is performed by next generation sequencer (e.g., an Illumina HiSeq 4000, Illumina NovaSeq 6000, etc.). In some such embodiments this sequencing is a paired-end sequencing is performed. In some embodiments, massively parallel sequencing is performed using sequencing-by-synthesis with reversible dye terminators. In some embodiments, sequencing is performed using next generation sequencing technologies, such as short-read technologies. In other embodiments, long-read sequencing or another sequencing method known in the art is used.

In some embodiments the second sequencing reaction is NGS that produces hundreds of thousands, millions, or hundreds of millions of sequence reads from the second plurality of cell-free DNA fragments in the biological sample (e.g., after fragmentation of the cell-free DNA). Accordingly, in some embodiments, the plurality of sequence reads obtained by NGS of cell-free DNA fragments are DNA sequence reads. In some embodiments, the sequence reads in the second plurality of sequence reads have an average length of at least fifty nucleotides. In other embodiments, the sequence reads in the second plurality of sequence reads have an average length of at least 50, 60, 70, 80, 90, 100, 150, 200, 250, 300, or more nucleotides.

In some embodiments, the second sequencing reaction is performed after enriching for nucleic acids (e.g., cfDNA, gDNA, and/or RNA) encompassing a plurality of predetermined target sequences, e.g., human genes and/or non-coding sequences associated with cancer in accordance with block 230 discussed in further detail below. Advantageously, sequencing a nucleic acid sample that has been enriched for target cell-free DNA fragments, rather than all cell-free DNA fragments isolated from the second biological sample, significantly reduces the average time and cost of the second sequencing reaction. Accordingly, in some embodiments, the methods described herein include obtaining a second plurality of sequence reads of cell-free DNA fragments that have been hybridized to a probe set for hybrid-capture enrichment (e.g., of one or more genes listed in Table 1 below).

Referring to block 226, in some embodiments, the first liquid biopsy sample and the second liquid biopsy sample are the same liquid biopsy sample. For instance, a common liquid biopsy sample is obtained from the subject and then split into two aliquots, one for the first sequencing reaction and the other for the second sequencing reaction.

Referring to block 228, in some embodiments, the first liquid biopsy sample and the second liquid biopsy sample are different liquid biopsy samples.

Referring to block 230, in some embodiments, the second sequencing reaction is a panel-based sequencing reaction of a plurality of loci. In some such embodiments a plurality of nucleic acid probes (e.g., a probe set) is used to enrich one or more target sequences in the second liquid biopsy sample for the plurality of loci. In some embodiments, the probe set includes probes targeting one or more gene loci, e.g., exon or intron loci within the plurality of loci. In some embodiments, the plurality of loci enriched by the probe set includes one or more loci not encoding a protein, e.g., regulatory loci, miRNA loci, and other non-coding loci, e.g., that have been found to be associated with cancer. In some embodiments, the plurality of loci includes at least 25, 50, 100, 150, 200, 250, 300, 350, 400, 500, 750, 1000, 2500, 5000, or more human genomic loci.

In some embodiments, the plurality of loci includes one more of the genes listed in Table 1. In some embodiments, the plurality of loci includes at least 5 of the genes listed in Table 1. In some embodiments, the plurality of loci includes at least 10 of the genes listed in Table 1. In some embodiments, the plurality of loci includes at least 25 of the genes listed in Table 1. In some embodiments, the plurality of loci includes at least 50 of the genes listed in Table 1. In some embodiments, the plurality of loci includes at least 75 of the genes listed in Table 1. In some embodiments, the plurality of loci includes at least 100 of the genes listed in Table 1. In some embodiments the plurality of loci consists of or comprises all of the genes listed in Table 1.

TABLE 1
An example panel of 105 genes.
Table 1: Liquid Biopsy Gene Panel

| | | | | | | |
|---|---|---|---|---|---|---|
| ALK | B2M | ERRFI1 | IDH2 | MSH6 | PIK3R1 | SPOP |
| FGFR2 | BAP1 | ESR1 | JAK1 | MTOR | PMS2 | STK11 |
| FGFR3 | BRCA1 | EZH2 | JAK2 | MYCN | PTCH1 | TERT |
| NTRK1 | BRCA2 | FBXW7 | JAK3 | NF1 | PTEN | TP53 |
| RET | BTK | FGFR1 | KDR | NF2 | PTPN11 | TSC1 |
| ROS1 | CCND1 | FGFR4 | KEAP1 | NFE2L2 | RAD51C | TSC2 |
| BRAF | CCND2 | FLT3 | KIT | NOTCH1 | RAF1 | UGT1A1 |
| AKT1 | CCND3 | FOXL2 | KRAS | NPM1 | RB1 | VHL |
| AKT2 | CDH1 | GATA3 | MAP2K1 | NRAS | RHEB | CCNE1 |
| APC | CDK4 | GNA11 | MAP2K2 | PALB2 | RHOA | CD274 |
| AR | CDK6 | GNAQ | MAPK1 | PBRM1 | RIT1 | EGFR |
| ARAF | CDKN2A | GNAS | MLH1 | PDCD1LG2 | RNF43 | ERBB2 |
| ARID1A | CTNNB1 | HNF1A | MPL | PDGFRA | SDHA | MET |
| ATM | DDR2 | HRAS | MSH2 | PDGFRB | SMAD4 | MYC |
| ATR | DPYD | IDH1 | MSH3 | PIK3CA | SMO | KMT2A |

Generally, probes for enrichment of nucleic acids (e.g., cfDNA obtained from a liquid biopsy sample) include DNA, RNA, or a modified nucleic acid structure with a base sequence that is complementary to a locus of interest. For instance, a probe designed to hybridize to a locus in a cell-free DNA fragment can contain a sequence that is complementary to either strand, because the cell-free DNA fragments are double stranded. In some embodiments, each probe in the plurality of probes includes a nucleic acid sequence that is identical or complementary to at least 10, at least 11, at least 12, at least 13, at least 14, or at least 15 consecutive bases of a locus in the plurality of loci. In some embodiments, each probe in the plurality of probes includes a nucleic acid sequence that is identical or complementary to at least 20, 25, 30, 40, 50, 75, 100, 150, 200, or more consecutive bases of a locus in the plurality of loci.

In some embodiments, the plurality of loci is a whole-exome panel comprising the exomes of a biological sample. In some embodiments, the plurality of loci is a whole-genome panel that comprises the genome of a specimen.

In some embodiments, the probes for the second sequencing region in accordance with block 230 include additional nucleic acid sequences that do not share any homology to the plurality of loci. For example, in some embodiments, the probes also include nucleic acid sequences containing an identifier sequence, e.g., a unique molecular identifier (UMI), e.g., that is unique to a particular sample or subject. Similarly, in some embodiments, the probes also include primer nucleic acid sequences useful for amplifying the nucleic acid molecule of interest, e.g., using PCR. In some embodiments, the probes also include a capture sequence designed to hybridize to an anti-capture sequence for recovering the nucleic acid molecule of interest from the sample.

Likewise, in some embodiments, the probes each include a non-nucleic acid affinity moiety covalently attached to a nucleic acid molecule that is complementary to a locus in the plurality of loci, for recovering a cell-free DNA fragment. Non-limited examples of non-nucleic acid affinity moieties include biotin, digoxigenin, and dinitrophenol. In some embodiments, the probe is attached to a solid-state surface or particle, e.g., a dip-stick or magnetic bead, for recovering the nucleic acid of interest. In some embodiments, the methods described herein include amplifying the nucleic acids that bound to the probe set prior to further analysis, e.g., sequencing. Methods for amplifying nucleic acids, e.g., by PCR, are well known in the art.

In some embodiments, panel-targeting sequencing is performed to an average on-target depth of at least 500×, at least 750×, at least 1000×, at least 2500×, at least 500×, at least 10,000×, or greater depth.

Referring to block 232, in some embodiments, the plurality of loci is sequenced at an average sequence depth of at least 250× by the second sequencing reaction. Referring to block 234, in some embodiments, the plurality of loci is sequenced at an average sequence depth of at least 1000× by the second sequencing reaction. In some embodiments, the plurality of loci is sequenced at an average sequence depth of at least 500×, at least 750×, at least 1000×, at least 2500×, at least 500×, at least 10,000×, or greater.

Referring to block 236, in some embodiments, the second plurality of sequence reads comprises at least 50,000 sequence reads or at least 250,000 sequence reads. In some embodiments the second plurality of sequence reads comprises at least 1000, at least 2000, at least 3000, at least 4000, at least 5000, at least 6000, at least 7000, at least 8000, at least 9000, at least 10,000, at least 50,000, at least 100,000, at least 500,000, at least 1 million, at least 2 million, at least 3 million, at least 4 million, at least 5 million, at least 6 million, at least 7 million, at least 8 million, at least 9 million, or more sequence reads. In some embodiments, the second plurality of sequence reads consists of between 50,000 sequence reads and 10 million sequence reads. In some embodiments, the second plurality of sequence reads consists of between 100,000 sequence reads and 8 million sequence reads. In some embodiments, the second plurality of sequence reads consists of between 200,000 sequence reads and 6 million sequence reads.

In some embodiments each respective sequence read in the second plurality of sequence reads is mapped to one or more second reference sequences (e.g., a reference human genome) by identifying a sequence in a region of the one or more second reference sequences that best matches the sequence of nucleotides in the respective sequence read. In some embodiments, this mapping uses methods known in the art to determine alignment position information. The alignment position information may indicate a beginning position and an end position of a region in the one or more second reference sequences that corresponds to a beginning nucleotide base and end nucleotide base of the respective sequence read. Alignment position information may also include the sequence read length, which can be determined from the beginning position and end position. A region in the one or more reference sequences may be associated with a gene or a segment of a gene. Any of a variety of alignment tools can be used to optimize the mapped alignment.

For instance, local sequence alignment algorithms compare subsequences of different lengths in the sequence read to subsequences in the one or more reference sequences to create the best alignment for each portion of the query sequence. In contrast, global sequence alignment algorithms align the entirety of the sequence read.

In some embodiments, the sequence read mapping process starts by building an index of either the one or more reference sequences or the second plurality of sequences reads, which is then used to retrieve the set of positions in the one or more reference sequences where a sequence read is more likely to align. Once this subset of possible mapping locations has been identified, alignment is performed in these candidate regions with slower and more sensitive algorithms. In some embodiments, the mapping methodology makes use of a hash table or a Burrows-Wheeler transform (BWT). See, for example, Li and Homer, 2010, “A survey of sequence alignment algorithms for next-generation sequencing,” Brief Bioinformatics 11, pp. 473-483, which is hereby incorporated by reference.

Other software programs designed to align sequence reads to reference sequences include, but are not limited to, Novoalign (Novocraft, Inc.), Bowtie, Burrows Wheeler Aligner (BWA), and/or programs that use a Smith-Waterman algorithm. Example reference sequences includes genomes such as, for example, hg19, GRCh38, hg38, GRCh37, and/or other reference genomes developed by the Genome Reference Consortium.

In some embodiments, each respective sequence read in the second plurality of sequence reads is in FASTQ file format and is aligned to a location in the one or more second reference sequences (e.g., a human genome) having a sequence that best matches the sequence of nucleotides in the respective sequence read. There are many software programs designed to map sequence reads, for example, Novoalign (Novocraft, Inc.), Bowtie, Burrows Wheeler Aligner (BWA), programs that use a Smith-Waterman algorithm, etc. Alignment may be directed using a reference genome (for example, hg19, GRCh38, hg38, GRCh37, other reference genomes developed by the Genome Reference Consortium, etc.) by comparing the nucleotide sequences in each read with portions of the nucleotide sequence in the reference genome to determine the portion of the reference genome sequence that is most likely to correspond to the sequence in the read. In some embodiments, one or more SAM files are generated for the alignment, which store the locations of the start and end of each read according to coordinates in the reference genome and the coverage (number of reads) for each nucleotide in the reference genome. The SAM files may be converted to BAM files. In some embodiments, the BAM files are sorted and duplicate reads are marked for deletion, resulting in de-duplicated BAM files.

In some embodiments, adapter-trimmed FASTQ files are aligned to the 19th edition of the human reference genome build (HG19) using Burrows-Wheeler Aligner (BWA) [PMC2705234]. Following alignment, the sequence reads are grouped by alignment position and UMI family and collapsed into consensus sequences, for example, using fgbio tools (http://fulcrumgenomics.github.io/fgbio/). Bases with insufficient quality or significant disagreement among family members (for example, when it is uncertain whether the base is an adenine, cytosine, guanine, etc.) may be replaced by N's to represent a wildcard nucleotide type. PHRED scores are then scaled based on initial base calling estimates combined across all family members. Following single-strand consensus generation, duplex consensus sequences are generated by comparing the forward and reverse oriented PCR products with mirrored UMI sequences. In various embodiments, a consensus can be generated across read pairs. Otherwise, single-strand consensus calls will be used. Following consensus calling, in some embodiments, filtering is performed to remove low-quality consensus fragments. In some embodiments, the consensus fragments are then re-aligned to the human reference genome using BWA. In some embodiments, a BAM output file is generated after the re-alignment, then sorted by alignment position, and indexed.

In some embodiments, the sequencing data is normalized, e.g., to account for pull-down, amplification, and/or sequencing bias (e.g., mappability, GC bias etc.). See, for example, Schwartz et al., 2011, PLOS ONE 6(1):e16685; and Benjamini and Speed, 2012, Nucleic Acids Research 40(10):e72, each of which is hereby incorporated by reference, for disclosure on types of biases that can be removed from sequencing data.

Referring to block 238 of FIG. 2D, in some embodiments, the one or more first reference sequences 110 is a human reference genome, the one or more second reference sequences is also the human reference genome, the plurality of regions (for the first sequencing reaction) comprises 1000 or more regions cumulatively mapping to between four megabases and ten megabases of the human reference genome, the second sequencing reaction is a panel-based sequencing reaction of a plurality of loci, and the plurality of loci comprises 50 or more loci cumulatively mapping to between 0.1 megabase and 1 megabase of the human reference genome. FIG. 3 illustrates such an embodiment. In FIG. 3 the plurality of regions (for the first sequencing reaction 302) comprises thousands of differentially methylated loci cumulatively mapping to 5.9 megabases of the human reference genome, the second sequencing reaction 304 is a panel-based sequencing reaction of over 100 genes cumulatively mapping to 0.3 megabase of the human reference genome.

Referring to block 240, in some embodiments, the second plurality of sequence reads is used to identify each respective candidate somatic variant 130 in a candidate somatic variant set 128. Each candidate somatic variant 128 in the set of candidate somatic variants is a single nucleic acid variant (SNV). In some embodiments, identification of block 240 results in the identification of a candidate somatic variant set that comprises 10 or more, 15 or more, 20 or more, 25 or more, 30 or more, 35 or more, 40 or more, 45 or more, 50 or more, 100 or more, 200 or more, 300 or more, 400 or more, 500 or more, 1000 or more, 2000 or more, 3000 or more, 4000 or more, 5000 or more or 10,000 or more candidate somatic variants. In some embodiments, each candidate somatic variant maps to a different portion of the genome of a species.

Referring to block 242, in some embodiments, each respective candidate somatic variant 130 in the set of candidate somatic variants 128 is identified by applying a variant caller to the second plurality of sequence reads 127 with a restriction that the variant caller determines that each respective candidate somatic variant in the set of candidate somatic variants has a variant allele frequency of at least 0.1 in the second plurality of sequence reads and that at least one cell-free DNA fragment in the second plurality of cell-free DNA fragments exhibits the respective candidate somatic variant. As used herein, the term “variant allele frequency,” “VAF,” “allelic fraction,” or “AF” refer to the number of times a variant or mutant allele was observed in the second plurality of sequence reads (e.g., a number of reads supporting a variant allele) divided by the total number of times the position the variant or mutant allele occupies was sequenced (e.g., a total number of reads covering a candidate locus).

In some embodiments, candidate somatic variant detection is accomplished using VarDict (available on the internet at github.com/AstraZeneca-NGS/VarDictJava). Both SNVs and INDELs are called and then sorted, deduplicated, normalized and annotated. In some embodiments, the annotation uses SnpEff to add transcript information, 1000 genomes minor allele frequencies, COSMIC reference names and counts, ExAC allele frequencies, and Kaviar population allele frequencies. The annotated variants are then classified as germline, somatic, or uncertain using a Bayesian model based on prior expectations informed by databases of germline and cancer variants. In some embodiments, uncertain variants are treated as somatic for filtering and reporting purposes.

Referring to block 243 of FIG. 2E, in some embodiments, the set of candidate somatic variants 128 is filtered by a procedure.

In some embodiments, the procedure comprises removing from the set of candidate allele variants each candidate allele variant in the set of candidate allele variants that has a variant allele fraction exceeding an upper threshold fraction in the second plurality of sequence reads. In some embodiments this upper threshold fraction is 0.40 meaning that no more than forty percent of the cell-free DNA fragments identified using the second plurality of sequence reads mapping to a given loci can have the same alternate (non-wild type) allele. In some embodiments this upper threshold fraction is a real value between 0.20 and 0.80 (e.g., 0.20, 0.21, 0.22, 0.23, 0.24, 0.25, . . . , 0.70, 0.71, 0.72, 0.73, 0.74, 0.75, 0.76, 0.77, 0.78, 0.79, or 0.80).

Referring to block 244, in some embodiments, the procedure comprises removing from the set of candidate somatic variants 128 each respective candidate somatic variant 130 that is present in a repository of known germline variants.

In some embodiments, variants are present in a repository of known germline variants when they are represented in the population above a threshold level, e.g., as determined using a population database such as ExAC or gnomAD. For instance, in some embodiments, variants that are represented in at least 1% of the alleles in a population are annotated as germline in the repository. In other embodiments, variants that are represented in at least 2%, at least 3%, at least 4%, at least 5%, at least 7.5%, at least 10%, or more of the alleles in a population are annotated as germline in the repository. In some embodiments, sequencing data from a matched sample from the subject, e.g., a normal tissue sample, is used to annotate variants identified in the second liquid biopsy sample from the subject. That is, variants that are present in both the second liquid biopsy sample and a normal sample represent those variants that were in the germline and can be annotated in the repository as germline variants.

In some embodiments germline variants are identified using a program such as HAPLOTYPE CALLER (e.g., version 3.1-1) [DePristo et al., 2011, “A framework for variation discovery and genotyping using next-generation DNA sequencing Data,” Nat Genet. 43:491-498], samtools (e.g., version 1.2) (Li, 2011, “A statistical framework for SNP calling, mutation discovery, association mapping and population genetical parameter estimation from sequencing data,” Bioinforma Oxf Engl. 27, pp. 2987-2993) or freebayes (e.g., v0.9.21) [Garrison et al., 2015, “Haplotype-based variant detection from short-read sequencing.,” ArXiv Prepr ArXiv12073907].

Referring to block 246 of FIG. 2E, in some embodiments, the procedure comprises removing from the set of candidate somatic variants 128 each respective candidate somatic variant 130 in the set of candidate somatic variants that maps to a variant interval in a plurality of variant intervals, where each respective variant interval in the plurality of variant intervals is identified as having a pre-test odds of a positive variant call that is less than a pre-test odds threshold value based upon a prevalence of a corresponding one or more training variants, which are each above limit of detection and map to the respective variant interval, in a plurality of tumor-normal matched samples obtained from a first cohort of training subjects having the cancer condition with the proviso that no variant in a second cohort of healthy samples maps to the respective variant interval.

Example 3 illustrates identifying the plurality of variant intervals that each have a pre-test odds of a positive variant call that is greater than the pre-test odds threshold value based upon a prevalence of a corresponding one or more training variants, which are each above limit of detection and map to the respective variant interval, in a plurality of tumor-normal matched colorectal samples obtained from a first cohort of training subjects having the cancer condition with the proviso that no variant in a second cohort of healthy samples maps to the respective variant interval. Thus, in accordance with block 246, in some embodiments, any candidate variant allele that does not map to a variant interval identified in Example 3 as having a pre-test odds greater than pre-test odds threshold value is removed from the set of candidate variant alleles. In other words, for colorectal cancer, Example 3 identifies the regions (variant intervals) of the human genome where there is high confidence that alleles that are associated with the colorectal cancer state are to be found. Thus, if a candidate variant allele in the set of candidate variant allele does not map to one of these regions (variant intervals), the candidate variant allele is removed from the set of candidate variant alleles in accordance with block 246.

As detailed in Example 3, a variant interval can encompass more than one variant if such variants are adjacent to each other in the human genome. Variant intervals as long as 7 residues were found in Example 3. Thus, in instances where a respective candidate variant allele is a SNP, the mapping that occurs in block 246 only has to find that this SNP maps to any position in the variant interval.

In some embodiments each variant interval in the plurality of variant intervals consists of between 1 base and 25 bases. In some embodiments each variant interval in the plurality of variant intervals consists of between 1 base and 20 bases. In some embodiments each variant interval in the plurality of variant intervals consists of between 1 base and 15 bases. In some embodiments each variant interval in the plurality of variant intervals consists of between 1 base and 10 bases. In some embodiments each variant interval in the plurality of variant intervals consists of between 1 base and 10 bases.

In some embodiments, the pre-test odds threshold value is 0.001. In some embodiments, the pre-test odds threshold value is a value between 0.01 and 0.0001. In some embodiments, the pre-test odds threshold value is 0.0005, 0.002, 0.003, 0.004, or 0.005.

In some embodiments, the plurality of variant intervals comprises between 10 and 5000 variant intervals, between 20 and 4500 variant intervals, between 30 and 4000 variant intervals, between 40 and 3500 variant intervals, between 50 and 3000 variant intervals, or between 60 and 2500 variant intervals.

In some embodiments, the plurality of tumor-matched samples for the cancer condition comprises 10 or more, 20 or more, 30 or more, 40 or more, 50 or more, 60 or more, 70 or more, 80 or more, 90 or more, 100 or more, 200 or more, 300 or more, 400 or ore, 500 or more, 600 or more, 700 or more, 1000 or more, 1500 or more, or 2000 or more tumor-matched samples. In some embodiments the plurality of variant intervals is obtained from variants identified in the tumor-matched samples using a tumor-normal matched, targeted DNA-seq panel that detects single nucleotide variants, insertions and/or deletions, and copy number variants in 598-648 genes, as well as chromosomal rearrangements in 22 genes with high sensitivity and specificity at 500× coverage, for example, the Tempus xT assay (Tempus, Chicago, Illinois).

In some embodiments, the second cohort of healthy samples comprises 10 or more, 20 or more, 30 or more, 40 or more, 50 or more, 60 or more, 70 or more, 80 or more, 90 or more, 100 or more, 200 or more, 300 or more, 400 or ore, 500 or more, 600 or more, 700 or more, 1000 or more, 1500 or more, or 2000 or more healthy samples. In some embodiments variants in the healthy samples that are used to disqualify variant intervals in the plurality of variant intervals are identified using a non-invasive, liquid biopsy panel of 105 (v2) or 523 (v3) genes focused on detection of oncogenic and resistance mutations, including SNVs and INDELs, CNVs, genomic rearrangements, and MSI status. See Finkle J D, Boulos H, Driessen T M, et al. Validation of a liquid biopsy assay with molecular and clinical profiling of circulating tumor DNA. NPJ Precis Oncol. 2021; 5(1):63, for example, the Tempus xF assay (Tempus, Chicago, Illinois). In some embodiments in accordance with block 246, a variant is considered to be in the second cohort of healthy samples when more than 1 cell-free DNA fragment possessing the variant is detected in the cohort of healthy samples (either after filtering using the criteria of blocks 244, 250, 252, 256 or not filtering at all). Example 3 illustrates such an embodiment. In some embodiments in accordance with block 246, a variant is considered to be in the second cohort of healthy samples when a single cell-free DNA fragment possessing the variant is detected in the cohort of healthy samples (either after filtering using the criteria of blocks 244, 250, 252, 256 or not filtering at all). That is, if any healthy sample in the cohort of healthy samples possess the variant. In some embodiments in accordance with block 246, a respective variant is considered to be in the second cohort of healthy samples when more than 2, 3, 4, or 5 cell-free DNA fragments possessing the respective variant are detected in the cohort of healthy samples (either after filtering using the criteria of blocks 244, 250, 252, 256 or not filtering at all).

Referring to block 250, in some embodiments, the procedure includes removing from the set of candidate somatic variants 130 each respective candidate somatic variant 128 in the set of candidate somatic variants that is identified as an artifactual variant. In some embodiments the respective candidate variant is identified as an artifactual variant by being listed in a first data structure. The variants in this first data structure are frequently identified as artifactual variants in high-throughput sequencing studies. Artifactual variants can be generated as a result of copying damaged DNA bases in preparation for or during the second sequencing reaction of block 225. Such variants are present on only one of the two DNA strands and are scored by conventional next generation sequencing (NGS) and single strand consensus sequence (SSCS) but not by duplex consensus sequence (DCS) analysis of duplex sequencing.

The Pan-cancer Analysis of Whole Genomes (PCAWG) consortium has generated 65 single-base substitution (SBS) mutational signatures from over 4600 whole cancer genomes and 19,000 cancer exomes. See Alexandrov et al., 2018, “The repertoire of mutational signatures in human cancer,” bioRxiv 322859, which is hereby incorporated by reference. These signatures have been incorporated as version 3 into the v89 release of Catalog of Somatic Mutations in Cancer (COSMIC). See Forbes et al., 2017, “COSMIC: somatic cancer genetics at high-resolution,” Nucleic Acids Res 45, pp. D777-83, which is hereby incorporated by reference. Such data has been used to develop software that can detect artifactual variants, such as FIREVAT. See Kim et al., 2019, “FIREVAT: finding reliable variants without artifacts in human cancer samples using etiologically relevant mutational signatures,” Genome Medicine 11(81), which is hereby incorporated by reference.

In some embodiments, the procedure comprises removing from the set of candidate somatic variants 130 each respective candidate somatic variant 128 that is identified as being observed in a cohort of healthy subjects. In some embodiments the variant is identified as being observed in a cohort of healthy subjects by being listed in a second data structure. The variants in this second data structure are frequently found in a cohort of healthy subjects. In some embodiments, the cohort of healthy subjects of block 250 comprises 10 or more, 20 or more, 30 or more, 40 or more, 50 or more, 60 or more, 70 or more, 80 or more, 90 or more, 100 or more, 200 or more, 300 or more, 400 or ore, 500 or more, 600 or more, 700 or more, 1000 or more, 1500 or more, or 2000 or more healthy subjects. In some embodiments a variant is identified as being observed in the cohort of healthy subjects by using the Tempus xF assay (Tempus, Chicago, Illinois). In some embodiments in accordance with block 250, a variant is considered to be in the cohort of healthy subjects when more than 1 cell-free DNA fragment possessing the variant is detected in the cohort of healthy subjects (either after filtering using the criteria of blocks 244, 250, 252, 256 or not filtering at all). In some embodiments in accordance with block 250, a variant is considered to be in the cohort of healthy subjects when a single cell-free DNA fragment possessing the variant is detected in the cohort of healthy subjects (either after filtering using the criteria of blocks 244, 250, 252, 256 or not filtering at all). In some embodiments in accordance with block 250, a variant is considered to be in the cohort of healthy subjects when more than 2, 3, 4, or 5 cell-free DNA fragments possessing the variant are detected in the cohort of healthy subjects (either after filtering using the criteria of blocks 244, 250, 252, 256 or not filtering at all).

Referring to block 252, in some embodiments, the procedure comprising removing from the set of candidate somatic variants each respective candidate somatic variant in the set of candidate somatic variants in which the second sequencing reaction produced a coverage depth of less than a threshold amount for the respective locus in one or more second reference sequences of the species of the subject that the candidate somatic variant maps to.

In some embodiments a minimum of 250× sequencing depth must be observed for the locus a candidate somatic variant in order to retain the candidate somatic variant. In some embodiments, a minimum 50×, 100×, 150×, 200×, 300×, 350×, 400×, 450×, 500×, 1000×, or 2000× sequencing depth must be observed for the locus a candidate somatic variant in the second sequencing reaction maps to in order to retain the candidate somatic variant. Such sequencing depths in accordance with block 252 refer to the actual locus in the one or more second reference sequences that the candidate somatic variant maps to, as opposed to the overall depth of the second sequencing reaction.

In some embodiments, the threshold sequencing depth is a duplex sequencing depth in accordance with the definition given for duplex sequencing depth in the definitions section above. In some such embodiments, the locus a candidate somatic variant maps to must have at least a 50×, 100×, 150×, 200×, 300×, 350×, 400×, 450×, 500×, 1000×, or 2000× duplex sequencing depth in the second sequencing reaction in order to be retained as a candidate somatic variant.

In considering the threshold sequencing depth of block 252, not all of the cell-free DNA fragments mapping to the locus that a given candidate somatic variant maps to need to have the mutant allele. For instance, in the case where the coverage depth threshold amount is 50×, 49 cell-free DNA fragments having the wild-type allele and one cell-free DNA fragment mapping to the locus that the given candidate somatic variant maps to would be sufficient to meet the coverage requirements.

In some embodiments, there is a requirement that, in order to retain a given candidate somatic variant in the set of given candidate somatic variants, both strands of at least one cell-free nucleic acid fragment bearing the mutant (alt) allele be identified from (supported by) the second sequencing reaction. Example 5 provides one example of how this duplex consensus read support can affect specificity and sensitivity. In some embodiments, there is a requirement that, in order to retain a given candidate somatic variant in the set of given candidate somatic variants, both strands of at least 2, 3, 4, or 5 cell-free nucleic acid fragments bearing the mutant (alt) allele of the given candidate somatic variant be identified from (supported by) one or more sequence reads of the second plurality of sequence reads of the second sequencing reaction.

Referring to block 254, in some embodiments, the procedure comprises removing from the set of candidate variants 128 each respective candidate somatic variant 130 in the set of candidate somatic variants that (a) maps to a repeat region in the one or more second reference sequences of the species and (b) is not annotated as a known somatic mutation in a database of known somatic mutations for the species of the subject. Repeat regions are repeating sequences of two or more base pairs that are adjacent to one another and are abundant throughout genomes, such as the human genome. See, for example, Madsen et al., 2008, “Short tandem repeats in human exons: a target for disease mutations,” BMC genomics, 9, p. 410, which is hereby incorporated by reference. Databases of known somatic mutations for humans include, but are not limited to, Clin Var and COSMIC. See, Landrum et al., 2020, “ClinVar: improvements to accessing data,” Nucleic Acids Res. 48 (D1), pp. D835-D844; and Tate et al., “COSMIC: the Catalogue of Somatic Mutations in Cancer,” Nucleic Acids Research 47 D1, pp. D941-D947, each of which is hereby incorporated by reference. Thus, in some embodiments, if a candidate somatic variant maps to a repeat region and is not in ClinVar it is removed from the set of candidate somatic variants. In some embodiments, if a candidate somatic variant maps to a repeat region and is not in COSMIC it is removed from the set of candidate somatic variants. In some embodiments, if a candidate somatic variant maps to a repeat region and is not in COSMIC or in Clin Var it is removed from the set of candidate somatic variants. Clin Var and COSMIC are nonlimiting examples of databases of known somatic mutations and other databases, or any combination of such databases may be used for condition (b) of block 254.

Referring to block 256, in some embodiments, the procedure comprises removing from the set of candidate somatic variants 128 each respective candidate somatic variant 130 in the set of candidate somatic variants that maps to a region of clonal hematopoiesis of indeterminate potential (CHIP). Such embodiments are employed to mitigate the risk that a CHIP variant is passed as a candidate somatic variant.

CHIP describes an expansion of hematopoietic stem cells that harbor somatic mutations without an underlying malignancy. CHIP has been identified through genomic profiling of peripheral blood from healthy individuals. See, Busque et al., 2012, “Recurrent somatic TET2 mutations in normal elderly individuals with clonal hematopoiesis,” Nat Genet. 44(11), pp. 1179-1181, which is hereby incorporated by reference. Its incidence increases with age and has been detected in peripheral blood of patients with solid tumors. See, Xie et al . . . , 2014, “Age-related mutations associated with clonal hematopoietic expansion and malignancies,” Nat Med. 20(12), pp, 1472-1478, which is hereby incorporated by reference. Hematopoietic cells permeate all tissues and are present in solid tumor specimens. See Severson et al., 2018, “Detection of clonal hematopoiesis of indeterminate potential in clinical sequencing of solid tumor specimens,” Blood 131(22), pp. 2501-2502, which is hereby incorporated by reference. The application of comprehensive genomic profiling (CGP) to tumor samples provides an unbiased view of heterogeneous cancer cells and admixed nontumor populations.

In one approach to mitigate the risk that a CHIP variant is passed as a candidate somatic variant, an upper bound on how frequently it is expected that the CHIP variant would be passed as a candidate somatic variant is determined by using historical CHIP variant prevalence from comprehensive genomic profiling of tumor samples, such as Tempus xT (Beaubier et al., 2019, “Clinical validation of the Tempus xT next-generation targeted oncology sequencing assay,” Oncotarget 10, pp. 2384-2396, which is hereby incorporated by reference), and calculating the frequency it would, in fact, alter an MRD call on the basis that the CHIP variant was given high confidence in block 246 and the logic for calling MRD ultimately used in block 262 and/or block 263 characterizing an MRD+/− call. In some embodiments such historical presence is matched to an age bracket of the subject. For instance, in some embodiments, only that historical CHIP variant prevalence from comprehensive genomic profiling of tumor samples matched to the subject's age bracket is considered.

In some embodiments samples in a test set that have been tested on both tumor (e.g., xT) and liquid biopsy (e.g., xF), the tumor data can be used to label likely CHIP variants in the liquid biopsy sample to test how likely CHIP variants are passed as a candidate variant for MRD. The xT and xF assays are described in Beaubier et al., 2019, “Clinical validation of the tempus xT next-generation targeted oncology sequencing assay,” Oncotarget 10, pp. 2384-2396, which is hereby incorporated by reference. In some embodiments a CHIP likelihood ratio is calculated using historical xF data based on CHIP variant prevalence in xT. In some embodiments, an empirically determined threshold of 20% was found to 1) filter SNPs in xF for MRD that had a higher likelihood of being classified as CHIP in xT, and 2) improved specificity in MRD calling. In some embodiments a threshold of between 0.10 and 0.45 is used. In some embodiments, the threshold is 0.10, 01.15, 0.20, 0.25, 0.30, 0.35 or 0.40.

In some embodiments, in accordance with block 258, the procedure comprises removing from the set of candidate somatic variants 128 each respective candidate somatic variant 130 in the set of candidate somatic variants that maps to ASXL1, BCOR, BCORL1, CBL, CREBBP, CUX1, DNMT3A, GNB1, JAK2, PPMID, PRPF8, SETDB1, SF3B1, SRSF2, TET2, U2AF1 or any combination thereof. In some embodiments any subset of these genes is used for the CHIP filter. In some embodiments this set of genes, or any subset thereof, is used for the CHIP filter when the cancer condition is a BRCA-associated cancer. See Marshall et al., “Germline mutations and the presence of clonal hematopoiesis of indeterminate potential (CHIP) in 20,963 patients with BRCA-associated cancers.,” DOI: 10.1200/JCO.2023.41.16_suppl.10522 Journal of Clinical Oncology 41, no. 16_suppl (Jun. 1, 2023) 10522-10522, which is hereby incorporated by reference.

In some embodiments, the procedure comprises removing from the set of candidate somatic variants 128 each respective candidate somatic variant 130 in the set of candidate somatic variants that maps to TET2, DNMT3A, ASXL1, or SF3B1. In some embodiments any subset set of these genes is used for the CHIP filter of block 256. In some embodiments this set of genes, or any subset thereof, is used for the CHIP filter when the cancer condition is a BRCA-associated cancer.

In some embodiments, in accordance with block 260, the procedure comprises removing from the set of candidate somatic variants 128 each respective candidate somatic variant 130 in the set of candidate somatic variants that maps to TET2, DNMT3A, ASXL1, SF3B1, CBL, U2AF1, IDH2,2,3, MYD88, 13, EP300, CDKN2C, HNF1A. In some embodiments any subset set of these genes is used for the CHIP filter of block 256.

In some embodiments, the procedure comprises removing from the set of candidate somatic variants 128 each respective candidate somatic variant 130 in the set of candidate somatic variants that maps to TET2, DNMT3A, ASXL1, SF3B1, CBL, U2AF1, IDH2,2,3, MYD88, 13, EP300, CDKN2C, or HNF1A when the subject is 70 years of age or older. Such genes are known CHIP genes in subjects of this age. See, Severson, 2018, “Detection of clonal hematopoiesis of indeterminate potential in clinical sequencing of solid tumor specimens,” Blood 131(22), pp. 2501-2505, which is hereby incorporated by reference.

In some embodiments, CHIP variants are filtered by using a machine learning model trained to identify if a variant is likely to be a CHIP variant. In some embodiments the model is a random forest model such as the model described in Example 3 of U.S. Provisional Patent Application No. 63/574,751, entitled “METHODS AND SYSTEMS FOR FILTERING CLONAL HEMATOPOIESIS VARIANTS IN A LIQUID BIOPSY ASSAY,” filed Apr. 4, 2024, which is hereby incorporated by reference, hereinafter “the '751 Application.” In some embodiments the ensemble model is XGBoost or LightGBM. See e.g., Chen et al. 2016 KDD '16: Proc 22nd ACM SIGKDD Int Conf Knowledge Disc. Data Mining, 785-794, and Wang et al. 2017 ICCBB: Proc 2017 Int Conf Comp Biol and Bioinform, 7-11. In some embodiments the ensemble model is a random forest. In some embodiments the ensemble model is a bagging (bootstrap aggregating) model such as a random forest model or a bagged decision tree model. In some embodiments the ensemble model is boosting model such as XGBoost, a gradient boosting machine, AdaBoost (Adaptive Boosting), LightGBM, or CatBoost. In some embodiments the ensemble model is a stacking (stacked generalization) model such as a stacked ensemble, or super learner. In some embodiments the ensemble model is a voting ensembles model such as a hard voting classifier (a model where multiple classifiers such as logistic regression, decision trees, and k-NN make predictions, and the final output is determined by a majority vote) or a soft voting classifier (a voting model that averages the probabilities of each class from different models and chooses the class with the highest average probability). In some embodiments a model is a BagBoosting ensemble model (a combination of both bagging and boosting techniques, where the model applies bagging to reduce variance and boosting to reduce bias). In some embodiments the ensemble model is a hybrid ensemble such as a combination of a random forest model and an XGBoost model.

Decision tree models suitable for as a CHIP variant classification model are described in, for example, Duda, 2001, Pattern Classification, John Wiley & Sons, Inc., New York, 395-396, which is hereby incorporated by reference. Decision tree models partition the feature space into a set of rectangles, and then fit a model (like a constant) in each one. In some embodiments, the decision tree is random forest regression. One specific model that can be used to classify a variant as CHIP or not is a classification and regression tree (CART). Other examples of specific decision tree model that can be used as a CHIP variant model include, but are not limited to, ID3, C4.5, MART, and Random Forests. CART, ID3, and C4.5 are described in Duda, 2001, Pattern Classification, John Wiley & Sons, Inc., New York. 396-408 and 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety.

In some embodiments, the model uses any 1, 2, 3, 4, 5, 6, 7, 8, 9, or 10 ten features associated with each variant from the group consisting of gene frequency, alternate allele fragment length median, CHIP likelihood, Kolmogorov-Smirnov test metric for fragment length, p-value for Kolmogorov-Smirnov test metric for fragment length, alternate allele fragment length kurtosis, estimated circulating tumor fraction, ensemble variant allele fraction residual, alternate allele fragment length skew, and variant allele fraction, where such features are further described in Example 3 of the '751 Application. In some embodiments the model uses the following features of a variant to determine whether the variant is a CHIP variant: VAF, polymorphism length, reference and alternate allele fragment length distribution statistics, and historical variant chip likelihood. In some embodiments the model is an ensemble model.

Referring to block 262, in some embodiments, optionally, a second call for MRD is made when there remains a candidate variant in the set of candidate variants after application of the procedure, or a second call against MRD is made when no candidate variant remains in the set of candidate variants after application of the procedure.

Referring to block 263, in some embodiments, an indication is provided that the subject has positive MRD status for the cancer condition when the first call for MRD has been made or an indication that the subject has negative MRD disease status for the cancer condition when the first call against MRD has been made.

Referring to block 264, in some embodiments, the indication that the subject has positive MRD status for the cancer condition is provided when the first call for MRD is made or the second call for MRD is made.

Referring to block 266, in some embodiments, the indication that the subject has negative MRD status for the cancer condition is provided when the first call against MRD and the second call against MRD is made.

Referring to block 268, in some embodiments, a report for the subject is generated that comprises the identity of candidate variants remaining in the set of candidate variants 128 after running the procedure.

Referring to block 270, in some embodiments, the report further comprises a therapeutic match for the subject based on an identity of one or more of the candidate variants remaining in the set of candidate variants after running the procedure.

clinical support for personalized cancer therapy, using the information determined using the first and liquid biopsy sample, as described above. In some embodiments, the report is provided to a patient, physician, medical personnel, or researcher in a digital copy (for example, a JSON object, a pdf file, or an image on a website or portal), a hard copy (for example, printed on paper or another tangible medium). A report object, such as a JSON object, can be used for further processing and/or display. For example, information from the report object can be used to prepare a clinical laboratory report for return to an ordering physician. In some embodiments, the report is presented as text, as audio (for example, recorded or streaming), as images, or in another format and/or any combination thereof.

In some embodiments the report includes information related to the specific characteristics of the patient's cancer condition, e.g., the candidate somatic variants remaining in the candidate variant set after application of the first procedure detailed in FIG. 2, epigenetic abnormalities, associated oncogenic pathogenic infections, and/or pathology abnormalities. In some embodiments, other characteristics of a patient's biological sample and/or clinical records are also included in the report. For example, in some embodiments, the report includes information on the candidate somatic variants 130 remaining in the set of candidate somatic variants after the filtering of such variants in accordance with the process flow of FIG. 2.

In some embodiments, a predicted functional effect and/or clinical interpretation for one or more candidate somatic variants remaining in the candidate somatic variant set after the filtering of such variants in accordance with the process flow of FIG. 2 is curated by using information from variant databases. In some embodiments, a weighted-heuristic model is used to characterize each such candidate somatic variant.

In some embodiments, identified somatic variants are labeled as “potentially actionable”, “biologically relevant”, “variants of unknown significance (VUSs)”, or “benign”. Potentially actionable variants are protein-altering variants with an associated therapy based on evidence from the medical literature. Biologically relevant variants are protein-altering variants that may have functional significance or have been observed in the medical literature but are not associated with a specific therapy. Variants of unknown significance (VUSs) are protein-altering variants exhibiting an unclear effect on function and/or without sufficient evidence to determine their pathogenicity. In some embodiments, benign variants are not reported. In some embodiments, variants are identified through aligning the patient's DNA sequence to the human genome reference sequence version hg19 (GRCh37). In some embodiments, actionable and biologically relevant somatic variants are provided in a clinical summary during report generation.

For instance, in some embodiments, variant classification and reporting is performed, where detected variants are investigated following criteria from known evolutionary models, functional data, clinical data, literature, and other research endeavors, including tumor organoid experiments. In some embodiments, variants are prioritized and classified based on known gene-disease relationships, hotspot regions within genes, internal and external somatic databases, primary literature, and other features of somatic drivers. Variants can be added to a patient (or sample, for example, organoid sample) report based on recommendations from the AMP/ASCO/CAP guidelines. Additional guidelines may be followed. Briefly, pathogenic variants with therapeutic, diagnostic, or prognostic significance may be prioritized in the report. Non-actionable pathogenic variants may be included as biologically relevant, followed by variants of uncertain significance. Evidence may be curated from public and private databases or research and presented as 1) consensus guidelines 2) clinical research, or 3) case studies, with a link to the supporting literature.

In some embodiments, the report includes information about clinical trials for which the subject is eligible, therapies that are specific to the patient's cancer condition, and/or possible therapeutic adverse effects associated with the specific characteristics of the patient's cancer condition, e.g., the patient's genetic variations, epigenetic abnormalities, associated oncogenic pathogenic infections, and/or pathology abnormalities, or other characteristics of the patient's sample and/or clinical records. For example, in some embodiments, the report includes such patient information and analysis metrics, including cancer condition (e.g., cancer type or cancer type and stage) and/or diagnosis, variant allele fraction, patient demographic and/or institution, matched therapies (e.g., FDA approved and/or investigational), matched clinical trials, variants of unknown significance (VUS), genes with low coverage, panel information, specimen information, details on reported variants, patient clinical history, status and/or availability of previous test results, and/or version of bioinformatics pipeline.

In some embodiments, the results included in the report, and/or any additional results (for example, from the bioinformatics pipeline), are used to query a database of clinical data, for example, to determine whether there is a trend showing that a particular therapy was effective or ineffective in treating (e.g., slowing or halting cancer progression), and/or adverse effects of such treatments in other patients having the same or similar characteristics.

In some embodiments, the results of the present disclosure are used to design cell-based studies of the patient's biology, e.g., tumor organoid experiments. For example, an organoid may be genetically engineered to have the same characteristics as the specimen and may be observed after exposure to a therapy to determine whether the therapy can reduce the growth rate of the organoid, and thus may be likely to reduce the growth rate of a cancer condition of the subject. Similarly, in some embodiments, the results are used to direct studies on tumor organoids derived directly from the subject. An example of such experimentation is described in U.S. Pat. No. 11,415,571, the content of which is hereby incorporated by reference, in its entirety, for all purposes.

It should be understood that the examples given above are illustrative and do not limit the uses of the systems and methods described herein in combination with a digital and laboratory health care platform.

The results of the bioinformatics pipeline may be provided for report generation. Report generation may comprise variant science analysis, including the interpretation of variants (including somatic and germline variants as applicable) for pathogenic and biological significance. The variant science analysis may also estimate microsatellite instability (MSI) or tumor mutational burden. Targeted treatments may be identified based on gene, variant, and cancer type, for further consideration and review by the ordering physician. In some aspects, clinical trials may be identified for which the patient may be eligible, based on mutations, cancer type, and/or clinical history. A validation step may occur, after which the report may be finalized for sign-out and delivery. In some embodiments, a first or second report may include additional data provided through a clinical dataflow, such as patient progress notes, pathology reports, imaging reports, and other relevant documents. Such clinical data is ingested, reviewed, and abstracted based on a predefined set of curation rules. The clinical data is then populated into the patient's clinical history timeline for report generation.

Further details on clinical report generation are disclosed in U.S. Pat. No. 10,975,445, which is hereby incorporated by reference.

### III. SECOND EMBODIMENT OF METHODS FOR DETERMINING WHETHER A SUBJECT HAS A POSITIVE OR NEGATIVE MOLECULAR RESIDUAL DISEASE STATUS FOR A CANCER CONDITION

Referring to block 400 of FIG. 4A, systems and methods for determining whether a subject has a positive or negative minimal residual disease (MRD) status for a cancer condition are provided in accordance with another aspect of the present disclosure. In some embodiments the cancer condition is a cancer condition described in block 200 above.

Referring to block 402, in some embodiments, the cancer condition is a particular type of cancer. In some embodiments the cancer condition is a type of cancer described in block 202 above.

Referring to block 404, in some embodiments, the cancer condition is a particular stage of a particular type of cancer. In some embodiments, the cancer condition is a particular stage of a particular type of cancer described in block 204 above.

Referring to block 406, a corresponding nucleic acid sequence of each cell-free DNA fragment in a first plurality of cell-free DNA fragments is obtained from a first plurality of sequence reads of a first sequencing reaction. The first sequencing reaction is a methylation sequencing of the first plurality of cell-free DNA fragments from a first liquid biopsy sample of the test subject. Each respective nucleic acid sequence in the first plurality of nucleic acid sequences comprises a methylation pattern for a corresponding cell-free DNA fragment in the first plurality of cell-free DNA fragments.

In some embodiments, the cell-free DNA is fragmented for the first sequencing reaction. In some such embodiments, the first sequencing reaction of block 206 uses paired-end sequencing so that there are two corresponding sequence reads in the first plurality of sequence reads that encapsulate the sequencing data for each respective cell-free DNA fragment in the first plurality of cell-free DNA fragments.

In some embodiments, personal data corresponding to the second and a record of the one or more biological samples obtained (e.g., patient identifiers, patient clinical data, sample type, sample identifiers, cancer conditions, etc.) are also obtained.

Referring to block 408, in some embodiments, the first liquid biopsy sample is blood. Referring to block 410, in some embodiments, the first liquid biopsy sample comprises blood, whole blood, peripheral blood, plasma, serum, or lymph of the test subject. In some embodiments, one or more of the biological samples obtained from the patient are a biological liquid sample, also referred to as a liquid biopsy sample. In some embodiments, one or more of the biological samples obtained from the patient are selected from blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. In some embodiments, the liquid biopsy sample includes blood and/or saliva. In some embodiments, the liquid biopsy sample is peripheral blood. In some embodiments, one or more blood samples are collected from a subject in commercial blood collection containers. In some embodiments, saliva samples are collected from patients in commercial saliva collection containers.

Referring to block 412, in some embodiments, the volume of the first liquid biopsy sample is less than 30 mL. In some embodiments, the volume of the liquid biopsy sample is from 1 mL to 50 mL, from 2 mL to 40 mL, from 3 mL to 35 mL, or from 5 mL to 31 mL. For example, in some embodiments, the liquid biopsy sample has a volume of about 1 mL, about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, or greater. In some embodiments the liquid biopsy sample is any of the liquid biopsy sample described in block 212. In some embodiments the liquid biopsy sample is obtained in any of the ways described above in block 212.

In some embodiments cell-free DNA is isolated from the liquid biological sample using commercially available reagents, including digestion with proteinase K. In some embodiments, the selective binding properties of a silica membrane are used to extract cell-free DNA from the first liquid biological sample using circulating nucleic acid kits. In some such embodiments, the liquid biological sample is lysed in an optimized buffer and adjusted to binding conditions. Then, the liquid biological sample is loaded directly onto a spin column. In this step, cell-free DNA is bound to the silica membrane, and contaminants are removed in wash steps. Finally, pure cell-free DNA is eluted in small volumes of a low-salt buffer for downstream applications.

In some embodiments, adapters are ligated onto the cell-free DNA fragments that may serve as barcodes to identify which sample is associated with a sequence read, for example, if two or more samples are run together in a sequence. In some embodiments, adapters with barcodes, which are short nucleic acid sequences (e.g., 4-10 base pairs), that identify which nucleic acid molecule is associated with a sequence read, are ligated onto the cell-free DNA fragments. In some embodiments, the adapter comprises degenerate base pairs that serve as a unique tag that can be used to identify sequence reads originating from a specific DNA fragment. In some embodiments, e.g., when multiplex sequencing will be used to sequence cell-free DNA fragments from a plurality of samples (e.g., from the same or different subjects) in a single sequencing reaction, a patient-specific index is also added to the nucleic acid molecules. In some embodiments, the sample specific index is a short nucleic acid sequence (e.g., 3-20 nucleotides) that are added to ends of cell-free DNA fragments during library construction, that serve as a unique tag that can be used to identify sequence reads originating from a specific patient sample.

In some embodiments, an adapter includes a PCR primer landing site, designed for efficient binding of a PCR or second-strand synthesis primer used during the sequencing reaction. In some embodiments, an adapter includes an anchor binding site, to facilitate binding of the cell-free DNA fragment to anchor oligonucleotide molecules on a sequencer flow cell, serving as a seed for the sequencing process by providing a starting point for the sequencing reaction. During PCR amplification following adapter ligation, the UMIs, patient indexes, and binding sites are replicated along with the attached cell-free DNA fragment. This provides a way to identify sequence reads that came from the same original cell-free DNA fragment in downstream analysis.

In some embodiments the sequence reads in the first plurality of sequence reads are trimmed to remove sequencing adapters, amplification primers, and low-quality bases in read ends.

Referring to block 414, in some embodiments, the first sequencing reaction of block 406 is whole genome methylation sequencing or targeted panel sequencing.

Regardless of whether a targeted, probe-based methylation sequencing or a whole genome methylation sequencing is performed, in some embodiments nucleic acids isolated from the biological sample (e.g., cfDNA) are treated to convert unmethylated cytosines to uracils.

In some embodiments, when the nucleic acids are sequenced, cytosines called in the sequencing reaction are methylated, since the unmethylated cytosines are converted to uracils and accordingly would be called as thymidines, rather than cytosines, in the sequencing reaction. In some embodiments commercial kits are used for bisulfite-mediated conversion of methylated cytosines to uracils in the first sequencing reaction of block 406. In some embodiments commercial kits are used for enzymatic conversion of methylated cytosines to uracils in the first sequencing reaction of block 406.

Some embodiments of the first sequencing reaction of block 406 employ the NEB protocol of the NEBNExt enzymatic methyl-seq kit (New England Biolabs, Ipswich, Massachusetts). In some such embodiments, an enzymatic methyl conversion process that oxidizes 5-methylcytosines (5mC) and 5-hydroxymethylcytosines (5hmC) is performed in the first sequencing reaction of block 406. This reaction protects modified cytosines from downstream deamination. TET2 enzymatically oxidizes 5mC and 5hmC through a cascade reaction into 5-carboxycytosine (5-methylcytosine (5mC)=>5-hydroxymethylcytosine (5hmC)=>5-formylcytosine (5fC)=>5-carboxycytosine (5caC)).

In some alternative embodiments, 5hmC is protected from deamination by glucosylation to form 5ghmc using the oxidation enhancer. In some embodiments of the first sequencing reaction of block 406, APOBEC is used to enzymatically deaminate unmodified cytosines to uracils, but does not convert 5caC and 5gmhC. During amplification, uracils are converted to thymines.

In some embodiments, an increased enzymatic ratio in the reaction and/or incubation time, relative to the NEB protocol discussed above, is used to improve the percent of deaminated cytosines in the first sequencing reaction of block 406. For instance, in some embodiments, different ratios of the APOBEC mix (enzyme, buffer & BSA) are used in the first sequencing reaction of block 406. With the published NEB protocol termed 1×, in some embodiments 2× or 3× ratios are used instead as outlined in FIG. 13. In some embodiments a 1.5× or 3.5× ratios is used. In some embodiments an A.Bx ratio is used where A is a real number selected from the range of between 1 and 5 and B is a real number selected from the range between 1 and 25 with the proviso that B is greater than A.

In some embodiments, the cell-free DNA fragments are amplified and purified using commercial reagents.

In some such embodiments, the concentration and/or quantity of the cell-free DNA fragments are quantified using a fluorescent dye and a fluorescence microplate reader, standard spectrofluorometer, or filter fluorometer. In some embodiments, library amplification is performed on a device (e.g., an Illumina C-Bot2) and the resulting flow cell containing amplified cell-free DNA fragments is sequenced.

In some embodiments sequencing is performed on a next generation sequencer (e.g., an Illumina HiSeq 4000, Illumina NovaSeq 6000, Oxford Nanopore, Biomodal) to a unique on-target depth selected by the user. In some embodiments, sequencing is performed using sequencing-by-synthesis technology (Illumina), pyrosequencing (454 Life Sciences), ion semiconductor technology (Ion Torrent sequencing), single-molecule real-time sequencing (Pacific Biosciences), or sequencing by ligation (SOLiD sequencing).

In some embodiments the first sequencing reaction of block 206 is a panel based sequencing reaction.

In some embodiments the first sequencing reaction of block 206 is the panel based sequencing reaction illustrated as sequencing reaction 302 in FIG. 3. In some embodiments the first sequencing reaction of block 206 is a panel based sequencing reaction that makes use of a panel described in Example 6.

In some embodiments the first sequencing reaction of block 406 includes probes for 100 or more, 200 or more, 500 or more, 1000 or more, 1500 or more 2000 or more, 2500 or more, 3000 or more, 3500 or more, 4000 or more, 5000 or more or 10,000 or more differentially methylated loci.

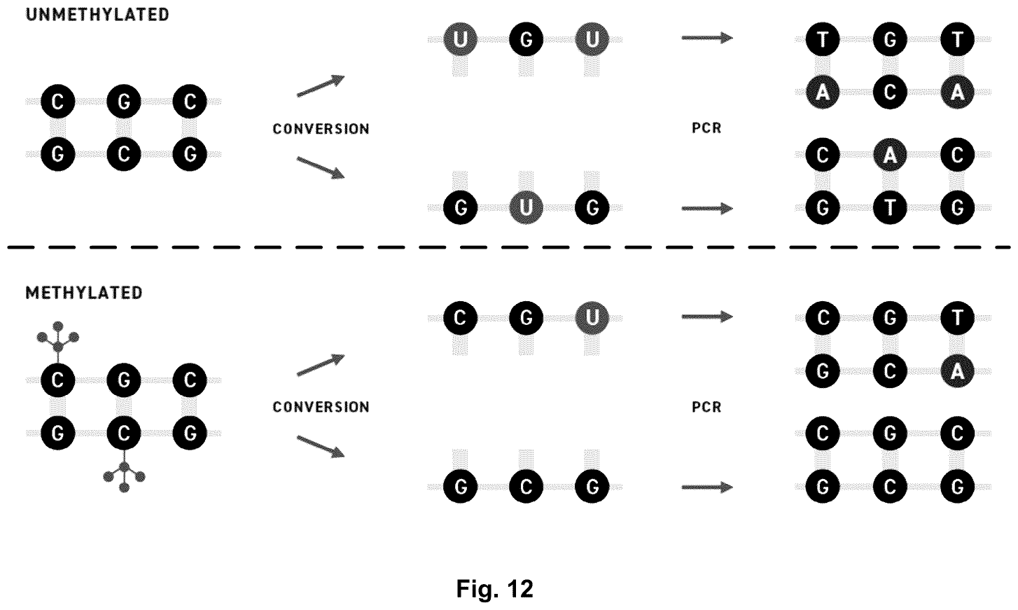

In some embodiments the first sequencing reaction of block 406 includes a plurality of probes. In some embodiments, the plurality of probes is one described in Example 6. In some embodiments, the plurality of loci is one described in Example 6. In some embodiments the plurality of probes includes all four potential sequences at a given loci: methylated, unmethylated, sense and antisense as illustrated in FIG. 12. In some embodiments the plurality of probes includes a first subset that capture methylated sequences for a plurality of loci and a second subset that capture unmethylated sequences for the same plurality of loci. In some embodiments the number of probes in the first subset is equal to the number of probes in the second subset. In alternative embodiments, while the first and second subset of probes each collectively map to the same plurality of loci, the ratio of the number of probes in the first subset to the number of probes in the second subset is other than 1:1. That is, they are mixed at different ratios with emphasis on probes capturing methylated sequences. For instance, in some embodiments there are more probes in the first subset (probes that capture methylated sequences). In some embodiments, the ratio of probes in the first subset to probes in the second subset is 1.25 to 1.00, 1.50 to 1.00, 1.75 to 1.00, 2.00 to 1.00, or 3.00 to 1.00. In some embodiments, the ratio of probes in the first subset to probes in the second subset is X to Y, where X and Y are real positive numbers and X is greater than Y. Without intending to be limited by any particular theory, increasing the concentration of probes in such embodiments enhances methylation detection, reduces the noise from unmethylated normal sequences, and improves assay sensitivity.

Referring to block 416, in some embodiments, the first plurality of sequence reads comprises at least 50,000 sequence reads or at least 250,000 sequence reads.

In some embodiments the first plurality of sequence reads comprises at least 1000, at least 2000, at least 3000, at least 4000, at least 5000, at least 6000, at least 7000, at least 8000, at least 9000, at least 10,000, at least 50,000, at least 100,000, at least 500,000, at least 1 million, at least 2 million, at least 3 million, at least 4 million, at least 5 million, at least 6 million, at least 7 million, at least 8 million, at least 9 million, or more sequence reads. In some embodiments, the first plurality of sequence reads consists of between 50,000 sequence reads and 10 million sequence reads. In some embodiments, the first plurality of sequence reads consists of between 100,000 sequence reads and 8 million sequence reads. In some embodiments, the first plurality of sequence reads consists of between 200,000 sequence reads and 6 million sequence reads.

In some embodiments, the quality of the sequence reads is evaluated (e.g., by interrogating quality metrics like Phred score, base-calling error probabilities, Quality (Q) scores, and the like) and/or removing sequence reads that do not satisfy a threshold quality (e.g., an inferred base call accuracy of at least 80%, at least 90%, at least 95%, at least 99%, at least 99.5%, at least 99.9%, or higher). In some embodiments, the first plurality of sequence reads is filtered based on one or more properties, e.g., removing sequence reads from the first plurality of sequence reads that fail to satisfy a lower or upper size threshold or removing duplicate sequence reads.

Referring to block 417, there is removed, from the first plurality of cell-free DNA, each cell-free DNA that fails to satisfy a methylation rate threshold. Thus, in accordance with block 417, the first plurality of cell-free DNA fragments is filtered to remove each cell-free DNA fragment that fails a CHG context threshold and/or fails to satisfy a methylation rate threshold.

CHG methylation refers to a methylated cytosine that occurs within a CHG trinucleotide context, where H is the IUPAC symbol representing any non-guanine nucleotide (i.e., A, C, or T). In humans, cytosine methylation almost exclusively occurs within CpG contexts, and not within CHG contexts. However, methylated cytosines are still observed within CHG contexts in human sequencing data due to enzymatic failures, such as during NEBNext Enzymatic Methyl-Seq (EM-Seq) library preparation processes. For example, in some instances where the APOBEC enzyme is used for methylation sequencing, the APOBEC enzyme fails to properly deaminate an unmethylated cytosine into a uracil, that cytosine nucleotide is read as a “C” during sequencing, which causes it to be erroneously classified as a methylated cytosine. Though this failure mode is easiest to identify among CHG contexts, which are not naturally methylated in humans, APOBEC failure can occur in any genomic context, including CpGs.

While methylation sequencing errors, such as APOBEC failure, at any grain is considered undesirable, it may especially confound the models of the present disclosure when they lead to fragment level failures. Fragment-level APOBEC failure represents the case in which cytosines of any context on a fragment appear methylated due to complete or near-complete failure of the APOBEC enzyme during the enzymatic conversion step for that fragment. This is particularly confounding because the signal used in the present disclosure is derived from fragments that exhibit high degrees of CpG methylation among the differentially methylated regions (DMRs) specified by the model. Consequently, a fragment that presents with many methylated CpGs is equally likely to be classified as tumor-derived whether the observed hypermethylation occurred as a product of legitimate biological processes or fragment-level methylation sequencing failure.

To mitigate the risk of artifactually methylated fragments contributing to the signal used in the present disclosure, a pre-processing filter that specifically targets and removes from the first plurality of cell-free DNA fragments “unconverted fragments,” or fragments that have experienced widespread unmethylation cytosine deamination failure is run. Because CpG methylation can be both biologically- or artifactually-derived in humans, the rate at which CpG methylation is observed (methylation rate) on a given fragment may not inform whether unmethylation cytosine deamination failure arose. The term “methylation rates” in the context of CHG methylation refers to the frequency or proportion of cytosines in the CHG context that are methylated in a given DNA fragment. Given that CHG methylation does not occur naturally in humans, and can hence be confidently deemed an artifact, unconverted fragment identification relies entirely on the rate at which CHG methylation is observed on a DNA fragment (methylation rate).

Referring to block 418, in some embodiments, the methylation rate threshold is satisfied by a cell-free DNA fragment when: i) the cell-free DNA fragment has fewer than a threshold number of CHG sites, or ii) the cell-free DNA fragment has the threshold number of CHG sites or greater, and a CHG methylation rate that is less than a threshold methylation rate.

For example, in some embodiments, cell-free DNA fragments that contain five or more CHG contexts and exhibit CHG methylation rates in excess of 80% are deemed “unconverted,” or likely to have been seriously affected by methylation sequencing failure, such as APOBEC failure. Accordingly, in some embodiments these cell-free DNA fragments are removed from the first plurality of cell-free DNA fragments prior to further processing to both reduce confounding factors presented to the models of the present disclosure and to mitigate the risk of artifactual signal generation.

In some embodiments, the methylation rate threshold is satisfied by a cell-free DNA fragment when the cell-free DNA fragment has at least one CHG site that is methylated.

In some embodiments, the methylation rate threshold is satisfied by a cell-free DNA fragment when the cell-free DNA fragment has at least two CHG sites that are methylated.

In some embodiments, the methylation rate threshold is satisfied by a cell-free DNA fragment when the cell-free DNA fragment has at least 3, 4, 5, 6, 7, or 8 CHG sites that are methylated.

Referring to block 420, in some embodiments, the threshold number of CHG sites is five CHG sites and the threshold methylation rate is eighty percent.

Referring to block 422, in some embodiments, the threshold number of CHG sites is between 3 and 50 CHG sites and the threshold methylation rate is between 3 percent and 98 percent. In some embodiments the threshold number of CHG sites is 3, 4, 5, 6, 7, 8, 9, 10, 11, 12, 13, 14, 15, 16, 17, 18, 19, or 20 sites.

Referring to block 424, in some embodiments, the threshold number of CHG sites is between 6 and 25 CHG sites. In some embodiments the threshold methylation rate is between 12 percent and 82 percent. In some embodiments the threshold methylation rate is between 5 percent and 95 percent. In some embodiments the threshold methylation rate is between 5 percent and 10 percent, between 10 percent and 15 percent, between 15 percent and 20 percent, between 20 percent and 25 percent, between 25 percent and 30 percent, between 30 percent and 35 percent, between 35 percent and 40 percent, between 40 percent and 45 percent, between 45 percent and 50 percent, between 50 percent and 55 percent, between 55 percent and 60 percent, between 60 percent and 65 percent, between 65 percent and 70 percent, between 70 percent and 75 percent, between 75 percent and 80 percent, between 80 percent and 85 percent, between 85 percent and 90 percent, or between 90 percent and 95 percent. In some embodiments, the threshold methylation rate is 5, 10, 15, 20, 25, 30, 35, 40, 45, 50, 55, 60, 65, 70, 75, 80, 85, 90, or 95 percent.

Referring to block 426, a determination is made of a corresponding number of unexpected circulating-tumor (ctDNA) fragments mapping to each respective region in a plurality of regions of one or more first reference sequences of the species of the test subject (e.g., using a methylation pattern of each nucleic acid sequence in the first plurality of nucleic acid sequences). In some embodiments in accordance with block 426 the plurality of regions of one or more first reference sequences of the species of the test subject are defined as described in block 217 above.

In some embodiments the plurality of regions consists of between 10 and 250 different regions of the one or more reference sequences. In some embodiments the plurality of regions consists of between 20 and 300 different regions of the one or more reference sequences. In some embodiments the plurality of regions comprises 10 or more regions, 30 or more regions, 40 or more regions, 60 or more regions, 80 or more regions, or 100 or more regions of the one or more reference sequences.

In some embodiments, the plurality of regions consists of between 100 and 2500 different regions of the one or more reference sequences. In some embodiments, the plurality of regions consists of between 200 and 3000 different regions of the one or more reference sequences. In some embodiments, the plurality of regions comprises 100 or more regions, 300 or more regions, 400 or more regions, 600 or more regions, 800 or more regions or 10,000 or more regions of the one or more reference sequences.

In some embodiments the plurality of regions consists of between 1000 and 25,000 different regions of the one or more reference sequences. In some embodiments the plurality of regions consists of between 2000 and 30,000 different regions of the one or more reference sequences. In some embodiments the plurality of regions comprises 1000 or more regions, 3000 or more regions, 4000 or more regions, 6000 or more regions, 8000 or more regions or 10,000 or more regions of the one or more reference sequences.

Mapping cell-free DNA fragments to the plurality of regions. To perform block 426, each of the cell-free DNA fragments in the first plurality of cell-free DNA fragments that were not filtered out in block 416 are mapped onto the one or more first reference sequences in order to ascertain which regions in the plurality of regions the fragments map to. In some embodiments in accordance with block 426, the cell-free DNA fragments are mapped to the one or more first reference sequences of the species of the test subject using a mapping program. A nonlimiting example of a mapping program is Bismark (version 0.14.2) with default parameters. See Krueger and Andrews, 2011, “Bismark: a flexible aligner and methylation caller for Bisulfite-Seq applications,” Bioinformatics 27(11), pp. 1571-1572, which is hereby incorporated by reference. Another nonlimiting example of a mapping program is BWAMeth. See Pederson, 2014, “Fast and accurate alignment of long bisulfite-seq reads,” arXiv:1401.1129 [q-bio.GN]

In some embodiments in accordance with block 426, one or more alignment algorithms map the first plurality of cell-free DNA fragments to one or more first reference sequences, e.g., a reference genome, exome, or targeted-panel construct. Additional algorithms for mapping nucleic acid sequences of cell-free DNA fragments to one or more first reference sequences are known in the art, for example, Burrows-Wheeler Alignment (BWA), Blat, SHRiMP, LastZ, and MAQ. One example of a mapping package is the BWA tool, which uses a Burrows-Wheeler transform to map short sequence reads against one or more first reference sequences, allowing for mismatches and gaps. See, 2009, Li and Durbin, Bioinformatics, 25(14), pp. 1754-60, the content of which is incorporated herein by reference, in its entirety, for all purposes. In some embodiments cell-free DNA fragments are mapped to the one or more first reference sequences by mapping the sequence reads associated with such cell-free DNA fragments from the first sequencing reaction. In some embodiments cell-free DNA fragments are mapped to the one or more first reference sequences by mapping the sequences of the cell-free DNA fragments directly.

In some embodiments in accordance with block 426, the sequences of the first plurality of cell-free DNA fragments are mapped to the plurality of regions based on genomic coordinates.

In some embodiments, any cell-free fragment that overlaps any portion of any region 112 is mapped to that region. In such embodiments, the mapping occurs at the fragment context and not the site context.

In some embodiments, if regions are proximal to each other on the one or more first reference sequences and a single cell-free DNA fragment overlaps with multiple regions, that cell-free DNA fragment is mapped to each of the multiple regions.

Once each respective cell-free DNA fragment in the first plurality of cell-free DNA fragments has been mapped to a particular one or more regions in the plurality of regions, a model that makes use of the methylation pattern of the respective cell-free DNA fragment is used to predict whether the cell-free DNA fragment is an unexpected cell-free fragment or not. In other words, once the first plurality of cell-free DNA fragments has been mapped to the plurality of regions, the methylation pattern of each respective cell-free DNA fragment is used to assign a probability that the respective cell-free DNA fragment is an unexpected circulating-tumor DNA fragment. An unexpected circulating-tumor DNA fragment is one that has a methylation pattern that would not be expected in a cancer free subject.

In some embodiments, the methylation pattern of a respective cell-free DNA fragment in the first plurality of cell-free DNA fragments that has been mapped to a given region in the plurality of regions is used to determine a methylation configuration of the respective cell-free DNA fragment. In some embodiments, this is done by counting (i) how many CpG sites are on the portion of the respective cell-free DNA fragment mapping to the given region (Y_{total_cpg_sites}) and, of these, (ii) how many are methylated (X_{cpg_sites_methylated}). It will be appreciated that Y_{total_cpg_sites} will be a subset of the CpG sites on the respective cell-free DNA fragment (if the respective cell-free DNA fragment is partially overlapping a region) or all the CpG sites on the fragment (if the cell-free DNA fragment is wholly contained within the given region).

In some embodiments, the methylation configuration (Y_{total_cpg_sites}, X_{cpg_sites_methylated}) of the respective cell-free DNA fragment together with the identity of the region the fragment maps to is used to determine whether the cell-free DNA fragment should be designated an unexpected circulating-tumor DNA fragment or not. In some embodiments, this is done by using the methylation configuration and the identity of the region it maps to look up the probability that the cell-free DNA fragment is derived from the tumor cells. In some embodiments, such probabilities are available for several different methylation configurations for each region in the plurality of regions. That is, for each methylation configuration in the plurality of methylation configurations for a respective region, a probability between 0 and 1 is provided. Example 1 provides a non-limiting example of the construction of such probabilities for several different methylation configurations for each region in a plurality of regions.

In some embodiments, a cut-off is applied to the probability returned for a respective cell-free DNA fragment based on its methylation configuration and region 110 mapping identity. In some embodiments the cut-off is 0.9. For example, if the look up for a respective cell-free DNA fragment based on its methylation configuration and region 110 returns a probability of 0.89 that the DNA fragment is derived from tumor (is an unexpected circulating-tumor DNA fragment), and the cutoff is 0.9, the respective cell-free DNA fragment is not labeled an unexpected circulating-tumor DNA fragment. As another example, if the look up for a respective cell-free DNA fragment based on its methylation configuration and region 110 returns a probability of 0.91 that the DNA fragment is derived from tumor, and the cutoff is ninety percent, the respective cell-free DNA fragment is labeled an unexpected circulating-tumor DNA fragment. In some embodiments, a different threshold is applied for making this cut off decision, such as a value between 0.7 percent and 0.99, a value between −0.75 and 0.90, or a value between 0.80 and 0.95. In some embodiments the cut off value is 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, or 0.99.

Through the mapping of each respective cell-free DNA fragment in the first plurality of cell-free DNA fragments to respective region in the plurality of regions and the labeling of such mapped fragments based on their mapping and their methylation profiles a corresponding number of unexpected circulating-tumor DNA fragment mapping to each respective region 112 in the plurality of regions of one or more first reference sequences 110 of the species of the test subject is made using the methylation patterns of the cell-free DNA fragment.

Referring to block 428, an unexpected fragments per million value is determined for the first liquid biopsy sample from the corresponding number of unexpected ctDNA fragments in each respective region of the plurality of regions of the one or more first reference sequences observed in the first liquid biopsy sample. For instance, in some embodiments this is done by normalizing the corresponding number of unexpected circulating-tumor (ctDNA) fragments in each region. For example, if the unexpected circulating-tumor (ctDNA) fragments in a particular region is 20 and 20,000 fragments in the first plurality of cell-free DNA fragments mapped to the particular region, the normalized unexpected circulating-tumor (ctDNA) fragments per million in the particular region is

$$( \frac{20}{20 , 000} ) \times {10}^{6} = 10 , 0.$$

Referring to block 429, the corresponding number of unexpected circulating-tumor DNA (ctDNA) fragments mapping is used to determine an unlikely fragments per million value across the plurality of regions. To obtain an unlikely fragments per million value across the plurality of regions an aggregation function is used. For example, in some embodiments a measure of central tendency is taken of the unlikely fragments per million value for each region in the plurality of regions. In some embodiments the maximum unlikely fragments per million value observed for any region in the plurality of regions serves as the unlikely fragments per million value across the plurality of regions. In some embodiments the minimum unlikely fragments per million value observed for any region in the plurality of regions serves as the unlikely fragments per million value across the plurality of regions.

Referring to block 430, a first threshold is applied to the unlikely fragments per million value to provide a first call for molecular residual disease when the unlikely fragments per million value satisfies the first threshold or a first call against molecular residual disease when the unlikely fragments per million value fails to satisfy the first threshold.

In some embodiments, the first threshold is associated with the maximal amount of circulating tumor fraction that can be detected and tolerated when calling a sample negative for the cancer condition. In this instance, it can be referred to as a limit of blank threshold. In some embodiments, the first threshold value will vary from batch to batch of subjects. In some embodiments the first threshold is determined by any approved or standardized clinical and laboratory procedures and guidelines, for example a nonparametric method.

In some embodiments the first threshold for the unlikely fragments per million value is calculated as a limit of blank (LOB) or a limit of detection (LOD) for circulating tumor fraction in an analysis of biological samples from subjects that are free of the cancer condition measured according to the Clinical and Laboratory Standards Institute guidelines EP17.2 and relevant reports. See, for example, Perry et al., Evaluation of Detection Capability for Clinical Laboratory Measurement Procedures; Approved Guideline—Second Edition Volume 32 Number 8; and Wang et al., 2019, “KRAS Mutant Allele Fraction in Circulating Cell-Free DNA Correlates with Clinical Stage in Pancreatic Cancer Patients,” Front. Oncol (9), 1295, each of which is hereby incorporated by reference.

Because the first threshold is based on evaluation of subjects free of the cancer condition, a precise fixed value for the threshold is dependent on the subjects analyzed to determine the threshold. However, by way of a non-limiting example, consider the case where the threshold is 0.05 and the unlikely fragments per million value is 0.07. In this example, since the unlikely fragments per million value of 0.07 exceeds the first threshold value of 0.05, the first threshold is satisfied and a first call for molecular residual disease is made. In another non-limiting example, consider the case where the first threshold is 0.05 and the unlikely fragments per million value is 0.04. In this example, since the unlikely fragments per million value of 0.04 is less than the first threshold value of 0.05, the first threshold is not satisfied and a first call against molecular residual disease is made. In some embodiments, unlikely fragments per million values are on the order of tens or one hundreds.

Referring to block 432, in some embodiments an indication is provided that the subject has positive molecular residual disease status for the cancer condition when the first call for molecular residual disease is made, or an indication is provided that the subject has negative molecular residual disease status for the cancer condition when the first call against molecular residual disease.

Referring to block 434, in some embodiments, there is optionally obtained, from a second sequencing reaction, a corresponding sequence of each cell-free DNA fragment in a second plurality of cell-free DNA fragments in a second liquid biopsy sample of the test subject, thereby obtaining a second plurality of sequence reads.

In some embodiments, the second sequencing reaction is a duplex sequencing reaction.

In some embodiments the second plurality of sequence reads is acquired by any methodology known in the art. For example, next generation sequencing (NGS) techniques such as sequencing-by-synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), or nanopore sequencing (Oxford Nanopore Technologies) is performed. In some embodiments sequencing is performed by next generation sequencer (e.g., an Illumina HiSeq 4000, Illumina NovaSeq 6000, etc.). In some such embodiments this sequencing is a paired-end sequencing is performed. In some embodiments, massively parallel sequencing is performed using sequencing-by-synthesis with reversible dye terminators. In some embodiments, sequencing is performed using next generation sequencing technologies, such as short-read technologies. In other embodiments, long-read sequencing or another sequencing method known in the art is used.

In some embodiments the second sequencing reaction is NGS that produces hundreds of thousands, millions, or hundreds of millions of sequence reads from the second plurality of cell-free DNA fragments in the biological sample (e.g., after fragmentation of the cell-free DNA). Accordingly, in some embodiments, the plurality of sequence reads obtained by NGS of cell-free DNA fragments are DNA sequence reads. In some embodiments, the sequence reads in the second plurality of sequence reads have an average length of at least fifty nucleotides. In other embodiments, the sequence reads in the second plurality of sequence reads have an average length of at least 50, 60, 70, 80, 90, 100, 150, 200, 250, 300, or more nucleotides.

In some embodiments, the second sequencing reaction is performed after enriching for nucleic acids (e.g., cfDNA, gDNA, and/or RNA) encompassing a plurality of predetermined target sequences, e.g., human genes and/or non-coding sequences associated with cancer in accordance with block 230 discussed in further detail below. Advantageously, sequencing a nucleic acid sample that has been enriched for target cell-free DNA fragments, rather than all cell-free DNA fragments isolated from the second biological sample, significantly reduces the average time and cost of the second sequencing reaction. Accordingly, in some embodiments, the methods described herein include obtaining a second plurality of sequence reads of cell-free DNA fragments that have been hybridized to a probe set for hybrid-capture enrichment (e.g., of one or more genes listed in Table 1 above).

Referring to block 436, in some embodiments, the first liquid biopsy sample and the second liquid biopsy sample are the same liquid biopsy sample. For instance, a common liquid biopsy sample is obtained from the subject and then split into two aliquots, one for the first sequencing reaction and the other for the second sequencing reaction.

Referring to block 438, in some embodiments, the first liquid biopsy sample and the second liquid biopsy sample are different liquid biopsy samples.

Referring to block 440, in some embodiments, the second sequencing reaction is a panel-based sequencing reaction of a plurality of loci.

In some embodiments, the second sequencing reaction is a panel-based sequencing reaction of a plurality of loci. In some such embodiments a plurality of nucleic acid probes (e.g., a probe set) is used to enrich one or more target sequences in the second liquid biopsy sample for the plurality of loci. In some embodiments, the probe set includes probes targeting one or more gene loci, e.g., exon or intron loci within the plurality of loci. In some embodiments, the plurality of loci enriched by the probe set includes one or more loci not encoding a protein, e.g., regulatory loci, miRNA loci, and other non-coding loci, e.g., that have been found to be associated with cancer. In some embodiments, the plurality of loci includes at least 25, 50, 100, 150, 200, 250, 300, 350, 400, 500, 750, 1000, 2500, 5000, or more human genomic loci.

In some embodiments, the plurality of loci includes one more of the genes listed in Table 1. In some embodiments, the plurality of loci includes at least 5 of the genes listed in Table 1. In some embodiments, the plurality of loci includes at least 10 of the genes listed in Table 1. In some embodiments, the plurality of loci includes at least 25 of the genes listed in Table 1. In some embodiments, the plurality of loci includes at least 50 of the genes listed in Table 1. In some embodiments, the plurality of loci includes at least 75 of the genes listed in Table 1. In some embodiments, the plurality of loci includes at least 100 of the genes listed in Table 1. In some embodiments the plurality of loci consists of or comprises all of the genes listed in Table 1.

Generally, probes for enrichment of nucleic acids (e.g., cfDNA obtained from a liquid biopsy sample) include DNA, RNA, or a modified nucleic acid structure with a base sequence that is complementary to a locus of interest. For instance, a probe designed to hybridize to a locus in a cell-free DNA fragment can contain a sequence that is complementary to either strand, because the cell-free DNA fragments are double stranded. In some embodiments, each probe in the plurality of probes includes a nucleic acid sequence that is identical or complementary to at least 10, at least 11, at least 12, at least 13, at least 14, or at least 15 consecutive bases of a locus in the plurality of loci. In some embodiments, each probe in the plurality of probes includes a nucleic acid sequence that is identical or complementary to at least 20, 25, 30, 40, 50, 75, 100, 150, 200, or more consecutive bases of a locus in the plurality of loci.

In some embodiments, the plurality of probes is one described in Example 6.

In some embodiments, the plurality of loci is one described in Example 6.

In some embodiments the plurality of probes includes all four potential sequences at a given loci: methylated, unmethylated, sense and antisense. In some embodiments the plurality of probes includes a first subset that capture methylated sequences and a second subset that capture unmethylated sequences. In some embodiments the number of probes in the first subset is equal to the number of probes in the second subset. In alternative embodiments, while the first and second subset of probes each collectively map to the same number of loci, the ratio of the number of probes in the first subset to the number of probes in the second subset is other than 1:1. That is, they are mixed at different ratios with emphasis on probes capturing methylated sequences. For instance, in some embodiments there are more probes in the first subset (probes that capture methylated sequences). In some embodiments, the ratio of probes in the first subset to probes in the second subset is 1.25 to 1.00, 1.50 to 1.00, 1.75 to 1.00, 2.00 to 1.00, or 3.00 to 1.00. In some embodiments, the ratio of probes in the first subset to probes in the second subset is X to Y, where X and Y are real positive numbers and X is greater than Y. Without intending to be limited by any particular theory, increasing the concentration of probes in such embodiments enhances methylation detection, reduces the noise from unmethylated normal sequences and improves the assay sensitivity.

In some embodiments, the plurality of loci is a whole-exome panel comprising the exomes of a biological sample. In some embodiments, the plurality of loci is a whole-genome panel that comprises the genome of a specimen.

In some embodiments, the probes for the second sequencing region include additional nucleic acid sequences that do not share any homology to the plurality of loci. For example, in some embodiments, the probes also include nucleic acid sequences containing an identifier sequence, e.g., a unique molecular identifier (UMI), e.g., that is unique to a particular sample or subject. Similarly, in some embodiments, the probes also include primer nucleic acid sequences useful for amplifying the nucleic acid molecule of interest, e.g., using PCR. In some embodiments, the probes also include a capture sequence designed to hybridize to an anti-capture sequence for recovering the nucleic acid molecule of interest from the sample.

Likewise, in some embodiments, the probes each include a non-nucleic acid affinity moiety covalently attached to a nucleic acid molecule that is complementary to a locus in the plurality of loci, for recovering a cell-free DNA fragment. Non-limited examples of non-nucleic acid affinity moieties include biotin, digoxigenin, and dinitrophenol. In some embodiments, the probe is attached to a solid-state surface or particle, e.g., a dip-stick or magnetic bead, for recovering the nucleic acid of interest. In some embodiments, the methods described herein include amplifying the nucleic acids that bound to the probe set prior to further analysis, e.g., sequencing. Methods for amplifying nucleic acids, e.g., by PCR, are well known in the art.

In some embodiments, panel-targeting sequencing is performed to an average on-target depth of at least 500×, at least 750×, at least 1000×, at least 2500×, at least 500×, at least 10,000×, or greater depth.

Referring to block 442, in some embodiments, the plurality of loci is sequenced at an average sequence depth of at least 250× by the second sequencing reaction. Referring to block 444, in some embodiments, the plurality of loci is sequenced at an average sequence depth of at least 1000× by the second sequencing reaction. In some embodiments, the plurality of loci is sequenced at an average sequence depth of at least 500×, at least 750×, at least 1000×, at least 2500×, at least 500×, at least 10,000×, or greater.

Referring to block 446, in some embodiments, the second plurality of sequence reads comprises at least 50,000 sequence reads or at least 250,000 sequence reads. In some embodiments the second plurality of sequence reads comprises at least 1000, at least 2000, at least 3000, at least 4000, at least 5000, at least 6000, at least 7000, at least 8000, at least 9000, at least 10,000, at least 50,000, at least 100,000, at least 500,000, at least 1 million, at least 2 million, at least 3 million, at least 4 million, at least 5 million, at least 6 million, at least 7 million, at least 8 million, at least 9 million, or more sequence reads. In some embodiments, the second plurality of sequence reads consists of between 50,000 sequence reads and 10 million sequence reads. In some embodiments, the second plurality of sequence reads consists of between 100,000 sequence reads and 8 million sequence reads. In some embodiments, the second plurality of sequence reads consists of between 200,000 sequence reads and 6 million sequence reads.

In some embodiments each respective sequence read in the second plurality of sequence reads is mapped to one or more second reference sequences (e.g., a reference human genome) by identifying a sequence in a region of the one or more second reference sequences that best matches the sequence of nucleotides in the respective sequence read. In some embodiments, this mapping uses methods known in the art to determine alignment position information. The alignment position information may indicate a beginning position and an end position of a region in the one or more second reference sequences that corresponds to a beginning nucleotide base and end nucleotide base of the respective sequence read. Alignment position information may also include the sequence read length, which can be determined from the beginning position and end position. A region in the one or more reference sequences may be associated with a gene or a segment of a gene. Any of a variety of alignment tools can be used to optimize the mapped alignment.

For instance, local sequence alignment algorithms compare subsequences of different lengths in the sequence read to subsequences in the one or more reference sequences to create the best alignment for each portion of the query sequence. In contrast, global sequence alignment algorithms align the entirety of the sequence read.

In some embodiments, the sequence read mapping process starts by building an index of either the one or more reference sequences or the second plurality of sequences reads, which is then used to retrieve the set of positions in the one or more reference sequences where a sequence read is more likely to align. Once this subset of possible mapping locations has been identified, alignment is performed in these candidate regions with slower and more sensitive algorithms. See, for example, Hatem et al., 2013, “Benchmarking short sequence mapping tools,” BMC Bioinformatics 14: p. 184; and Flicek and Birney, 2009, “Sense from sequence reads: methods for alignment and assembly,” Nat Methods 6(Suppl. 11), S6-S12, each of which is hereby incorporated by reference. In some embodiments, the mapping methodology makes use of a hash table or a Burrows-Wheeler transform (BWT). See, for example, Li and Homer, 2010, “A survey of sequence alignment algorithms for next-generation sequencing,” Brief Bioinformatics 11, pp. 473-483, which is hereby incorporated by reference.

Other software programs designed to align sequence reads to reference sequences include, but are not limited to, Novoalign (Novocraft, Inc.), Bowtie, Burrows Wheeler Aligner (BWA), and/or programs that use a Smith-Waterman algorithm. Example reference sequences includes genomes such as, for example, hg19, GRCh38, hg38, GRCh37, and/or other reference genomes developed by the Genome Reference Consortium.

In some embodiments, each respective sequence read in the second plurality of sequence reads is in FASTQ file format and is aligned to a location in the one or more second reference sequences (e.g., a human genome) having a sequence that best matches the sequence of nucleotides in the respective sequence read. There are many software programs designed to map sequence reads, for example, Novoalign (Novocraft, Inc.), Bowtie, Burrows Wheeler Aligner (BWA), programs that use a Smith-Waterman algorithm, etc. Alignment may be directed using a reference genome (for example, hg19, GRCh38, hg38, GRCh37, other reference genomes developed by the Genome Reference Consortium, etc.) by comparing the nucleotide sequences in each read with portions of the nucleotide sequence in the reference genome to determine the portion of the reference genome sequence that is most likely to correspond to the sequence in the read. In some embodiments, one or more SAM files are generated for the alignment, which store the locations of the start and end of each read according to coordinates in the reference genome and the coverage (number of reads) for each nucleotide in the reference genome. The SAM files may be converted to BAM files. In some embodiments, the BAM files are sorted and duplicate reads are marked for deletion, resulting in de-duplicated BAM files.

In some embodiments, adapter-trimmed FASTQ files are aligned to the 19th edition of the human reference genome build (HG19) using Burrows-Wheeler Aligner (BWA) [PMC2705234]. Following alignment, the sequence reads are grouped by alignment position and UMI family and collapsed into consensus sequences, for example, using fgbio tools (http://fulcrumgenomics.github.io/fgbio/). Bases with insufficient quality or significant disagreement among family members (for example, when it is uncertain whether the base is an adenine, cytosine, guanine, etc.) may be replaced by N's to represent a wildcard nucleotide type. PHRED scores are then scaled based on initial base calling estimates combined across all family members. Following single-strand consensus generation, duplex consensus sequences are generated by comparing the forward and reverse oriented PCR products with mirrored UMI sequences. In various embodiments, a consensus can be generated across read pairs. Otherwise, single-strand consensus calls will be used. Following consensus calling, in some embodiments, filtering is performed to remove low-quality consensus fragments. In some embodiments, the consensus fragments are then re-aligned to the human reference genome using BWA. In some embodiments, a BAM output file is generated after the re-alignment, then sorted by alignment position, and indexed.

In some embodiments, the sequencing data is normalized, e.g., to account for pull-down, amplification, and/or sequencing bias (e.g., mappability, GC bias etc.).

Referring to block 448, in some embodiments, the second liquid biopsy sample is blood.

Referring to block 450, in some embodiments, the second liquid biopsy sample comprises blood, whole blood, peripheral blood, plasma, serum, or lymph of the subject.

Referring to block 452, in some embodiments, the one or more first reference sequences is a human reference genome, the one or more second reference sequences is the human reference genome, the second plurality of loci comprises 1000 or more loci cumulatively mapping to between four megabases and ten megabases of the human reference genome, the first sequencing reaction is a first panel-based sequencing reaction of a first plurality of loci, and the first plurality of loci comprises 50 or more loci cumulatively mapping to between 0.1 megabase and 1 megabase of the human reference genome.

Referring to block 453, in some embodiments, optionally, the second plurality of sequence reads is used to identify each candidate somatic variant in a set of candidate somatic variants, where each candidate somatic variant in the set of candidate somatic variants is a single nucleic acid variant (SNV).

In some embodiments, optionally, the second plurality of sequence reads is used to identify each candidate somatic variant in a set of candidate somatic variants, where each candidate somatic variant in the set of candidate somatic variants is a single nucleotide variant (SNV), a multi-nucleotide variant (MNV), an indel (e.g., an insertion or deletion of nucleotides), a DNA rearrangement (e.g., an inversion or translocation of a portion of a chromosome or chromosomes), a variation in the copy number of a locus (e.g., an exon, gene, or a large span of a chromosome) (CNV), a partial or complete change in the ploidy of the cell, or an epigenetic variant (e.g., analtered DNA methylation patterns).

In some embodiments, the set of candidate somatic variants is obtained by evaluating sequence reads in the second plurality of sequence reads that map to any one of the following genes: AKT1, ALK, APC, AR, ARID1A, ATM, B2M, BRAF, BRCA2, CCND1, CDKN2A, CTNNB1, ERBB2, FBXW7, FGFR2, FGFR3, FGFR4, GNA11, GNAQ, HNF1A, IDH1, JAK2, KDR, KIT, KMT2A, KRAS, MAP2K1, MAP2K2, MSH3, MSH6, MTOR, NF1, NOTCH1, NRAS, NTRK1, PBRM1, PIK3CA, PIK3R1, PTCH1, PTEN, RAF1, RET, RNF43, SMAD4, SMO, TERT, and TP53. In some such embodiments the cancer condition is colon cancer. In some such embodiments the cancer condition is a cancer other than colon cancer. In some embodiments the cancer condition is a cancer condition listed in block 202.

In some embodiments, the set of candidate somatic variants is obtained by evaluating sequence reads in the second plurality of sequence reads that map to any one of a collection of genes, where the collection of genes comprises at least 5, 6, 7, 8, 9, 10, 11, 12, 13, 14, 15, 16, 17, 18, 19, or 20, 25, 30, or 35 of the following genes: AKT1, ALK, APC, AR, ARID1A, ATM, B2M, BRAF, BRCA2, CCND1, CDKN2A, CTNNB1, ERBB2, FBXW7, FGFR2, FGFR3, FGFR4, GNA11, GNAQ, HNF1A, IDH1, JAK2, KDR, KIT, KMT2A, KRAS, MAP2K1, MAP2K2, MSH3, MSH6, MTOR, NF1, NOTCH1, NRAS, NTRK1, PBRM1, PIK3CA, PIK3R1, PTCH1, PTEN, RAF1, RET, RNF43, SMAD4, SMO, TERT, and TP53. In some such embodiments the cancer condition is colon cancer. In some such embodiments the cancer condition is a cancer other than colon cancer. In some embodiments the cancer condition is a cancer condition listed in block 202.

In some embodiments, the set of candidate somatic variants is obtained by evaluating sequence reads in the second plurality of sequence reads that map to any one of a collection of genes, where the collection of genes consists of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 or all 47 of the following genes: AKT1, ALK, APC, AR, ARID1A, ATM, B2M, BRAF, BRCA2, CCND1, CDKN2A, CTNNB1, ERBB2, FBXW7, FGFR2, FGFR3, FGFR4, GNA11, GNAQ, HNF1A, IDH1, JAK2, KDR, KIT, KMT2A, KRAS, MAP2K1, MAP2K2, MSH3, MSH6, MTOR, NF1, NOTCH1, NRAS, NTRK1, PBRM1, PIK3CA, PIK3R1, PTCH1, PTEN, RAF1, RET, RNF43, SMAD4, SMO, TERT, and TP53. In some such embodiments the cancer condition is colon cancer. In some such embodiments the cancer condition is a cancer other than colon cancer. In some embodiments the cancer condition is a cancer condition listed in block 202.

In some embodiments, the set of candidate somatic variants is obtained by evaluating sequence reads in the second plurality of sequence reads that map to any one of a collection of genes, where the collection of genes consists of the genes set forth in Table. 1. In some such embodiments the cancer condition is colon cancer. In some such embodiments the cancer condition is a cancer other than colon cancer. In some embodiments the cancer condition is a cancer condition listed in block 202.

Referring to block 454, in some embodiments, each respective candidate somatic variant in the set of candidate somatic variants is identified by applying a variant caller to the second plurality of sequence reads with a restriction that the variant caller determines that each respective candidate somatic variant in the set of candidate somatic variants has a variant allele frequency of at least 0.1 in the second plurality of sequence reads and that at least one cell-free DNA fragment in the second plurality of cell-free DNA fragments exhibits the respective candidate somatic variant. In some embodiments, identification of block 454 results in the identification of a candidate somatic variant set that comprises 10 or more, 15 or more, 20 or more, 25 or more, 30 or more, 35 or more, 40 or more, 45 or more, 50 or more, 100 or more, 200 or more, 300 or more, 400 or more, 500 or more, 1000 or more, 2000 or more, 3000 or more, 4000 or more, 5000 or more or 10,000 or more candidate somatic variants. In some embodiments, each candidate somatic variant maps to a different portion of the genome of a species.

In some embodiments, each respective candidate somatic variant in the set of candidate somatic variants is identified by applying a variant caller to the second plurality of sequence reads with a restriction that the variant caller determines that each respective candidate somatic variant in the set of candidate somatic variants has a variant allele frequency of at least 0.1 in the second plurality of sequence reads and that at least one cell-free DNA fragment in the second plurality of cell-free DNA fragments exhibits the respective candidate somatic variant. As used herein, the term “variant allele frequency,” “VAF,” “allelic fraction,” or “AF” refer to the number of times a variant or mutant allele was observed in the second plurality of sequence reads (e.g., a number of reads supporting a variant allele) divided by the total number of times the position the variant or mutant allele occupies was sequenced (e.g., a total number of reads covering a candidate locus).

In some embodiments, candidate somatic variant detection is accomplished using VarDict (available on the internet at github.com/AstraZeneca-NGS/VarDictJava). Both SNVs and INDELs are called and then sorted, deduplicated, normalized and annotated. In some embodiments, the annotation uses SnpEff to add transcript information, 1000 genomes minor allele frequencies, COSMIC reference names and counts, ExAC allele frequencies, and Kaviar population allele frequencies. The annotated variants are then classified as germline, somatic, or uncertain using a Bayesian model based on prior expectations informed by databases of germline and cancer variants. In some embodiments, uncertain variants are treated as somatic for filtering and reporting purposes.

Referring to block 456, in some embodiments, optionally the set of candidate somatic variants is filtered by a procedure.

Referring to block 458, in some such embodiments, there is removed from the set of candidate somatic variants those respective candidate somatic variants in the set of candidate somatic variants that are present in a repository of known germline variants.

In some embodiments, variants are present in a repository of known germline variants when they are represented in the population above a threshold level, e.g., as determined using a population database such as ExAC or gnomAD. For instance, in some embodiments, variants that are represented in at least 1% of the alleles in a population are annotated as germline in the repository. In other embodiments, variants that are represented in at least 2%, at least 3%, at least 4%, at least 5%, at least 7.5%, at least 10%, or more of the alleles in a population are annotated as germline in the repository. In some embodiments, sequencing data from a matched sample from the subject, e.g., a normal tissue sample, is used to annotate variants identified in the second liquid biopsy sample from the subject. That is, variants that are present in both the second liquid biopsy sample and a normal sample represent those variants that were in the germline and can be annotated in the repository as germline variants.

Referring to block 460, in some embodiments, the procedure comprises removing from the set of candidate somatic variants each respective candidate somatic variant in the set of candidate somatic variants that maps to a variant interval in a plurality of variant intervals, where each respective variant interval in the plurality of variant intervals is identified as having a pre-test odds of a positive variant call that is less than a pre-test odds threshold value based upon a prevalence of a corresponding one or more training variants, which are each above a limit of detection and map to the respective variant interval, in a plurality of tumor-normal matched samples for the cancer condition obtained from a first cohort of training subjects having the cancer condition with the proviso that no variant detected in a second cohort of healthy samples maps to the respective variant interval. Such a filtering step is disclosed in further detail in block 246 in conjunction with Example 3. Referring to block 462, in some embodiments, the pre-test odds threshold value is 0.001.

Referring to block 464, in some embodiments, remove from the set of candidate somatic variants each respective candidate somatic variant in the set of candidate somatic variants that is identified as an artifactual variant. More details on such a filter are disclosed in block 250.

Referring to block 466, in some embodiments, the procedure comprises removing from the set of candidate allele variants each respective candidate variant in the set of candidate allele variants in which the second sequencing reaction produced a coverage depth of less than a threshold amount for the respective locus in one or more second reference sequences of the species of the subject that the candidate somatic variant maps to.

In some embodiments a minimum of 250× sequencing depth must be observed for the locus a candidate somatic variant in order to retain the candidate somatic variant. In some embodiments, a minimum 50×, 100×, 150×, 200×, 300×, 350×, 400×, 450×, 500×, 1000×, or 2000× sequencing depth must be observed for the locus a candidate somatic variant in the second sequencing reaction maps to in order to retain the candidate somatic variant. Such sequencing depths in accordance with block 252 refer to the actual locus in the one or more second reference sequences that the candidate somatic variant maps to, as opposed to the overall depth of the second sequencing reaction.

In some embodiments, the threshold sequencing depth is a duplex sequencing depth in accordance with the definition given for duplex sequencing depth in the definitions section above. In some such embodiments, the locus a candidate somatic variant maps to must have at least a 50×, 100×, 150×, 200×, 300×, 350×, 400×, 450×, 500×, 1000×, or 2000× duplex sequencing depth in the second sequencing reaction in order to be retained as a candidate somatic variant.

In considering the threshold sequencing depth of block 252, not all of the cell-free DNA fragments mapping to the locus that a given candidate somatic variant maps to need to have the mutant allele. For instance, in the case where the coverage depth threshold amount is 50×, 49 cell-free DNA fragments having the wild-type allele and one cell-free DNA fragment mapping to the locus that the given candidate somatic variant maps to would be sufficient to meet the coverage requirements.

In some embodiments, there is a requirement that, in order to retain a given candidate somatic variant in the set of given candidate somatic variants, both strands of at least one cell-free nucleic acid fragment bearing the mutant (alt) allele be identified from (supported by) the second sequencing reaction. Example 5 provides one example of how this duplex consensus read support can affect specificity and sensitivity. In some embodiments, there is a requirement that, in order to retain a given candidate somatic variant in the set of given candidate somatic variants, both strands of at least 2, 3, 4, or 5 cell-free nucleic acid fragments bearing the mutant (alt) allele of the given candidate somatic variant be identified from (supported by) one or more sequence reads of the second plurality of sequence reads of the second sequencing reaction.

Referring to block 468, in some embodiments, remove from the set of candidate somatic variants each respective candidate somatic variant in the set of candidate somatic variants that (a) maps to a repeat region in the one or more second reference sequences of the species and (b) is not annotated as a known somatic mutation in a database of known somatic mutations for the species of the subject. Repeat regions are repeating sequences of two or more base pairs that are adjacent to one another and are abundant throughout genomes, such as the human genome. See, for example, Madsen et al., 2008, “Short tandem repeats in human exons: a target for disease mutations,” BMC genomics, 9, p. 410, which is hereby incorporated by reference. Databases of known somatic mutations for humans include, but are not limited to, ClinVar and COSMIC. See, Landrum et al., 2020, “ClinVar: improvements to accessing data,” Nucleic Acids Res. 48(D1), pp. D835-D844; and Tate et al., “COSMIC: the Catalogue of Somatic Mutations in Cancer,” Nucleic Acids Research 47 D1, pp. D941-D947, each of which is hereby incorporated by reference. Thus, in some embodiments, if a candidate somatic variant maps to a repeat region and is not in ClinVar it is removed from the set of candidate somatic variants. In some embodiments, if a candidate somatic variant maps to a repeat region and is not in COSMIC it is removed from the set of candidate somatic variants. In some embodiments, if a candidate somatic variant maps to a repeat region and is not in COSMIC or in ClinVar it is removed from the set of candidate somatic variants. ClinVar and COSMIC are nonlimiting examples of databases of known somatic mutations and other databases, or any combination of such databases may be used for condition (b) of block 254.

Referring to block 470, in some embodiments, remove from the set of candidate somatic variants each respective candidate somatic variant in the set of candidate somatic variants that maps to a region of clonal hematopoiesis of indeterminate potential (CHIP). Such embodiments are employed to mitigate the risk that a CHIP variant is passed as a candidate somatic variant. Further description of CHIP is provided in conjunction with block 256.

Referring to block 472, in some embodiments, the region of clonal hematopoiesis of indeterminate potential (CHIP) is ASXL1, BCOR, BCORL1, CBL, CREBBP, CUX1, DNMT3A, GNB1, JAK2, PPM1D, PRPF8, SETDB1, SF3B1, SRSF2, TET2, U2AF1, or any combination thereof. In some embodiments any subset of these genes is used for the CHIP filter. In some embodiments this set of genes, or any subset thereof, is used for the CHIP filter when the cancer condition is a BRCA-associated cancer. See Marshall et al., “Germline mutations and the presence of clonal hematopoiesis of indeterminate potential (CHIP) in 20,963 patients with BRCA-associated cancers.,” DOI: 10.1200/JCO.2023.41.16_suppl.10522 Journal of Clinical Oncology 41, no. 16_suppl (Jun. 1, 2023) 10522-10522, which is hereby incorporated by reference.

In some embodiments, the procedure comprises removing from the set of candidate somatic variants 128 each respective candidate somatic variant 130 in the set of candidate somatic variants that maps to TET2, DNMT3A, ASXL1, or SF3B1. In some embodiments any subset set of these genes is used for the CHIP filter of block 256. In some embodiments this set of genes, or any subset thereof, is used for the CHIP filter when the cancer condition is a BRCA-associated cancer.

Referring to block 474, in some embodiments, the procedure comprises removing from the set of candidate somatic variants each respective candidate somatic variant in the set of candidate somatic variants that maps to TET2, DNMT3A, ASXL1, SF3B1, or any combination thereof. In some embodiments any subset set of these genes is used for the CHIP filter.

Referring to block 476, in some embodiments, the region of CHIP is TET2, DNMT3A, ASXL1, SF3B1, CBL, U2AF1, IDH2,2,3, MYD88,13, EP300, CDKN2C, HNF1A, or any combination thereof. In some embodiments any subset set of these genes is used for the CHIP filter.

In some embodiments, the procedure comprises removing from the set of candidate somatic variants each respective candidate somatic variant in the set of candidate somatic variants that maps to TET2, DNMT3A, ASXL1, SF3B1, CBL, U2AF1, IDH2,2,3, MYD88,13, EP300, CDKN2C, or HNF1A when the subject is 70 years of age or older. Such genes are known CHIP genes in subjects of this age. See, Severson, 2018, “Detection of clonal hematopoiesis of indeterminate potential in clinical sequencing of solid tumor specimens,” Blood 131(22), pp. 2501-2505, which is hereby incorporated by reference.

Referring to block 478, in some embodiments, the procedure further comprises removing from the set of candidate somatic variants each respective candidate somatic variant in the set of candidate somatic variants that fails to be represented by at least one cell-free DNA fragment in the second plurality of cell-free DNA fragments in which both strands of the at least one cell-free DNA fragment are identified in one or more sequence reads of the second plurality of sequence reads.

Referring to block 480, in some embodiments, the procedure further comprising removing from the set of candidate allele variants each candidate variant in the set of candidate allele variants that has a variant allele fraction exceeding an upper threshold fraction in the second plurality of sequence reads.

Referring to block 482, in some embodiments, the procedure further comprises removing from the set of candidate allele variants each candidate variant in the set of candidate allele variants that is observed in a cohort of healthy subjects.

Referring to block 483, in some embodiments, optionally, a second call for minimal residual disease is made when there remains a candidate variant in the set of candidate variants after application of the procedure, or a second call against minimal residual disease when no candidate variant remains in the set of candidate variants after application of the procedure.

Referring to block 484, in some embodiments an indication is provided that the subject has positive minimal residual disease status for the cancer condition when the first call for minimal residual disease has been made or an indication that the subject has negative minimal residual disease status for the cancer condition when the first call against minimal residual disease has been made.

Referring to block 486, in some embodiments, the indication that the subject has positive minimal residual disease status for the cancer condition is provided when the first call for minimal residual disease is made or the second call for minimal residual disease is made.

Referring to block 488, in some embodiments, the indication that the subject has negative minimal residual disease status for the cancer condition is provided when the first call against minimal residual disease and the second call against minimal residual disease is made.

Referring to block 490, in some embodiments, a report is generated for the subject comprising the identity of candidate variants remaining in the set of candidate variants after running the procedure.

Referring to block 492, in some embodiments, the report further comprises a therapeutic match for the subject based on an identity of one or more of the candidate variants remaining in the set of candidate variants after running the procedure.

Further details of such a report are provided in block 270.

### IV. EXAMPLES

### Example 1—Construction of Probabilities for Several Different Methylation Configurations for Each Region 112 in the Plurality of Regions

Sample groups were created for model training. Each sample group was composed of three in vivo generated samples and a series of derived in silico samples. For each respective subject represented by a sample group, the sample group contains a pool of (i) a clinical plasma (cell-free DNA) from the respective subject, (ii) a solid tumor (biopsy) sample from the respective subject, and (iii) a normal plasma control sample form an age, gender, and race matched normal plasma control. From these samples the sequence reads from the normal plasma (iii) and the clinical plasma (i) were mixed (in-silico) to create a titer series of known and pre-determined circulating-tumor DNA values for use in lower limit of detection evaluation.

Within a sample group, models were trained on the normal plasma (i) and solid tumor components (ii) as well as a limited number of NA12878 cell line samples. The NA12878 cell line is derived from a healthy donor and thus can serve as a proxy for normal.

An upfront step to ensure use of regions 112 that have similar methylation patterns over their fragments between normal plasma and NA12878 cell line was performed. This unlocks the ability to use cell lines in place of normal plasma for certain validation studies. To this end, a median methylation rate was computed on a per sample class (normal plasma versus NA12878 cell line) per region 112 in the plurality of regions over all fragments in the pool. Thus, for each sample class, for each region, a median methylation rate was determined from the cell-free DNA fragment in the pool. These median methylation rates were used to identify a subset of the plurality of regions having functional equivalence. A region was defined as having functional equivalent when the median fragment methylation rate across sample classes was within a tolerance of each other. In this example the tolerance was 1%. Thus, if the median methylation rate for a given region 112 in the plurality of regions computed using the cell-free DNA fragments from the normal plasma was within 1% of the median methylation rate for the same given region 112 using the cell-free DNA fragments from the normal plasma NA12878 cell line, the given region was included in the subset of cell lines. In some embodiments, another tolerance value is used, such as 0.02, 0.03, 0.04, 0.05, or a value between 0.01 and 0.20.

In some embodiments, those regions 112 that exceeded the tolerance were removed from the plurality of regions 112 and were not used in computing the excess fragments per million value of block 220 or the corrected excess fragments per million value of block 22. In some embodiments, the NA12878 cell line filtering step was not performed and regions were not removed from the plurality of regions based on such filtering.

With the regions 112 selected, a per region probability matrix that discriminates between a circulating tumor DNA fragment and a healthy tissue derived DNA fragment was created in order to provide a probability, for any given cell-free DNA fragment mapping to one of these regions, of being circulating-tumor DNA.

The methylation configuration (Y_{total_cpg_sites}, X_{cpg_sites_methylated}) of each cell-free fragment for each biological sample from a sample group was determined as described in block 217 in each of the segments for each class (normal plasma, solid tumor). These raw counts were converted to counts per million for depth normalization. A first three dimensional matrix for each sample in the Normal Plasma class was created. A second three dimensional matrix for each sample in the Solid Tumor class was also created. The dimensions of each matrix were X: number of CpG Sites on a cell-free DNA fragment within the region, given an upper limit per region, Y: number of those CpG sites that are methylated, Z: sample dimension, one entry of Z per sample.

For each matrix (sample class), aggregation over the samples represented by the matrix was performed using an aggregation metric, e.g. minimum, maximum, measure of central tendency, etc.). Moreover, the method of aggregation can be the same or different for each matrix (sample class). In the present example, mean was used for aggregation and then a two-dimensional kernel density estimating smoothing was applied over the resulting two-dimensional matrix. This matrix now represents per sample class the density of the observed configurations over all the methylation configurations within the region. This process generates the PMₙₒᵣₘₐₗ and PM_{solid_tumor} per region 112.

These probability matrices (PM) per sample class were combined for ctDNA probability calling. In some embodiments, each sample class specific probability matrix is normalized to one by dividing each value in the matrix by the sum of the matrix and then combined to provide the probability of origination from tumor (and therefore a circulating tumor fragment) using the following formula: [PM_{solid_tumor}]/([PMₙₒᵣₘₐₗ]+[PM_{solid_tumor}]). For each methylation configuration for a given region, the formula provides a probability that the given methylation configuration is derived specifically from the solid tumor sample (which should represent the fragment methylation configurations of the circulating-tumor DNA). For example if a particular fragment has 5 CpG of which four were methylated and the methylation configuration for the region the particular fragment maps to has a value of 0.8 in PM_{solid_tumor} and 0.1 in PMₙₒᵣₘₐₗ the formula [PM_{solid_tumor}]/([PMₙₒᵣₘₐₗ]+[PM_{solid_tumor}]) reads out 0.88, (0.8/[0.8+0.1]), which is an 88% chance of being a circulating-tumor DNA derived fragment. Conversely, if both values are 0.5 the result would be 0.50, (0.5/1[0.5+0.5]) meaning a 50% change being a circulating-tumor DNA derived fragment.

### Example 2—Selection of Regions 112

This example describes how regions used in downstream computation of the excess fragments per million value (block 220) and the corrected excess fragments value (block 222) are computed in some embodiments. An emission rate is computed for all regions under consideration (candidate regions) using the per sample class parameters determined in Example 1. In some embodiments, each candidate region is a genomic region whose differential methylation state has been associated with cancer. See, for example, Skvortsova, “The DNA methylation landscape in cancer,” Essays Biochem 63(6), pp. 797-811, which is hereby incorporated by reference, discloses approaches for identifying such genomic regions and exemplary regions in humans. See also Biswas et al., 2017, “Epigenetics in cancer: Fundamentals and beyond,” Pharmacol. Ther. 173, pp. 118-134, which is hereby incorporated by reference.

In an example in accordance with block 206, each candidate region corresponds to one of the approximately 12 k differentially methylated regions (DMRs) in a commercially available pan-cancer methylation probe panel. For each sample used in training (sample 1), probabilities are assigned to each fragment within each candidate region for which PM_{solid_tumor} and PMₙₒᵣₘₐₗ values have been constructed in Example 1. This results in a probability of being circulating-tumor DNA for every fragment. All the fragments above a given threshold (e.g., 0.9, meaning ninety percent) were defined as unlikely to be derived from normal tissue. The percent of unlikely fragments over total fragments becomes the emission rate for a sample in a marker region. In some embodiments, a different threshold value is used. In some embodiments, a different threshold is applied for making this cut off decision, such as a value between 0.70 and 0.99, a value between 0.75 and 0.90, or a value between 0.80 and 0.95. In some embodiments the cut off value is 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, or 0.99.

Next, the emission rate fold change from normal to tumor was computed for each region. Candidate regions that were found to have different methylation status between normal and tumor tissues were selected as the plurality of regions. For each candidate region, the median emission rate for each sample class (normal plasma or solid tumor) was used to create a ratio to compute fold change [mER_{solid_tumor}]/([mER_{normal_plasma}]+le⁻⁵) per candidate region. The candidate regions were ranked based on this metric in descending order.

In one instance, the top ranked N regions (e.g., 100 for the model used in this example) were selected as the plurality of regions used to compute the excess fragments per million value in accordance with block 220 and the corrected excess fragments per million value in accordance with block 222. The calculations of example 1 and this example indicate that these regions have the large difference in emission rates between solid tumor and normal biopsies and thus are a suitable place to look for fragments with differential methylation signals.

In another instance, the top ranked 250 regions were selected as the plurality of regions used to compute the excess fragments per million value in accordance with block 220 and the corrected excess fragments per million value in accordance with block 222. The calculations of example 1 and this example indicate that these regions have the large difference in emission rates between solid tumor and normal biopsies and thus are a suitable place to look for fragments with differential methylation signals.

### Example 3—Identification of High Confidence Variant Intervals

Example 3 illustrates block 246, in which a corresponding threshold for a respective candidate somatic variant is determined. The corresponding threshold is for the respective locus in one or more second reference sequences of the species of the subject that the candidate somatic variant maps to. The corresponding threshold is based upon at least a pre-test odds of a positive variant call for the respective locus based upon a prevalence of variants (variant interval) in a genomic region that includes the respective locus in a cohort of training subjects having the cancer condition.

For a given locus, a pre-test odds (PreTestOdds) was computed using the formula:

$$PreTestOdds = PreTestProbability / ( 1 - PreTestProbability )$$

Here, the pre-test probability (PreTestProbability) is the probability of having a positive variant given the patient cancer type and prevalence of the variant. The pre-test probability was calculated from the frequency of observed variants in historical xT tumor-normal matched data at the locus.

Somatic, filter-passing (remaining after performing the filters described in blocks 244, 250, 252, 256) single-nucleotide variants (SNVs) with a variant allele frequency above the xT limit of detection (Beaubier et al., “Clinical validation of the tempus xT next-generation targeted oncology sequencing assay,” Oncotarget 10(24) 2384-2396, which is hereby incorporated by reference) in 8,674 xT tumor-normal matched data that intersect the xFv2 panel (Table 1) were used to calculate the xF for MRD pre-test-odds. In other words, the above formula was used to compute a pre-test odds, for each SNP that was both (i) above the xT limit of detection in the xT tumor-normal matched data and (ii) that mapped to a gene in the xFv2 panel (Table 1). Out of the 8,674 xT tumor-normal matched CRC Stage I-IV patients used for training, a total of 49,043 SNVs were identified.

The 49,043 SNVs were organized into SNV variant intervals. If SNVs were found adjacent to each other, they were grouped into the same variant interval. If a SNV was not adjacent to another SNV it received its own variant interval. This yielded, from the original 49,043 SNVs, a total of 17,732 variant intervals, with variant intervals ranging between 1-7 bp in length. For each respective SNV variant interval in the 17,732 variant intervals, SNV prevalence was calculated for the SNV variant interval by dividing the number of SNVs in the 49,043 SNVs detected in the respective variant interval by the total number of xT samples.

Not all of the 17,732 variant intervals called using xT may be specific to MRD. Therefore, normal samples were utilized to identify which of the 17,732 variant intervals includes variants detected in healthy subjects. Any variant interval in the 17,732 variant intervals that had more than 1 variant detected in the normal samples (either after filtering using the criteria of blocks 244, 250, 252, 256 or not filtering at all) were removed from consideration as an MRD interval. This filtered the 17,732 variant intervals called using xT down to a total of 12,292 MRD variant intervals with pre-test-odds (CRC for MRD pre-test odds) ranging from 0.000115 to 0.454 and variant intervals continued to range between 1-7 bp in length.

The CRC for MRD pre-test-odds for the 12,292 MRD variant intervals was used to run a Bayesian dynamic variant filter that uses an application of Bayes' Theorem. having the form:

$$specificity = 1 - preTestOdds \times sensitivity / posrtTestOdds$$

Here, the post-test-odds value (postTestOdds) is fixed and the preTestOdds is the CRC for MRD pre-test odds for a particular variant interval in the 12,292 MRD variant intervals. The post-test-odds is calculated from the post-test probability (which is the probability of having a positive variant given Bayes Theorem) and is pre-defined (e.g., 50 percent). The sensitivity is the fraction of variants detected by the xFv2 assay at 0.1%, 0.25%, and 0.5% VAF or greater. The sensitivity is based on the xFv2 LOD validation in healthy subjects. The derived specificity value for any given MRD interval can then be used in a quantile BetaBinomial function to derive the minimum number of alternate alleles that can be observed at a given depth.

Of the 12,292 MRD variant intervals, 287 high confidence MRD variant intervals with a pre-test-odds >=0.001 were identified. It was found that approximately 94.4% of the training samples contained at least 1 SNV within a high confidence MRD. The sensitivity of calling variants in high-confidence MRD variant intervals (>=0.001) differs from the sensitivity of calling variants in low pre-test odds intervals (Table 2). Utilizing a post-test-odds (pto) of 1 yielded the best sensitivity (Table 2).

TABLE 2
Variant calling sensitivity with pre-test-odds.

| Post-test | Alt Read | Pre-test- | 1000x | 2500x | 5000x |
|---|---|---|---|---|---|
| probability | Support | odds | Coverage | Coverage | Coverage |
| 50% (pto = 1) | 1 | >=0.001 | 54.28% | 14.73% | 0.0% |
| | | <0.001 | 3.37% | 0.05% | 0.0% |
| | 2 | >=0.001 | 88.87% | 39.04% | 14.73% |
| | | <0.001 | 35.01% | 0.83% | 0.05% |
| | 3 | >=0.001 | 98.8% | 62.84% | 28.77% |
| | | <0.001 | 79.77% | 7.02% | 0.28% |

The training tumor and normal samples were run through the xM-variant workflow to assess the variant filtering without a multiplier (Table 3). Tumor and normal samples were also assessed for sensitivity and specificity.

TABLE 3
Variant calling performance with CRC variant-level pre-test-odds ..

| | | | True Positive | |
|---|---|---|---|---|
| | | Sample- | MRD+ tumor | False Positive |
| Sample | Sample-level | level | samples VAF | MRD+ normal |
| cohort | Sensitivity | Specificity | Range | samples |
| Training | 75.87% | 100% | 0.033%-92.77% | 0%-0% |
| | (n = 489) | (n = 20) | | |

### Example 4—Modified Variant Filtering Criteria

Supplemental variant filtering criteria were added in this example after the filtering of Example 3 as set forth below. As noted above, Example 3 is an example of the filtering of block 246 in which high confidence variant allele intervals are identified.

Likely germline variants with a variant allele frequency greater than 40% were removed from the set of candidate variants. The disclosed pipeline labels variants as somatic or germline using a Bayesian model based on prior expectations informed by internal and external databases of germline and cancer variants as described in Example 3. However, germline variants may still be misclassified as somatic in some instances. To further exclude possible germline variants, all candidate somatic variants must also have a VAF less than 40% in order to be retained in the set of candidate somatic variants in this example.

All candidate somatic variants in the set of candidate somatic variants must pass variant filters for likely germline variants (block 244), artifact filtering (block 250), VAF (less than 40%), and duplex consensus read support of at least one cell-free DNA fragment. However, to retain candidate somatic variants with as low variant allele frequencies as possible, a number of filters were not used to filter the candidate somatic variants in the set of candidate somatic variants. An overview of the variant annotations that may filter a variant and that are not used in Example 4, and various embodiments of the present disclosure, include the following.

A) a dynamic variant threshold that may filter SNVs lacking alternate allele support in the presence of likely high background sequencing error. Accordingly, in some embodiments, this dynamic variant threshold is not used in order to retain candidate somatic variants at as low variant allele frequencies as possible.

B) NM5.25, p8, pSTD: VarDict variant filters for mean mismatches in reads (NM5.25), mean position in reads is less than 8 (p8), and the standard deviation of the position in reads is 0 (pSTD). Accordingly, in some embodiments, these filtering parameters are not used in order to retain candidate somatic variants at as low variant allele frequencies as possible.

C) Blacklist and greylist: filters frequently observed artifactual variants (blacklist) and variants observed in healthy subjects (greylist). Accordingly, in some embodiments, this filter, further described in block 254, is not used in order to retain candidate somatic variants at as low variant allele frequencies as possible.

D) A minimum of 250× sequencing depth must be observed for the SNV. Accordingly in some embodiments, this filter is not used in order to retain candidate somatic variants at as low variant allele frequencies as possible.

E) Homopolymer: filters variants that intersect a repeat region and is not an established somatic mutation observed in public variant databases, such as ClinVar and COSMIC. Accordingly, in some embodiments this filter, further described in block 254, is not used in order to retain candidate somatic variants at as low variant allele frequencies as possible.

F) Application of a filter for candidate artifactual variants in MSI-high regions that have a low signal to noise ratio. Accordingly, in some embodiments this filter, further described in block 254, is not used in order to retain candidate somatic variants at as low variant allele frequencies as possible.

To further improve specificity, variants in non-high confidence MRD variant intervals were not used to make the MRD+/− call. For instance, in some embodiments those candidate variant alleles mapping to the plurality of variant intervals that failed to achieve a pre-test-odds greater than or equal to 0.001 as described in Example 3 and further described above in conjunction with block 246 were removed from the set of candidate variant alleles.

The inclusion of the additional variant filtering criteria of (i) mapping to high confidence variant intervals (Example 3 and block 246), (ii) likely germline variants (block 244), (iii) artifact filtering (block 250), and (iv) VAF less than 40% removed possible germline variants and SNPs that were likely artificial variants as set forth in Table 4.

TABLE 4
Sample-level specificity and sensitivity in MRD variant
calling in accordance with block 246 as implemented in
Example 3 with the additional variant filtering
criteria set forth in Example 4 that were not excluded from use.

| | | Sample- | TP MRD+ tumor | FP MRD+ |
|---|---|---|---|---|
| Sample | Sample-level | level | samples VAF | normal |
| cohort | Sensitivity | Specificity | Range | samples |
| Training | 67.48% | 100% | 0.034%-39.98% | 0%-0% |
| | (n = 489) | (n = 20) | | |

### Example 5—Duplex Read Support Criteria

Molecular tags added to cfDNA allow for the identification of either single consensus or duplex consensus mutations. Duplex consensus reads supporting a mutation offer higher confidence that the mutation is not an artifact since the mutation is found on both strands of the double stranded DNA. In the MRD pipeline in accordance with Example 4, the ratio of duplex alternate reads (number of duplex reads supporting the alt allele/total reads supporting the alt allele) for the second plurality of sequence reads was calculated. Here, “alt allele” means the variant.

Requiring at least 1 duplex consensus read supporting the alternate allele reduces the number of false positive SNVs passing MRD filters (Table 5).

TABLE 5
Sample-level specificity and sensitivity in MRD variant calling with the addition
of the criterion of a duplex consensus read requirement to the criteria of Example 4
((i) mapping to high confidence variant intervals (Example 3 and block 246), (ii) likely
germline variants (block 244), (iii) artifact filtering (block 250), and (iv) VAF less than 40%).

| | | | True Positive | |
|---|---|---|---|---|
| | | Sample- | MRD+ tumor | False Positive |
| Sample | Sample-level | level | samples VAF | MRD+ normal |
| cohort | Sensitivity | Specificity | Range | samples |
| Training | 65.44% | 100% | 0.036%-39.98% | 0%-0% |
| | (n = 489) | (n = 20) | | |

Tumor and normal samples were held out from training (n=201× Fv2 historical tumor samples and n=40 normal xFv2 Streck/Roche normals). These testing samples (Testing dataset) were run through the above-described workflow and sensitivity and specificity were assessed in Table 6.

TABLE 6
Sample-level specificity and sensitivity in MRD variant calling with the addition of
the criterion of a duplex consensus read requirement to the criteria of Example 4
((i) mapping to high confidence variant intervals (Example 3 and block 246), (ii) likely
germline variants (block 244), (iii) artifact filtering (block 250), and (iv)
VAF less than 40%) or the testing dataset.

| | | | | True Positive | |
|---|---|---|---|---|---|
| | | | | MRD+ tumor | False Positive |
| Sample | Variant-level | Sample-level | Sample-level | samples VAF | MRD+ normal |
| cohort | Specificity | Sensitivity | Specificity | Range | samples |
| Testing | Default (no | 74.13% | 97.5% | 0.054%-39.83% | 0%-0.79% |
| | multiplier) | (n = 201) | (n = 40) | | |

### Example 6: Exemplary Sequencing Regions

In some embodiments, the plurality of regions for block 214 or block 406 comprises 1.5 megabases of the one or more first reference sequences that form an exemplary pan-cancer methylation probe panel, for example, a panel that is commercially available. This panel is a custom target enrichment panel focusing specifically on targets relevant to 31 different cancers. The panel provides coverage of clinically focused targets that allow for the study of methylation patterns relevant to cancer detection and diagnosis from tumor and liquid biopsy samples. The panel covers 31 cancer types, 47 disease entities, and is based on the methylation patterns observed between subjects with particular cancer conditions and subjects free of particular cancer conditions in the TCGA database. See TCGA Research Network, available on the Internet at cancer.gov/tcga. The panel makes use of approximately 13K probes (for sequencing the panel at high depth in some embodiments in accordance with the first sequencing reaction of block 206), encompasses 126 k CpG sites, and approximately 12 k differentially methylated regions (DMRs). In some embodiments, each of the 12 k differentially methylated regions of the pan-cancer methylation panel is a corresponding region in the plurality of regions of the one or more reference sequences of block 216.

In some embodiments, the plurality of regions for block 217 or block 426 comprises a customized panel (V1p1) that involves the addition of stable and differential regions as well as consistently hypermethylated regions in addition to an exemplary methylation panel to bring the panel size from 1.5 Mb to 10 Mb bases (V1p1), with over 50,000 probes. In some embodiments, the probes include all four potential sequences at each site (loci): methylated, unmethylated, sense and antisense.

### Example 7: Model Performance in Accordance with the Embodiment of FIG. 2

In this example, the performance of a model in accordance with an embodiment drawn from FIG. 2 was tested. The model was run for each of 70 subjects having early stage colorectal cancer (stage 2 or 3) in order to determine whether each of the subjects had a positive or negative molecular residual disease status for the cancer condition. Each of the subjects underwent curative surgery.

Four weeks after the curative surgery, a blood sample was collected from each subject. A corresponding nucleic acid sequence of each cell-free DNA fragment in a first plurality of cell-free DNA fragments, from a first plurality of sequence reads of a first sequencing reaction was obtained for each subject using their blood sample. The first sequencing reaction was a methylation sequencing of the first plurality of cell-free DNA fragments from the blood sample of the subject. Each respective nucleic acid sequence in the first plurality of nucleic acid sequences comprised a methylation pattern for a corresponding cell-free DNA fragment in the first plurality of cell-free DNA fragments for each subject.

A corresponding number of circulating-tumor DNA (ctDNA) fragments was determined for each subject by mapping the fragments to each respective region in a plurality of regions of a human reference genome using a methylation pattern of each nucleic acid sequence in the first plurality of nucleic acid sequences.

A corresponding expected number of noise fragments in each respective region of the plurality of regions of the one or more first reference sequences of the species of the subject was determined based on a corresponding distribution using an observed sequencing depth from the first sequence reaction and a learned background emission rate for the respective region for each subject.

An excess fragments per million value for the first liquid biopsy sample from the corresponding number of ctDNA fragments in each respective region of the plurality of regions of the one or more first reference sequences observed in the first liquid biopsy sample in excess of the corresponding expected number of noise fragments in the respective region, was determined for each of the subjects.

The excess fragments per million value for the first liquid biopsy sample for each of the subjects was corrected by an observed CHG methylation level to obtain a corrected excess fragments per million value.

A first threshold was applied to the corrected excess fragments per million value to provide, for each of the subjects, a first call for molecular residual disease when the corrected excess fragments per million value satisfied the first threshold or a first call against molecular residual disease when the corrected excess fragments per million value failed to satisfy the first threshold.

In addition to the first call, a second call was made for each of the subjects. To make the second call, there was obtained, from a second sequencing reaction, for each of the subjects, a corresponding sequence of each cell-free DNA fragment in a second plurality of cell-free DNA fragments from the above-described blood sample of the subject, thereby obtaining a second plurality of sequence reads.

For each of the subjects, the second plurality of sequence reads was used to identify each candidate somatic variant in a set of candidate somatic variants, where each candidate somatic variant in the set of candidate somatic variants was a single nucleic acid variant (SNV). In this example, the sequence reads for genes mapping to the following genes were searched for the set of candidate somatic variants: AKT1, ALK, APC, AR, ARID1A, ATM, B2M, BRAF, BRCA2, CCND1, CDKN2A, CTNNB1, ERBB2, FBXW7, FGFR2, FGFR3, FGFR4, GNA11, GNAQ, HNF1A, IDH1, JAK2, KDR, KIT, KMT2A, KRAS, MAP2K1, MAP2K2, MSH3, MSH6, MTOR, NF1, NOTCH1, NRAS, NTRK1, PBRM1, PIK3CA, PIK3R1, PTCH1, PTEN, RAF1, RET, RNF43, SMAD4, SMO, TERT, and TP53. The set of candidate somatic variants was then filtered by a series of filters that were each designed to remove candidate somatic variants from the set of candidate somatic variants.

One such filter removed from the set of candidate somatic variants each respective candidate somatic variant in the set of candidate somatic variants that is present in a repository of known germline variants.

Another such filter removed from the set of candidate somatic variants each respective candidate somatic variant in the set of candidate somatic variants that maps to a variant interval in a plurality of variant intervals, where each respective variant interval in the plurality of variant intervals is identified as having a pre-test odds of a positive variant call that is less than a pre-test odds threshold value based upon a prevalence of a corresponding one or more training variants, which are each above a limit of detection and map to the respective variant interval, in a plurality of tumor-normal matched samples for colorectal cancer obtained from a first cohort of training subjects having colorectal cancer with the proviso that no variant detected in a second cohort of healthy samples maps to the respective variant interval.

Another such filter removed from the set of candidate somatic variants each respective candidate somatic variant in the set of candidate somatic variants that was identified as an artifactual variant.

Still another filter removed from the set of candidate somatic variants each respective candidate somatic variant in the set of candidate somatic variants that failed to be represented by at least one cell-free DNA fragment in the second plurality of cell-free DNA fragments in which both strands of the at least one cell-free DNA fragment were identified in one or more sequence reads of the second plurality of sequence reads.

Once the filtering was completed for each subject, a second call for molecular residual disease was made when there remained a candidate variant in the set of candidate variants after application of the filtering, or a second call against molecular residual disease when no candidate variant remains in the set of candidate variants after application of the procedure.

As illustrated in FIG. 3, each subject was designated as MRD positive when either their first or second call was for MRD. Each subject was designated as MRD negative when both their first and second call were against MRD.

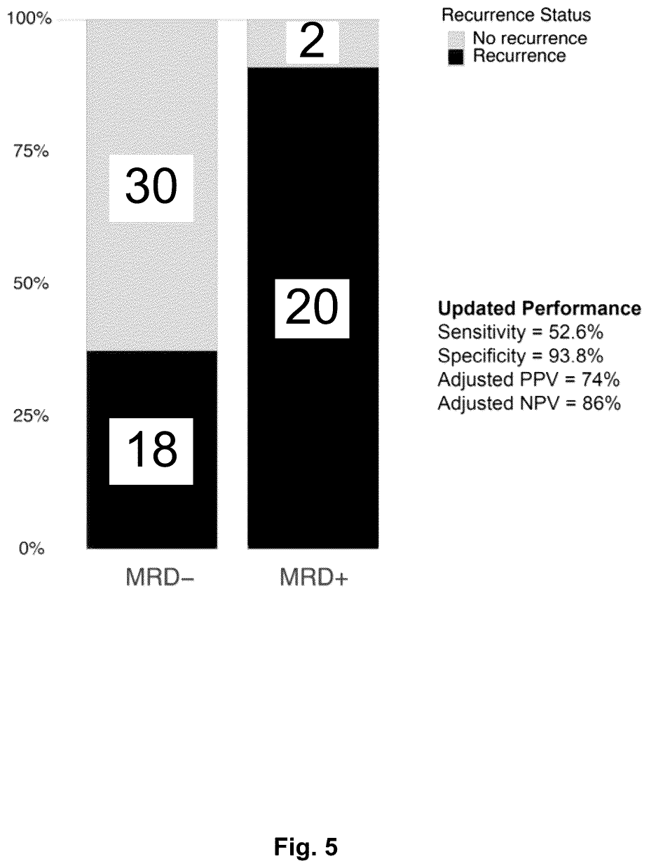

The MRD calls made at the landmark four week post-surgery time point by the model were compared to actual clinical outcome of each of the 70 subjects. The model performance is illustrated in FIG. 5. As illustrated in FIG. 5, of the 48 subjects that were clinically MRD negative, the model correctly predicted that 30 of the subjects were MRD negative and incorrectly predicted that 18 of the subjects were MRD positive. As illustrated in FIG. 5, of the 22 subjects that were clinically MRD positive, the model correctly predicted that 20 of the subjects were MRD positive and incorrectly predicted that 2 of the subjects were MRD negative. Accordingly, the model had a sensitivity of 52.6 percent, and a specificity of 93.9 percent.

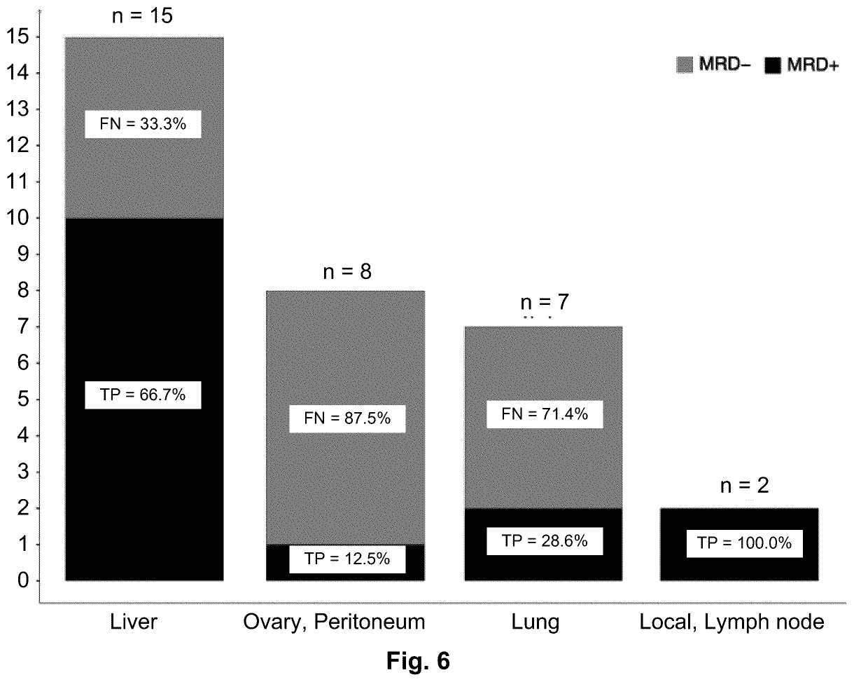

Recurrence of cancer occurred in various tissues. FIG. 6 illustrates model performance broken down by site of clinical recurrence. From FIG. 6, it is seen that liver site of recurrence had the highest true positive (TP) rate of 66.7% versus 20% for ovary, peritoneum and lung combined (p=0.011; Fisher's exact test). In FIG. 6, TP stands for true positive, and FN stands for false negative.

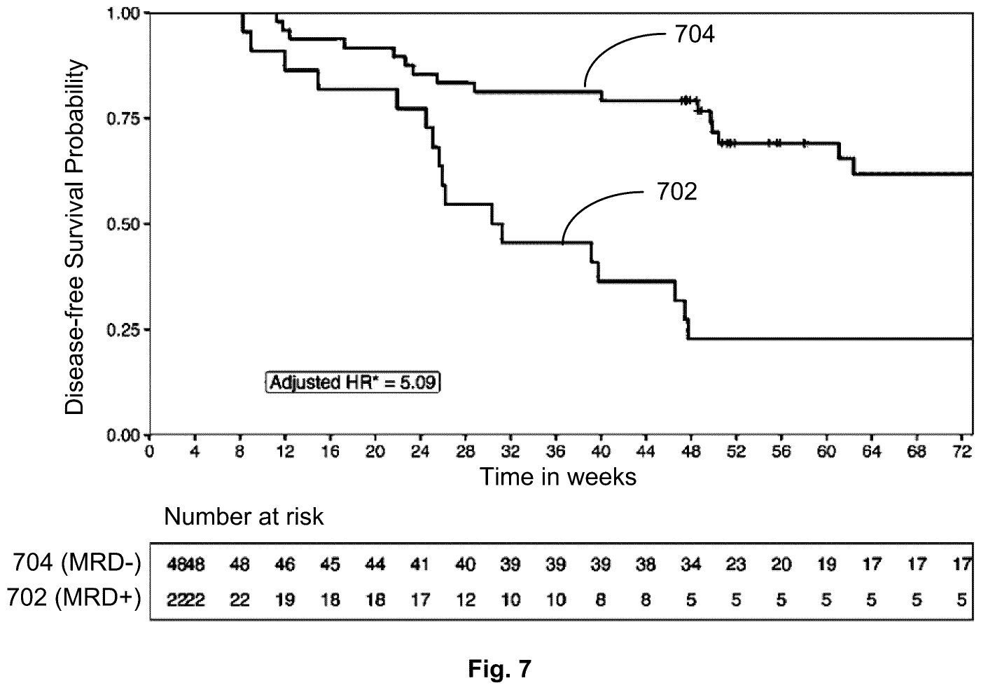

FIG. 7 illustrates disease free survival (DFS) by landmark 1-month post-surgery MRD status for subjects with greater than one year follow-up. The Kaplan-Meier (KM) estimates were obtained based on an enriched sample (50% recurrence rate) where the Adjusted hazard ratio (HR*) was the hazard ratio adjusted by the anticipated true recurrence rate of 24%. The adjusted median DFS time for MRD+ is 39.3 weeks and for MRD− is >72 weeks.

### Example 8: Model Performance in Accordance with the Embodiment of FIG. 4

In this example, the performance of a model in accordance with an embodiment drawn from FIG. 4 was tested. The model was run for each of 80 subjects having early stage colorectal cancer (stage 2 or 3) in order to determine whether each of the subjects had a positive or negative molecular residual disease status for the cancer condition. Each of the subjects underwent curative surgery.

Four weeks after the curative surgery, a blood sample was collected from each subject. A corresponding nucleic acid sequence of each cell-free DNA fragment in a first plurality of cell-free DNA fragments, from a first plurality of sequence reads of a first sequencing reaction was obtained for each subject using their blood sample. The first sequencing reaction was a methylation sequencing of the first plurality of cell-free DNA fragments from the blood sample of the subject. Each respective nucleic acid sequence in the first plurality of nucleic acid sequences comprised a methylation pattern for a corresponding cell-free DNA fragment in the first plurality of cell-free DNA fragments for each subject.

For each subject, each cell-free DNA that fails to satisfy a methylation rate threshold was removed from their plurality of cell-free DNA fragments in accordance with blocks 417-424 of FIG. 4.

For each subject, a corresponding number of unexpected circulating-tumor (ctDNA) fragments mapping to each respective region in a plurality of regions of a reference human genome was determined (e.g., using a methylation pattern of each nucleic acid sequence in the first plurality of nucleic acid sequences.), in accordance with block 426 of FIG. 4.

For each respective subject, an unexpected fragments per million value for the first liquid biopsy sample from the respective subject was determined using the corresponding number of unexpected ctDNA fragments in each respective region of the plurality of regions in accordance with block 428 of FIG. 4.

For each respective subject, the corresponding number of unexpected circulating-tumor DNA (ctDNA) fragments mapping to each region in the plurality of regions was used to determine an unlikely fragments per million value across the plurality of regions for the respective subject in accordance with block 429 of FIG. 4.

For each respective subject, a first threshold was applied to the unlikely fragments per million value to provide: a first call for molecular residual disease when the unlikely fragments per million value satisfied the first threshold or a first call against molecular residual disease when the unlikely fragments per million value failed to satisfy the first threshold in accordance with block 430 of FIG. 4.

In addition to the first call, a second call was made for each of the subjects. To make the second call, there was obtained, from a second sequencing reaction, for each of the subjects, a corresponding sequence of each cell-free DNA fragment in a second plurality of cell-free DNA fragments from the above-described blood sample of the subject, thereby obtaining a second plurality of sequence reads.

For each of the subjects, the second plurality of sequence reads was used to identify each candidate somatic variant in a set of candidate somatic variants, where each candidate somatic variant in the set of candidate somatic variants was a single nucleic acid variant (SNV). In various embodiments, the set of candidate somatic variants may not all be SNVs. For example, in some embodiments the set of candidate somatic variants may include deletion mutations or insertion mutations. In various embodiments, the set of candidate somatic variants may be tailored to a cancer type associated with the subject's diagnosis. In some embodiments, this set of candidate somatic variants included any variants arising in the following genes: AKT1, ALK, APC, AR, ARID1A, ATM, B2M, BRAF, BRCA2, CCND1, CDKN2A, CTNNB1, ERBB2, FBXW7, FGFR2, FGFR3, FGFR4, GNA11, GNAQ, HNF1A, IDH1, JAK2, KDR, KIT, KMT2A, KRAS, MAP2K1, MAP2K2, MSH3, MSH6, MTOR, NF1, NOTCH1, NRAS, NTRK1, PBRM1, PIK3CA, PIK3R1, PTCH1, PTEN, RAF1, RET, RNF43, SMAD4, SMO, TERT, and TP53. The set of candidate somatic variants was then filtered by a series of filters that were each designed to remove candidate somatic variants from the set of candidate somatic variants.

One such filter removed from the set of candidate somatic variants each respective candidate somatic variant in the set of candidate somatic variants that is present in a repository of known germline variants.

Another such filter removed from the set of candidate somatic variants each respective candidate somatic variant in the set of candidate somatic variants that maps to a variant interval in a plurality of variant intervals, where each respective variant interval in the plurality of variant intervals is identified as having a pre-test odds of a positive variant call that is less than a pre-test odds threshold value based upon a prevalence of a corresponding one or more training variants, which are each above a limit of detection and map to the respective variant interval, in a plurality of tumor-normal matched samples for colorectal cancer obtained from a first cohort of training subjects having colorectal cancer with the proviso that no variant detected in a second cohort of healthy samples maps to the respective variant interval.

Another such filter removed from the set of candidate somatic variants each respective candidate somatic variant in the set of candidate somatic variants that was identified as an artifactual variant.

Still another filter removed from the set of candidate somatic variants each respective candidate somatic variant in the set of candidate somatic variants that failed to be represented by at least one cell-free DNA fragment in the second plurality of cell-free DNA fragments in which both strands of the at least one cell-free DNA fragment were identified in one or more sequence reads of the second plurality of sequence reads.

Once the filtering was completed for each subject, a second call for molecular residual disease was made when there remained a candidate variant in the set of candidate variants after application of the filtering, or a second call against molecular residual disease when no candidate variant remains in the set of candidate variants after application of the procedure.

As illustrated in FIG. 3, each subject was designated as MRD positive when either their first or second call was for MRD. Each subject was designated as MRD negative when both their first and second call were against MRD.

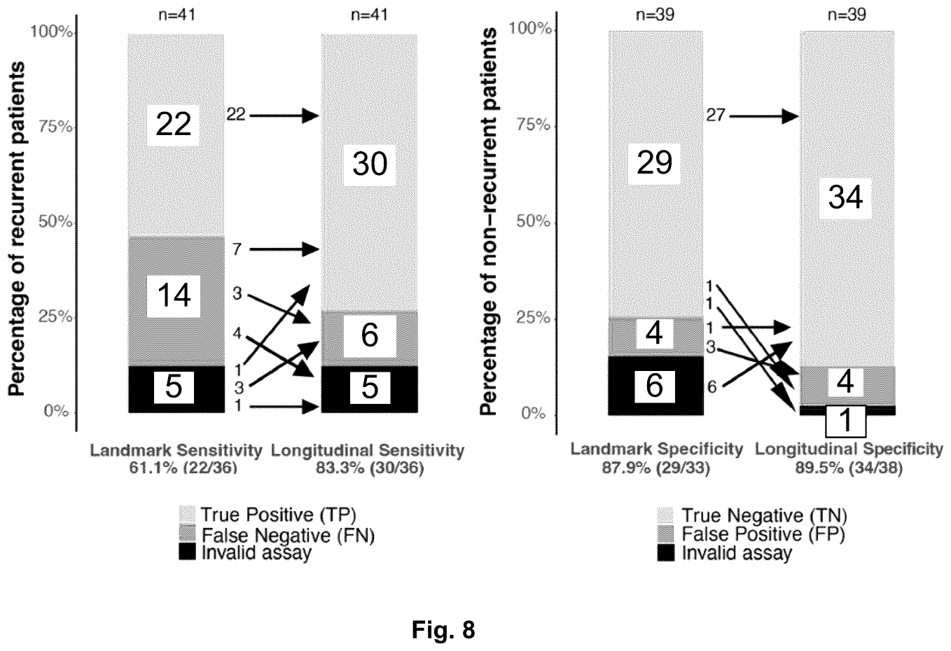

The MRD calls made at the landmark four week post-surgery time point by the model were compared to actual clinical outcome of each of the 70 subjects. The model performance is illustrated in FIG. 8.

As illustrated in FIG. 8, left panel, of the 41 subjects that were clinically MRD positive, the model correctly predicted that 20 of the subjects were MRD positive and incorrectly predicted that 14 of the subjects were MRD negative, while 5 of the subjects had invalid assays. Accordingly, the model had a sensitivity at landmark of 51.1 percent.

As illustrated in FIG. 8, right panel, of the 39 subjects that were clinically MRD negative, the model correctly predicted that 29 of the subjects were MRD negative and incorrectly predicted that 4 of the subjects were MRD positive, while the assays for 6 of the clinically MRD negative subjects were invalidated. Accordingly, the model had a specificity at landmark of 87.9 percent.

In addition to landmark assays, longitudinal assays in accordance with FIG. 4 were performed every three months after death. FIG. 8 further illustrates model performance for these longitudinal model predictions.

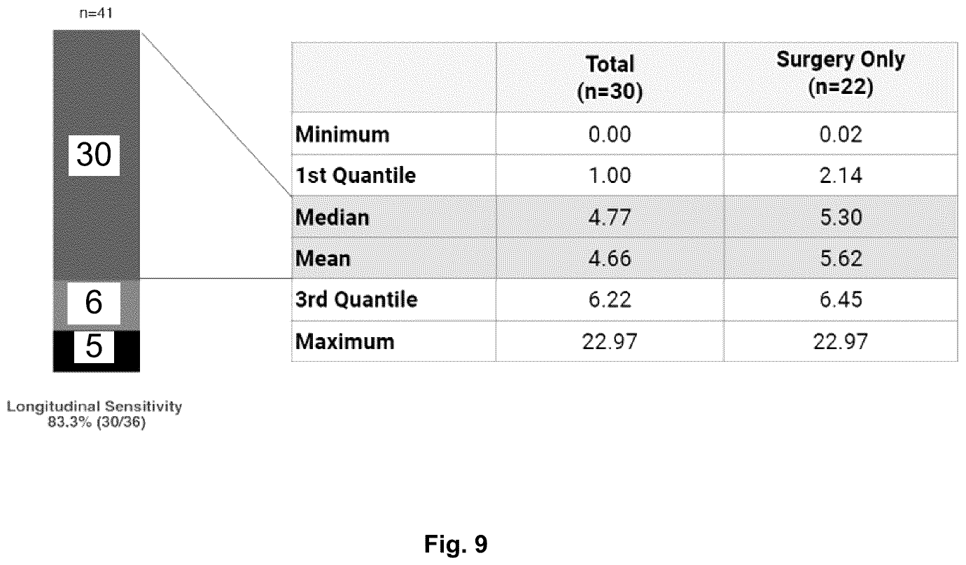

FIG. 9 illustrates distribution of lead time (time from first MRD+ call to date of recurrence or death) for TP (n=30) subjects. Overall mean lead time defined from first MRD+ to recurrence is 4.66 months. For subjects with surgery only treatment, the mean lead time was 5.62 months.

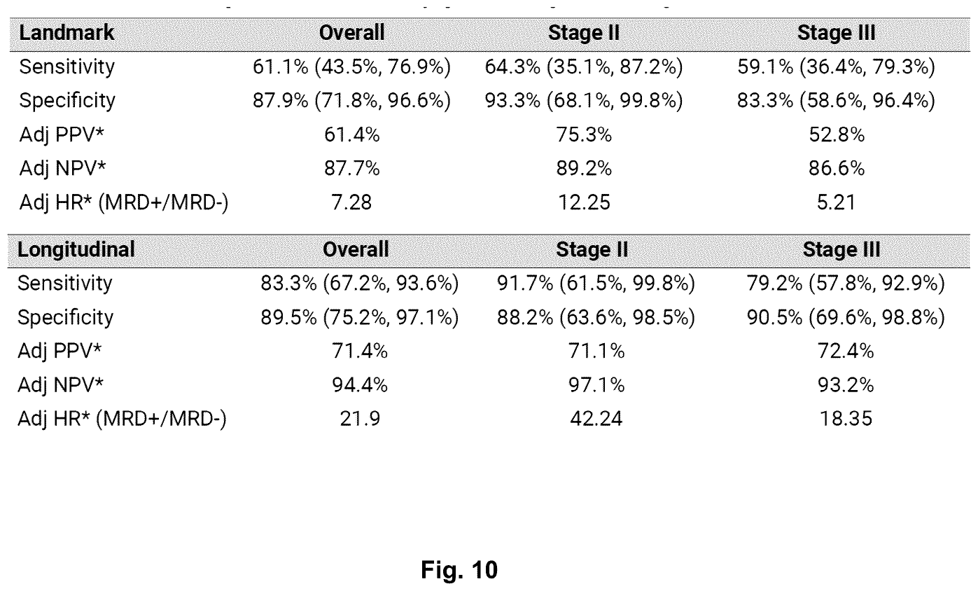

FIG. 10 illustrates clinical landmark performance (top) and clinical longitudinal performance (bottom). Adj PPV*, Adj NPV*, and Adj HR* were the estimates based on the anticipated true recurrence rate of 24%.

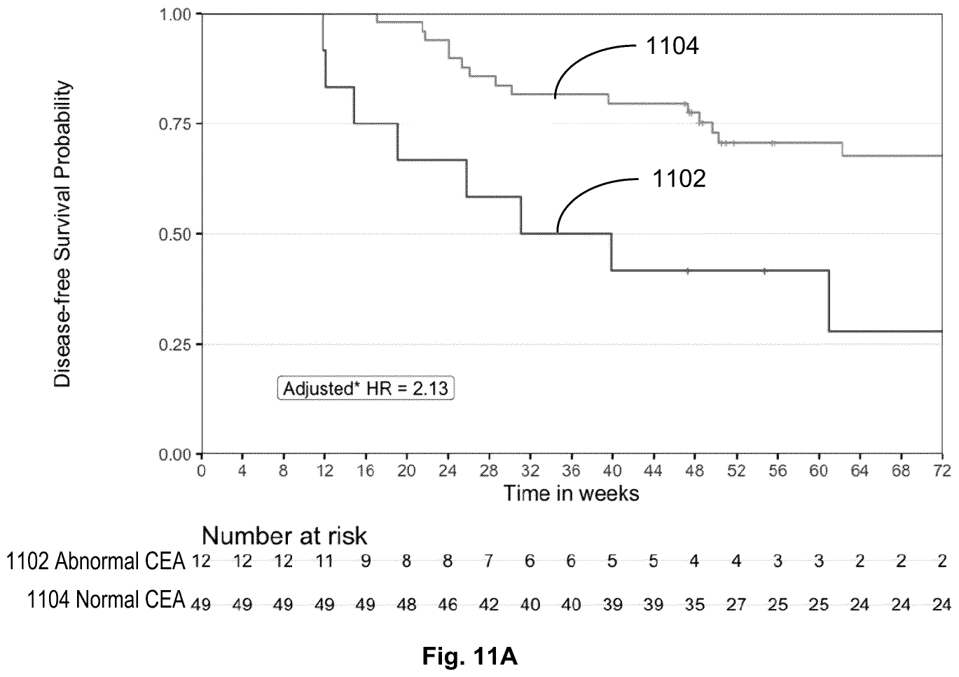

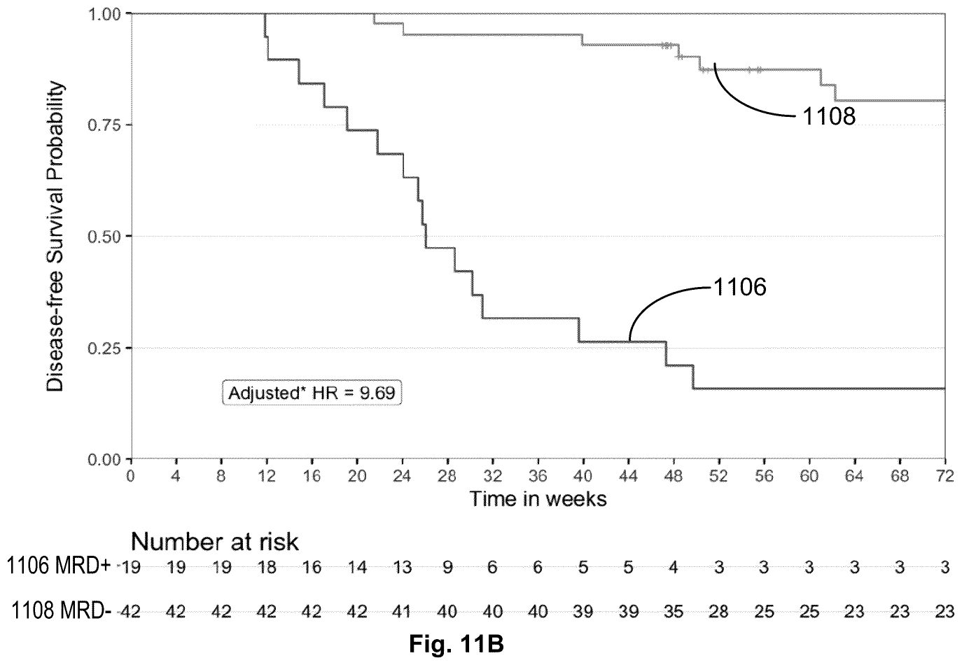

FIGS. 11A and 11B illustrate adjusted hazard ratio (HR) for the disclosed model was nearly 5-fold higher compared to carcinoembryonic antigen (CEA) testing at 12 weeks post surgery. Adjusted HR* was the hazard ratio adjusted by anticipated true recurrence rate (24%). The adjusted median disease free survival (DFS) time for MRD+ is 25.1 weeks (6.3 months) versus not reached within 72 weeks (18 months) for MRD−.

### Additional Embodiments

Another aspect of the present disclosure provides a computer system comprising one or more processors and a non-transitory computer-readable medium including computer-executable instructions that, when executed by the one or more processors, cause the processors to perform any of the methods and/or embodiments disclosed herein.

Yet another aspect of the present disclosure provides a non-transitory computer-readable storage medium having stored thereon program code instructions that, when executed by a processor, cause the processor to perform any of the methods and/or embodiments disclosed herein.

Although inventions have been particularly shown and described with reference to a preferred embodiment and various alternate embodiments, it will be understood by persons skilled in the relevant art that various changes in form and details can be made therein without departing from the spirit and scope of the invention.

### EQUIVALENTS AND INCORPORATION BY REFERENCE

All references cited herein are incorporated by reference to the same extent as if each individual publication, database entry (e.g., Genbank sequences or GeneID entries), patent application, or patent, was specifically and individually indicated to be incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

### ADDITIONAL CONSIDERATIONS

The foregoing description of the embodiments has been presented for the purpose of illustration; it is not intended to be exhaustive or to limit the patent rights to the precise forms disclosed. Persons skilled in the relevant art will appreciate that many modifications and variations are possible in light of the above disclosure.

Any feature mentioned in one claim category, e.g., method, can be claimed in another claim category, e.g., computer program product, system, storage medium, as well. The dependencies or references back in the attached claims are chosen for formal reasons only. However, any subject matter resulting from a deliberate reference back to any previous claims (in particular multiple dependencies) can be claimed as well, so that any combination of claims and the features thereof is disclosed and can be claimed regardless of the dependencies chosen in the attached claims. The subject matter, in some embodiments, includes not only the combinations of features as set out in the disclosed embodiments but also any other combination of features from different embodiments. Various features mentioned in the different embodiments can be combined with explicit mentioning of such combination or arrangement in an example embodiment or without any explicit mentioning. Furthermore, any of the embodiments and features described or depicted herein, in some embodiments, are claimed in a separate claim and/or in any combination with any embodiment or feature described or depicted herein or with any of the features.

Some portions of this description describe the embodiments in terms of algorithms and symbolic representations of operations on information. These operations and algorithmic descriptions, while described functionally, computationally, or logically, are understood to be implemented by computer programs or equivalent electrical circuits, microcode, or the like. Furthermore, it has also proven convenient at times, to refer to these arrangements of operations as engines, without loss of generality. The described operations and their associated engines are, in some embodiments, embodied in software, firmware, hardware, or any combinations thereof.

Any of the steps, operations, or processes described herein, in some embodiments, are performed or implemented with one or more hardware or software engines, alone or in combination with other devices. In one embodiment, a software engine is implemented with a computer program product comprising a computer-readable medium containing computer program code, which can be executed by a computer processor for performing any or all of the steps, operations, or processes described. The term “steps” does not mandate or imply a particular order. For example, while this disclosure describes, in some embodiments, a process that includes multiple steps sequentially with arrows present in a flowchart, the steps in the process do not need to be performed by the specific order claimed or described in the disclosure. In some implementations, some steps are performed before others even though the other steps are claimed or described first in this disclosure. Likewise, any use of (i), (ii), (iii), etc., or (a), (b), (c), etc. in the specification or in the claims, unless specified, is used to better enumerate items or steps and also does not mandate a particular order.

1. A method of determining whether a subject has a positive or negative molecular residual disease status for a cancer condition, the method comprising:
at a computer system having one or more processors, and memory storing one or more programs for execution by the one or more processors:
A) obtaining a corresponding nucleic acid sequence of each cell-free DNA fragment in a first plurality of cell-free DNA fragments, from a first plurality of sequence reads of a first sequencing reaction, wherein the first sequencing reaction is a methylation sequencing of the first plurality of cell-free DNA fragments from a first liquid biopsy sample of the subject, and wherein each respective nucleic acid sequence in the first plurality of nucleic acid sequences comprises a methylation pattern for a corresponding cell-free DNA fragment in the first plurality of cell-free DNA fragments;
B) determining a corresponding number of circulating-tumor DNA (ctDNA) fragments mapping to each respective region in a plurality of regions of one or more first reference sequences of the species of the subject using a methylation pattern of each nucleic acid sequence in the first plurality of nucleic acid sequences;
C) determining a corresponding expected number of noise fragments in each respective region of the plurality of regions of the one or more first reference sequences of the species of the subject based on a corresponding distribution using an observed sequencing depth from the first sequence reaction and a learned background emission rate for the respective region;
D) determining an excess fragments per million value for the first liquid biopsy sample from the corresponding number of ctDNA fragments in each respective region of the plurality of regions of the one or more first reference sequences observed in the first liquid biopsy sample in excess of the corresponding expected number of noise fragments in the respective region;
E) correcting the excess fragments per million value for the first liquid biopsy sample by an observed CHG methylation level to obtain a corrected excess fragments per million value;
F) applying a first threshold to the corrected excess fragments per million value to provide:
a first call for molecular residual disease when the corrected excess fragments per million value satisfies the first threshold or
a first call against molecular residual disease when the corrected excess fragments per million value fails to satisfy the first threshold; and
G) providing:
an indication that the subject has positive molecular residual disease status for the cancer condition when the applying F) provides the first call for molecular residual disease, or
an indication that the subject has negative molecular residual disease status for the cancer condition when the applying F) provides the first call against molecular residual disease.

2. The method of claim 1, further comprising:
H) obtaining, from a second sequencing reaction, a corresponding sequence of each cell-free DNA fragment in a second plurality of cell-free DNA fragments in a second liquid biopsy sample of the subject, thereby obtaining a second plurality of sequence reads;
I) using the second plurality of sequence reads to identify each candidate somatic variant in a set of candidate somatic variants, wherein each candidate somatic variant in the set of candidate somatic variants is a single nucleotide variant (SNV);
J) filtering the set of candidate somatic variants by a procedure comprising:
i) removing from the set of candidate somatic variants each respective candidate somatic variant in the set of candidate somatic variants that is present in a repository of known germline variants,
ii) removing from the set of candidate somatic variants each respective candidate somatic variant in the set of candidate somatic variants that maps to a variant interval in a plurality of variant intervals, wherein each respective variant interval in the plurality of variant intervals is identified as having a pre-test odds of a positive variant call that is less than a pre-test odds threshold value based upon a prevalence of a corresponding one or more training variants, which are each above a limit of detection and map to the respective variant interval, in a plurality of tumor-normal matched samples for the cancer condition obtained from a first cohort of training subjects having the cancer condition with the proviso that no variant detected in a second cohort of healthy samples maps to the respective variant interval;
iii) removing from the set of candidate somatic variants each respective candidate somatic variant in the set of candidate somatic variants that is identified as an artifactual variant, and
iv) removing from the set of candidate somatic variants each respective candidate somatic variant in the set of candidate somatic variants that fails to be represented by at least one cell-free DNA fragment in the second plurality of cell-free DNA fragments in which both strands of the at least one cell-free DNA fragment are identified in one or more sequence reads of the second plurality of sequence reads, and
K) providing:
a second call for molecular residual disease when there remains a candidate variant in the set of candidate variants after application of the procedure, or
a second call against molecular residual disease when no candidate variant remains in the set of candidate variants after application of the procedure; and
wherein, the providing G) provides the indication that the subject has positive molecular residual disease status for the cancer condition when the applying F) provides the first call for molecular residual disease or the providing K) provides the second call for molecular residual disease.

3. (canceled)

4. The method of claim 2, wherein the procedure further comprises:
v) removing from the set of candidate variants each respective candidate somatic variant in the set of candidate somatic variants that (a) maps to a repeat region in the one or more second reference sequences of the species and (b) is not annotated as a known somatic mutation in a database of known somatic mutations for the species of the subject.

5. The method of claim 2, wherein the providing G) provides the indication that the subject has negative molecular residual disease status for the cancer condition when the applying F) provides the first call against molecular residual disease and the providing K) provides the second call against molecular residual disease.

6. The method of claim 2, wherein the first liquid biopsy sample and the second liquid biopsy sample are the same liquid biopsy sample.

7. The method of claim 2, wherein the first liquid biopsy sample and the second liquid biopsy sample are different liquid biopsy samples.

8. (canceled)

9. The method of claim 1, wherein the first liquid biopsy sample comprises blood, whole blood, peripheral blood, plasma, serum, or lymph of the subject.

10. (canceled)

11. The method of claim 2, wherein the second liquid biopsy sample comprises blood, whole blood, peripheral blood, plasma, serum, or lymph of the subject.

12. The method of claim 1, wherein a volume of the first liquid biopsy sample is less than 30 mL.

13. The method of claim 1, wherein the second sequencing reaction is a panel-based sequencing reaction of a plurality of loci.

14. The method of claim 13, wherein the plurality of loci is sequenced at an average sequence depth of at least 250× by the second sequencing reaction.

15. (canceled)

16. The method of claim 13, wherein
the one or more first reference sequences is a human reference genome,
the one or more second reference sequences is the human reference genome,
the plurality of regions comprises 1000 or more regions cumulatively mapping to between four megabases and ten megabases of the human reference genome, and
the plurality of loci comprises 50 or more loci cumulatively mapping to between 0.1 megabase and 1 megabase of the human reference genome.

17. The method of claim 1, wherein the first sequencing reaction is a whole genome methylation sequencing.

18. The method of claim 1, wherein
the first plurality of sequence reads comprises at least 50,000 sequence reads, and
the second plurality of sequence reads comprises at least 50,000 sequence reads.

19. (canceled)

20. The method of claim 1, wherein the cancer condition is a particular type of cancer or a particular stage of a particular type of cancer.

21. (canceled)

22. The method of claim 2, the method further comprising generating a report for the subject comprising the identity of candidate variants remaining in the set of candidate variants after running the procedure.

23. The method of claim 22, wherein the report further comprises a therapeutic match for the subject based on an identity of one or more of the candidate variants remaining in the set of candidate variants after running the procedure.

24. The method of claim 2, wherein the using I) further identifies each respective candidate somatic variant in the set of candidate somatic variants by applying a variant caller to the second plurality of sequence reads with a restriction that the variant caller determines that each respective candidate somatic variant in the set of candidate somatic variants has a variant allele frequency of at least 0.1 in the second plurality of sequence reads and that at least one cell-free DNA fragment in the second plurality of cell-free DNA fragments exhibits the respective candidate somatic variant.

25. The method of claim 2, wherein the procedure further comprises:
v) removing from the set of candidate somatic variants each respective candidate somatic variant in the set of candidate somatic variants that maps to a region of clonal hematopoiesis of indeterminate potential (CHIP).

26. The method of claim 25, wherein the region of CHIP is ASXL1, BCOR, BCORL1, CBL, CREBBP, CUX1, DNMT3A, GNB1, JAK2, PPM1D, PRPF8, SETDB1, SF3B1, SRSF2, TET2, U2AF1, or any combination thereof.

27. The method of claim 25, wherein the region of CHIP is TET2, DNMT3A, ASXL1, SF3B1, or any combination thereof.

28. The method of claim 25, wherein the region of CHIP is TET2, DNMT3A, ASXL1, SF3B1, CBL, U2AF1, IDH2,2,3, MYD88,13, EP300, CDKN2C, HNF1A, or any combination thereof.

29. The method of claim 2, wherein the procedure comprises removing from the set of candidate allele variants each candidate variant in the set of candidate allele variants that has a variant allele fraction exceeding an upper threshold fraction in the second plurality of sequence reads.

30. The method of claim 2, wherein the procedure comprises removing from the set of candidate allele variants each candidate variant in the set of candidate allele variants that is observed in a cohort of healthy subjects.

31. The method of claim 2, wherein the procedure comprises removing from the set of candidate allele variants each respective candidate variant in the set of candidate allele variants in which the second sequencing reaction produced a coverage depth of less than a threshold amount for the respective locus in one or more second reference sequences of the species of the subject that the candidate somatic variant maps to.

32. The method of claim 1, wherein the method further comprises removing, from the first plurality of cell-free DNA fragments, each cell-free DNA fragment that fails to satisfy a methylation rate threshold prior to the determining B) or the determining C).

33. (canceled)

34. The method of claim 1, wherein the subject is taking an adjuvant therapy for the cancer condition and the method further comprises:
determining that the subject has positive molecular residual disease status for the cancer condition in accordance with the providing G); and
adjusting the adjuvant therapy, wherein the adjuvant therapy is chemotherapy, radiation therapy, hormone therapy, or immunotherapy.

35. (canceled)

36. The method of claim 34, wherein the adjusting the adjuvant therapy comprises increasing a dosage of the adjuvant therapy, decreasing a dosage of the adjuvant therapy, or ceasing the adjuvant therapy.

37. A computer system comprising:
one or more processors; and
a non-transitory computer-readable medium including computer-executable instructions that, when executed by the one or more processors, cause the processors to perform a method comprising:
A) obtaining a corresponding nucleic acid sequence of each cell-free DNA fragment in a first plurality of cell-free DNA fragments, from a first plurality of sequence reads of a first sequencing reaction, wherein the first sequencing reaction is a methylation sequencing of the first plurality of cell-free DNA fragments from a first liquid biopsy sample of the subject, and wherein each respective nucleic acid sequence in the first plurality of nucleic acid sequences comprises a methylation pattern for a corresponding cell-free DNA fragment in the first plurality of cell-free DNA fragments:
B) determining a corresponding number of circulating-tumor DNA (ctDNA) fragments mapping to each respective region in a plurality of regions of one or more first reference sequences of the species of the subject using a methylation pattern of each nucleic acid sequence in the first plurality of nucleic acid sequences;
C) determining a corresponding expected number of noise fragments in each respective region of the plurality of regions of the one or more first reference sequences of the species of the subject based on a corresponding distribution using an observed sequencing depth from the first sequence reaction and a learned background emission rate for the respective region;
D) determining an excess fragments per million value for the first liquid biopsy sample from the corresponding number of ctDNA fragments in each respective region of the plurality of regions of the one or more first reference sequences observed in the first liquid biopsy sample in excess of the corresponding expected number of noise fragments in the respective region;
E) correcting the excess fragments per million value for the first liquid biopsy sample by an observed CHG methylation level to obtain a corrected excess fragments per million value;
F) applying a first threshold to the corrected excess fragments per million value to provide:
a first call for molecular residual disease when the corrected excess fragments per million value satisfies the first threshold or
a first call against molecular residual disease when the corrected excess fragments per million value fails to satisfy the first threshold; and
G) providing:
an indication that the subject has positive molecular residual disease status for the cancer condition when the applying F) provides the first call for molecular residual disease, or
an indication that the subject has negative molecular residual disease status for the cancer condition when the applying F) provides the first call against molecular residual disease.

38. A non-transitory computer-readable storage medium having stored thereon program code instructions that, when executed by a processor, cause the processor to perform a method comprising:
A) obtaining a corresponding nucleic acid sequence of each cell-free DNA fragment in a first plurality of cell-free DNA fragments, from a first plurality of sequence reads of a first sequencing reaction, wherein the first sequencing reaction is a methylation sequencing of the first plurality of cell-free DNA fragments from a first liquid biopsy sample of the subject, and wherein each respective nucleic acid sequence in the first plurality of nucleic acid sequences comprises a methylation pattern for a corresponding cell-free DNA fragment in the first plurality of cell-free DNA fragments;
B) determining a corresponding number of circulating-tumor DNA (ctDNA) fragments mapping to each respective region in a plurality of regions of one or more first reference sequences of the species of the subject using a methylation pattern of each nucleic acid sequence in the first plurality of nucleic acid sequences;
C) determining a corresponding expected number of noise fragments in each respective region of the plurality of regions of the one or more first reference sequences of the species of the subject based on a corresponding distribution using an observed sequencing depth from the first sequence reaction and a learned background emission rate for the respective region:
D) determining an excess fragments per million value for the first liquid biopsy sample from the corresponding number of ctDNA fragments in each respective region of the plurality of regions of the one or more first reference sequences observed in the first liquid biopsy sample in excess of the corresponding expected number of noise fragments in the respective region;
E) correcting the excess fragments per million value for the first liquid biopsy sample by an observed CHG methylation level to obtain a corrected excess fragments per million value;
F) applying a first threshold to the corrected excess fragments per million value to provide:
a first call for molecular residual disease when the corrected excess fragments per million value satisfies the first threshold or
a first call against molecular residual disease when the corrected excess fragments per million value fails to satisfy the first threshold; and
G) providing:
an indication that the subject has positive molecular residual disease status for the cancer condition when the applying F) provides the first call for molecular residual disease, or
an indication that the subject has negative molecular residual disease status for the cancer condition when the applying F) provides the first call against molecular residual disease.

39.-78. (canceled)
